(19) United States
(12) Patent Application Publication
(10) Pub. No.: US 2024/0116945 A1
(43) Pub. Date: Apr. 11, 2024

(54) EXATECAN-DERIVED TOPOISOMERASE-1 INHIBITORS PHARMACEUTICAL COMPOSITIONS, AND USES THEREOF

(71) Applicant: Merck Sharp & Dohme LLC, Rahway, NJ (US)

(72) Inventors: Vlad Bacauanu, San Francisco, CA (US); Manoj B. Charati, Redwood City, CA (US); Rebecca Elizabeth Johnson, Oakland, CA (US); Simon B. Lang, San Francisco, CA (US); Ryan Quiroz, San Francisco, CA (US); W. Michael Seganish, Castro Valley, CA (US); Song Yang, Springfield, NJ (US); Nancy S. Zepeda, San Francisco, CA (US)

(73) Assignee: Merck Sharp & Dohme LLC, Rahway, NJ (US)

(21) Appl. No.: 18/240,392

(22) Filed: Aug. 31, 2023

Related U.S. Application Data
(60) Provisional application No. 63/403,515, filed on Sep. 2, 2022.
(60) Provisional application No. 63/421,844, filed on Nov. 2, 2022.
(60) Provisional application No. 63/488,007, filed on Mar. 2, 2023.

(51) Int. Cl. C07D 491/22; A61P 35/00

(52) U.S. Cl. CPC C07D 491/22; A61P 35/00

(57) ABSTRACT

The present disclosure is directed to alcohol and amine containing exatecan derivatives, and pharmaceutically acceptable salts or solvates thereof, comprising a structure of formula I:

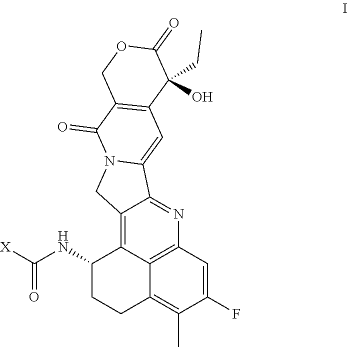

wherein X is defined in the disclosure. The disclosure is also directed to pharmaceutical compositions comprising these compounds and the use of these compounds and compositions in the prevention or treatment of cancers and/or tumors.

### CROSS-REFERENCE TO RELATED APPLICATIONS

This application claims the benefit of priority to U.S. Provisional Application No. 63/403,515, filed Sep. 2, 2022, U.S. Provisional Application No. 63/421,844, filed Nov. 2, 2022, and U.S. Provisional Application No. 63/488,007, filed Mar. 2, 2023, the disclosure of which is incorporated herein by its entirety.

### BACKGROUND OF THE INVENTION

The present disclosure relates to topoisomerase-1 inhibitors derived from the exatecan scaffold, which can be used for oncologic therapies. The topoisomerase-1 inhibitors are cytotoxic chemotherapeutic derivatives with a camptothecin core. Documents reporting the use of the camptothecin derivative, exatecan (chemical name: (1S, 9S)-1-amino-9-ethyl-5-fluoro-2,3-dihydro-9-hydroxy-4-methyl-1H,12H-benzo[de]pyrano[3′,4,6,7]imidazo[1,2-b] quinoline-10,13(9H,15H)-dione) are disclosed in WO2014057687, U.S. Ser. No. 11/103,593, U.S. Pat. Nos. 9,808,537, 7,091,186, US 2010/0062008, WO2015057699, Clinical Cancer Research (2016) 22 (20): 5097-5108, and Cancer Sci (2016) 107: 1039-1046. See also WO2022068878, WO2017062271, CN113816969, CN112125915, and US20210353764.

### SUMMARY OF THE INVENTION

The compounds of this disclosure represent a class of topoisomerase-1 inhibitors derived from the exatecan scaffold. Specifically, the compounds are described as alcohol and amine containing exatecan amides, and analogs therein. The compounds are cytotoxic and can be applied as chemotherapeutic drugs in oncologic settings, for example as an anti-tumor agent. The compounds are potent, novel in structure, active across multiple cancer cell lines.

For each of the following embodiments, any variable not explicitly defined in the embodiment is as defined in Formula (I). In each of the embodiments described herein, each variable is selected independently of the other unless otherwise noted.

In one embodiment, the present disclosure provides alcohol and amine containing exatecan derivatives, and pharmaceutically acceptable salts, solvates, or steroisomer thereof, comprising a structure of formula I:

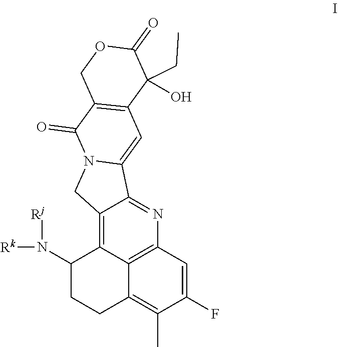

wherein:
R^{k} is selected from hydrogen, —C₁₋₆ alkyl, (CH₂)ₙC(O)NHC₁₋₆ alkyl, (CH₂)ₙC₆₋₁₀ aryl and

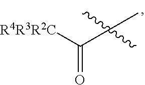

said alkyl and aryl optionally substituted with 1 to 3 groups of hydroxyl, —C₁₋₆ alkylOH, and said alkyl optionally substituted with 1 to 10 groups of halogen;

R^{j} represents hydrogen or C₁₋₆ alkyl, said alkyl optionally substituted with 1 to 10 halogen;

R² and R³ are independently selected from hydrogen, —C₁₋₆ alkyl, OH, —C₁₋₆ alkylOH, halogen, —C₁₋₉haloalkyl, —(CH₂)ₙNH₂, —NHC₁₋₆alkyl, —N(C₁₋₆ alkyl)₂, —C₃₋₆ cycloalkyl, —(CH₂)ₙC₆₋₁₀aryl, and —(CH₂)ₙOC₁₋₆alkyl, that R² and R³ are not both halogen at the same time;

R⁴ is selected from C₁₋₆ alkyl, OH, —C₁₋₆ alkylOH, —CH(OH)C₁₋₆ alkyl, —C₁₋₉haloalkyl, halogen, —C₃₋₆cycloalkyl, —(CH₂)ₙOC₁₋₃alkyl, —(CH₂)ₙOC₁₋₉haloalkyl, —CR^{x}R^{y}C₁₋₆ alkylOH, —(CH₂)ₙNH₂, —NHC₁₋₆ alkyl, —N(C₁₋₆ alkyl)₂, and —NHC(O)C₁₋₆alkylNH₂;

R^{x} and R^{y} represent C₁₋₃ alkylene that combine to form a C₃₋₆cycloalkyl, or spirocycloalkyl;

each n independently represents 0, 1, 2, or 3.

An embodiment of the disclosure is realized when R^{j} is hydrogen.

Another embodiment of the disclosure is realized when R^{j} is C₁₋₆ alkyl, said alkyl optionally substituted with 1 to 5 groups of halogen. A subembodiment of this aspect of the invention is realized when R^{j} is C₁₋₆ alkyl substituted with 1 to 4 halogen.

An embodiment of this disclosure is realized when R^{k} is hydrogen.

Another embodiment of this disclosure is realized when R^{k} is —C₁₋₆ alkyl, said alkyl optionally substituted to 1 to 10 halogen, or 1 to 3 groups selected from hydroxyl and —C₁₋₆ alkylOH.

Another embodiment of this disclosure is realized when R^{k} is (CH₂)ₙC(O)NHC₁₋₆ alkyl, said alkyl optionally substituted to 1 to 10 halogen, or 1 to 3 groups selected from hydroxyl, and —C₁₋₆ alkylOH.

Another embodiment of this disclosure is realized when R^{k} is (CH₂)ₙC₆₋₁₀ aryl, said aryl optionally substituted with 1 to 3 groups selected from hydroxyl, and —C₁₋₆ alkylOH.

Another embodiment of this disclosure is realized when R^{k} is

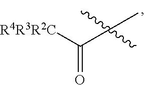

wherein R², R³ and R⁴ are as described herein.

An embodiment of this invention is realized by Formula I′, or a pharmaceutically acceptable salt, solvate, or stereoisomer thereof represented by structural formula:

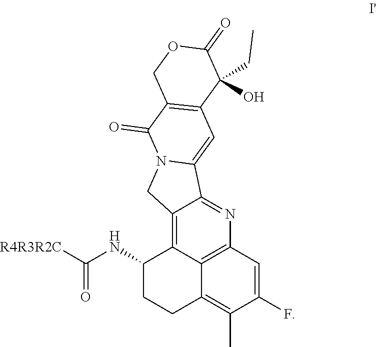

An embodiment of this disclosure is realized when no hydrogen atom in an alkyl containing substituent of R², R³ and R⁴ is deuterated.

Another embodiment of this disclosure is realized when at least one hydrogen atom in an alkyl containing substituent of R², R³ and R⁴ is deuterated.

Another embodiment of this disclosure is realized when n is 0. Another embodiment of this disclosure is realized when n is 1. Another embodiment of this disclosure is realized when n is 2. Still another embodiment of this disclosure is realized when n is 3.

Another embodiment of this disclosure is realized when R^{x} and R^{y} combine with the atoms to which they are attached to form cyclopropyl, cyclobutyl, cyclopentyl or cyclohexyl group.

Another embodiment of this disclosure is realized when R² is selected from hydrogen, C₁₋₆ alkyl, CH₂OC₁₋₆ alkyl, —(CH₂)ₙOH, —CH₂F, —CHF₂, —CF₃, —(CH₂)phenyl, cyclopropyl, cyclobutyl, cyclopentyl, cyclohexyl, —(CH₂)ₙNH₂, —NHCH₃, and —N(CH₃)₂. A subembodiment of this aspect of the disclosure is realized when R² is selected from hydrogen, C₁₋₆ alkyl, CH₂OCH₃, —OH, —CH₂OH, —CH₂F, —CHF₂, —CF₃, and —NH₂. Another subembodiment of this aspect of the disclosure is realized when R² is hydrogen. Another subembodiment of this aspect of the disclosure is realized when R² is C₁₋₆ alkyl selected from —CH₃, —CH₂CH₃, and —CH₂CH(CH₃)₂. Another subembodiment of this aspect of the disclosure is realized when R² is (CH₂)ₙOCH₃. Another subembodiment of this aspect of the disclosure is realized when R² is —OH or —CH₂OH. Another subembodiment of this aspect of the disclosure is realized when R² is —CH₂F, —CHF₂, or —CF₃. Still another subembodiment of this aspect of the disclosure is realized when R² is NH₂. Another subembodiment of this aspect of the disclosure is realized when any hydrogen atom in an alkyl containing substituent of R² is not deuterated.

Another embodiment of this disclosure is realized when R³ is selected from hydrogen, C₁₋₆ alkyl, CH₂OC₁₋₆ alkyl, —(CH₂)ₙOH, —CH₂F, —CHF₂, —CF₃, —(CH₂)phenyl, cyclopropyl, cyclobutyl, cyclopentyl, cyclohexyl, —(CH₂)ₙNH₂, —NHCH₃, and —N(CH₃)₂. A subembodiment of this aspect of the disclosure is realized when R³ is selected from hydrogen, C₁₋₆ alkyl, CH₂OCH₃, —OH, —CH₂OH, —CH₂F, —CHF₂, —CF₃, and —NH₂. Another subembodiment of this aspect of the disclosure is realized when R³ is hydrogen. Another subembodiment of this aspect of the disclosure is realized when R³ is C₁₋₆ alkyl selected from —CH₃, —CH₂CH₃, and —CH₂CH(CH₃)₂. Another subembodiment of this aspect of the disclosure is realized when R³ is —CH₂OCH₃. Another subembodiment of this aspect of the disclosure is realized when R³ is —OH or —CH₂OH. Another subembodiment of this aspect of the disclosure is realized when R³ is —CH₂F, —CHF₂, or —CF₃. Still another subembodiment of this aspect of the disclosure is realized when R³ is NH₂. Another subembodiment of this aspect of the disclosure is realized when any hydrogen atom in an alkyl containing substituent of R³ is not deuterated.

Another embodiment of this disclosure is realized when both R² and R³ are hydrogen.

Still another embodiment of the disclosure is realized when at least one of R² and R³ is —OH, or —(CH₂)ₙOH where n is not 0. An aspect of this disclosure is realized when one of R² and R³ is —OH or —(CH₂)ₙOH, where n is not 0. Another aspect of this disclosure is realized when one of R² and R³ is —OH and the other is not. Yet another aspect of this disclosure is realized when one of R² and R³ is —OH and the other is selected from hydrogen and C₁₋₆ alkyl. Still another aspect of this disclosure is realized when one of R² and R³ is —CH₂OH and the other is not. Yet another aspect of this disclosure is realized when one of R² and R³ is —CH₂OH and the other is selected from hydrogen and C₁₋₆alkyl. Still another aspect of this disclosure is realized when one or the other of R² and R³ contains a C₁₋₆alkyl and no hydrogen in said alkyl is deuterated.

Another embodiment of the disclosure is realized when at least one of R² and R³ is —(CH₂)ₙNH₂, —NHCH₃, or —N(CH₃)₂. An aspect of this embodiment of the disclosure is realized when at least one of R² and R³ is (CH₂)ₙNH₂. Still another aspect is realized when one of R² and R³ is NH₂ and the other is not. Yet another aspect of this disclosure is realized when one of R² and R³ is NH₂ and the other is selected from hydrogen, CH₂phenyl and C₁₋₆alkyl. Another aspect is realized when one of R² and R³ is CH₂NH₂ and the other is not. Yet another aspect of this disclosure is realized when one of R² and R³ is CH₂NH₂ and the other is selected from hydrogen, CH₂phenyl and C₁₋₆alkyl. Still another aspect of this disclosure is realized when one or the other of R² and R³ contains C₁₋₆ alkyl and no hydrogen in said alkyl is deuterated.

Another embodiment of the disclosure is realized when R⁴ is selected from C₁₋₆ alkyl, —C₁₋₆ alkylOH, OH, —CH(OH)C₁₋₆ alkyl, —(CH₂)ₙOC₁₋₃ alkyl, —C₃₋₆cycloalkyl, —C₁₋₉ haloalkyl, —(CH₂)ₙOC₁₋₉ haloalkyl, halogen, —CR^{x}R^{y}C₁₋₆ alkylOH, —(CH₂)ₙNH₂, —NHC₁₋₆ alkyl, —N(C₁₋₆ alkyl)₂, and —NHC(O)C₁₋₆alkylNH₂. An aspect of this embodiment of the disclosure is realized when R⁴ is selected from C₁₋₆ alkyl, —(CH₂)ₙOH, —CH(CH₃)OH, —CH₂F, —CHF₂, —CF₃, fluorine, (CH₂)ₙOCHF₂, cyclopropyl, —(CH₂)ₙNH₂, -spirocyclopropylCH₂OH, and —NHC(O)CH(CH₃)NH₂. Another aspect of this disclosure is realized when R⁴ is selected from CH₃, —(CH₂)ₙOH or —CH(CH₃)OH. Another embodiment of this aspect of the disclosure is realized when R⁴ is —CH₃. Another embodiment of this aspect of the disclosure is realized when R⁴ is —(CH₂)ₙOH. Another embodiment of this aspect of the disclosure is realized when R⁴ is —(CH₂)₂OH or —CH₂OH. Another embodiment of this aspect of the disclosure is realized when R⁴ is —(CH₂)₂OH. Another embodiment of this aspect of the disclosure is realized when R⁴ is —CH₂OH. Another embodiment of this aspect of the disclosure is realized when R⁴ is —CH(CH₃)OH. Another embodiment of this aspect of the disclosure is realized when R⁴ is —CH₂F, —CHF₂, or —CF₃. Another embodiment of this aspect of the disclosure is realized when R⁴ is —(CH₂)ₙNH₂. Another embodiment of this aspect of the disclosure is realized when R⁴ is NH₂. Another embodiment of this aspect of the disclosure is realized when R⁴ is NHC(O)CH(CH₃)NH₂. Another embodiment of this aspect of the disclosure is realized when R⁴ is —CR^{x}R^{y}C₁₋₆ alkylOH, wherein R^{x} and R^{y} are alkylene substituents that combine to form cyclopropyl or spirocyclopropyl. Another embodiment of this aspect of the disclosure is realized when R⁴ is -spirocyclopropylCH₂OH. Another embodiment of this aspect of the disclosure is realized when R⁴ is —(CH₂)ₙOCHF₂ or CHF₂. Still another aspect of this disclosure is realized when R⁴ contains an C₁₋₆ alkyl and no hydrogen in said alkyl is deuterated.

Another embodiment of the disclosure is realized when one of R² and R³ is —OH or —(CH₂)ₙOH, where n is not 0, and R⁴ is selected from C₁₋₆ alkyl, —C₁₋₆ alkylOH, —(CH₂)ₙOC₁₋₃alkyl, —CH(OH)C₁₋₆ alkyl, —C₃₋₆cycloalkyl, —C₁₋₉haloalkyl, —(CH₂)ₙOC₁₋₉haloalkyl, halogen, —CR^{x}R^{y}C₁₋₆ alkylOH, —(CH₂)ₙNH₂, —NHC₁₋₆ alkyl, —N(C₁₋₆ alkyl)², and —NHC(O)C₁₋₆alkylNH₂. Yet another aspect of this disclosure is realized when one of R² and R³ is —OH or —CH₂OH, the other is selected from hydrogen and C₁₋₆alkyl and R⁴ is selected from CH₃, —(CH₂)ₙOH, —CH(CH₃)OH, —CH₂F, —CHF₂, —CF₃, fluorine, (CH₂)ₙOCHF₂, cyclopropyl, —(CH₂)ₙNH₂, and -spirocyclopropylCH₂OH, —NHC(O)CH(CH₃)NH₂. An embodiment of this aspect of the disclosure is realized when one of R² and R³ is —OH or —(CH₂)ₙOH, where n is not 0, the other is selected from hydrogen and C₁₋₆alkyl and R⁴ is selected from CH₃, —(CH₂)₂OH, —CH₂OH, —CH(CH₃)OH, —CH₂F, —CHF₂, —CF₃, fluorine, and —(CH₂)ₙNH₂. Another embodiment of this aspect of the disclosure is realized when one of R² and R³ is —OH or —(CH₂)ₙOH where n is not 0, the other is selected from hydrogen and C₁₋₆alkyl and R⁴ is selected from CH₃, —(CH₂)₂OH, —CH₂OH, and —(CH₂)ₙNH₂. An aspect of this disclosure is realized when n is 0, 1, or 2. Still another aspect of this disclosure is realized when R², R³ and/or R⁴ contains an C₁₋₆alkyl and no hydrogen in said alkyl is deuterated.

Another embodiment of the disclosure is realized when one of R² and R³ is one of R² and R³ is —(CH₂)ₙNH₂, —NHCH₃, or —N(CH₃)₂ and the other is selected from hydrogen, CH₂phenyl and C₁₋₆alkyl and R⁴ is selected from C₁₋₆ alkyl, —C₁₋₆ alkylOH, —CH(OH)C₁₋₆ alkyl, —(CH₂)ₙOC₁₋₃alkyl, —C₃₋₆cycloalkyl, —C₁₋₉haloalkyl, —(CH₂)ₙOC₁₋₉haloalkyl, halogen, —CR^{x}R^{y}C₁₋₆ alkylOH, —(CH₂)ₙNH₂, —NHC₁₋₆ alkyl, —N(C₁₋₆ alkyl)², and —NHC(O)C₁₋₆alkylNH₂. Yet another aspect of this disclosure is realized when one of R² and R³ is (CH₂)ₙNH₂, —NHCH₃, or —N(CH₃)₂ and the other is selected from hydrogen, CH₂phenyl and C₁₋₆alkyl and R⁴ is selected from CH₃, —(CH₂)ₙOH, —CH(CH₃)OH, —CH₂F, —CHF₂, —CF₃, fluorine, (CH₂)ₙOCHF₂, cyclopropyl, —(CH₂)ₙNH₂, and -spirocyclopropylCH₂OH, —NHC(O)CH(CH₃)NH₂. An embodiment of this aspect of the disclosure is realized when one of R² and R³ is (CH₂)ₙNH₂, —NHCH₃, or —N(CH₃)₂ and the other is selected from hydrogen, CH₂phenyl and C₁₋₆alkyl and R⁴ is selected from CH₃, —(CH₂)ₙOH, —CH(CH₃)OH, —CH₂F, —CHF₂, —CF₃, fluorine, and —(CH₂)ₙNH₂. Another embodiment of this aspect of the disclosure is realized when one of R² and R³ is (CH₂)ₙNH₂, —NHCH₃, or —N(CH₃)² and the other is selected from hydrogen, CH₂phenyl and C₁₋₆alkyl and R⁴ is selected from —(CH₂)ₙOH, or —(CH₂)ₙNH₂. An aspect of this disclosure is realized when R⁴ is —(CH₂)₂OH. Another aspect of this disclosure is realized when R⁴ is CH₂OH. Another aspect of this disclosure is realized when R⁴ is (CH₂)ₙNH₂. Still another aspect of this disclosure is realized when R², R³ and/or R⁴ contains an C₁₋₆alkyl and no hydrogen in said alkyl is deuterated.

Another embodiment of this disclosure is realized when both R² and R³ are hydrogen and R⁴ is selected from CH₃, —(CH₂)ₙOH, —CH(CH₃)OH, —CH₂F, —CHF₂, —CF₃, fluorine, (CH₂)ₙOCHF₂, cyclopropyl, —(CH₂)ₙNH₂, and -spirocyclopropylCH₂OH, —NHC(O)CH(CH₃)NH².

Another embodiment of the disclosure of Formula I and Formula I′ is represented by structural Formula II.

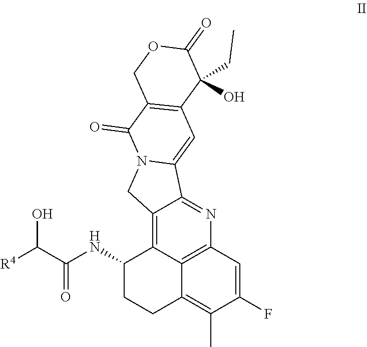

or a pharmaceutically acceptable salt or solvate thereof, wherein R⁴ is as described herein. A subembodiment of the disclosure of Formula II is realized when R⁴ is selected from —CH₃, —OH, —CH₂OH, —(CH₂)₂OH, —CH(CH₃)OH, (CH₂)₂OCHF₂, —C(CH₃)²CH₂OH, —CH₂F, —CHF₂, —CF₃, fluorine, and —(CH₂)ₙNH₂. A subembodiment of the disclosure of Formula II is realized when R⁴ is —CH₃. A subembodiment of the disclosure of Formula II is realized when R⁴ is —OH. A subembodiment of the disclosure of Formula II is realized when R⁴ is —CH₂OH, or —(CH₂)₂OH. A subembodiment of the disclosure of Formula II is realized when R⁴ is —CH(CH₃)OH. Another subembodiment of the disclosure of Formula II is realized when R⁴ is (CH₂)₂OCHF₂. Another subembodiment of the disclosure of Formula II is realized when R⁴ is —C(CH₃)²CH₂OH. Another subembodiment of the disclosure of Formula II is realized when R⁴ is —CH₂F, —CHF₂, or —CF₃. A subembodiment of the disclosure of Formula II is realized when R⁴ is fluorine. A subembodiment of the disclosure of Formula II is realized when R⁴ is —(CH₂)ₙNH₂. Another subembodiment of the disclosure of Formula II is realized when R⁴ contains an C₁₋₆alkyl and no hydrogen in said alkyl is deuterated.

Another embodiment of the disclosure of Formula I and Formula I′ is represented by structural Formula III:

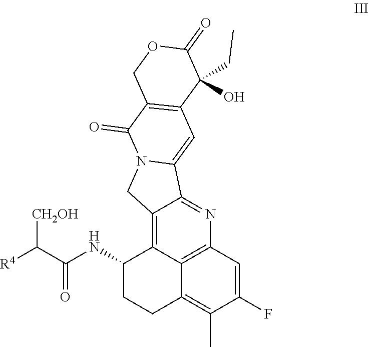

or a pharmaceutically acceptable salt or solvate thereof, wherein R⁴ is as described herein. A subembodiment of the disclosure of Formula III is realized when R⁴ is selected from CH₃, —CH₂OH, —(CH₂)₂OH, —CH(CH₃)OH, (CH₂)₂OCHF₂, —C(CH₃)₂CH₂OH, halogen, —CH₂F, —CHF₂, —OH, —CF₃, and —(CH₂)ₙNH₂. A subembodiment of the disclosure of Formula III is realized when R⁴ is —CH₃. A subembodiment of the disclosure of Formula II is realized when R⁴ is —OH. A subembodiment of the disclosure of Formula III is realized when R⁴ is —CH₂OH, or —(CH₂)₂OH. A subembodiment of the disclosure of Formula III is realized when R⁴ is —CH(CH₃)OH. Another subembodiment of the disclosure of Formula III is realized when R⁴ is (CH₂)₂OCHF₂. Another subembodiment of the disclosure of Formula III is realized when R⁴ is —C(CH₃)₂CH₂OH. Another subembodiment of the disclosure of Formula III is realized when R⁴ is —CH₂F, —CHF₂, or —CF₃. Another subembodiment of the disclosure of Formula III is realized when R⁴ is fluorine. A subembodiment of the disclosure of Formula III is realized when R⁴ is —(CH₂)ₙNH₂. Another subembodiment of the disclosure of Formula III is realized when R⁴ contains an C₁₋₆alkyl and no hydrogen in said alkyl is deuterated.

Another embodiment of the disclosure of Formula I and Formula I′ is represented by structural Formula IV:

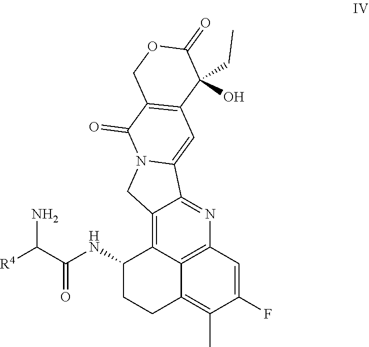

or a pharmaceutically acceptable salt or solvate thereof, wherein R⁴ is as described herein. A subembodiment of the disclosure of Formula IV is realized when R⁴ is OH, —CH₂F, —CHF₂, —CF₃, fluorine, or (CH₂)ₙNH₂.

Another embodiment of the disclosure of Formula I and Formula I′ is represented by structural Formula V:

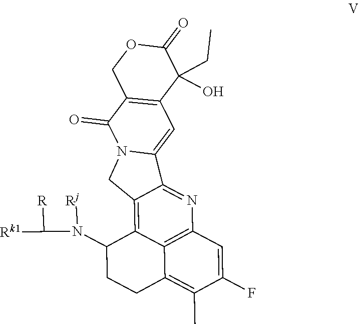

or a pharmaceutically acceptable salt or solvate thereof, wherein R^{j} is as described herein. A subembodiment of the disclosure of Formula V is realized when R^{j} is C₁₋₆ alkyl, said alkyl optionally substituted with 1 to 10 groups of halogen.

Another embodiment of Formula V is realized when R is hydrogen or —C₁₋₆ alkyl.

Another embodiment of Formula V is realized when R^{kl} is —C₁₋₆ alkyl, said alkyl optionally substituted to 1 to 10 halogen, and/or 1 to 3 groups selected from hydroxyl and —C₁₋₆ alkylOH. A subembodiment of this aspect of the invention is realized when R^{kl} is —C₁₋₆ alkyl, said alkyl substituted with 1 to 3 groups selected from hydroxyl and —C₁₋₆ alkylOH, and/or 1 to 10 halogen.

Another embodiment of this disclosure is realized when R^{kl} is (CH₂)ₙC(O)NHC₁₋₆ alkyl, said alkyl optionally substituted to 1 to 10 halogen and/or 1 to 3 groups selected from hydroxyl, and —C₁₋₆ alkylOH.

Another embodiment of this disclosure is realized when R^{kl} is (CH₂)ₙC₆₋₁₀ aryl, said aryl optionally substituted with 1 to 3 groups selected from hydroxyl, and —C₁₋₆ alkylOH.

An aspect of this disclosure relates to a pharmaceutical composition comprising a therapeutically effective amount of a compound of Formula I, I′, II, III, IV, V or a pharmaceutically acceptable salt or solvate thereof and one or more pharmaceutically acceptable carrier(s), diluent(s) or excipients(s).

Another aspect of the disclosure relates to a pharmaceutical composition comprising a therapeutically effective amount of a compound of Formula I, I′, II, III, IV, or V as described herein, or a tautomer, mesomere, racemate, enantiomer, diastereomer thereof, or mixture thereof, or a pharmaceutically acceptable salt thereof and one or more pharmaceutically acceptable carrier(s), diluent(s) or excipient(s).

Another aspect of the disclosure relates to a compound of Formula I, I′, II, III, IV, or V as described herein, or a tautomer, mesomere, racemate, enantiomer, diastereomer thereof, or mixture thereof, or a pharmaceutically acceptable salt thereof for use as a drug or drug component.

Another aspect of the disclosure relates to a compound of Formula I, I′, II, III, IV, or V as described herein, or a tautomer, mesomere, racemate, enantiomer, diastereomer thereof, or mixture thereof, or a pharmaceutically acceptable salt thereof, or a pharmaceutical composition in the preparation of a medicament for treating or preventing a tumor.

In another embodiment, the compounds of the disclosure include those identified herein as Examples in the tables below, and pharmaceutically acceptable salts thereof.

### DETAILED DESCRIPTION OF THE DISCLOSURE

The compounds of the disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all the possible optical isomers and diastereomers in mixtures and as pure or partially purified compounds are included within the ambit of this disclosure. Unless a specific stereochemistry is indicated, the present disclosure is meant to encompass all such isomeric forms of these compounds.

The independent syntheses of these diastereomers or their chromatographic separations may be achieved as known in the art by appropriate modification of the methodology disclosed herein. Their absolute stereochemistry may be determined, amongst other methods, by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of Formulae I, I′, II, III, IV or V the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure may include all suitable isotopic variations of the compounds of generic Formulae I, I′, II, III, IV or V. For example, different isotopic forms of hydrogen (H) include protium (H) and deuterium (H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. For purposes of this invention when a compound is said to be “not deuterated” it means not enriched in deuterium beyond the background state. Isotopically-enriched compounds within generic Formulae I, I′, II, III, IV, or V can be prepared without undue experimentation by conventional techniques well known to those skilled in the art or by processes analogous to those described in the Schemes and Examples herein using appropriate isotopically-enriched reagents and/or intermediates.

When a compound of the disclosure can form tautomers, all such tautomeric forms are also included within the scope of the present disclosure. For example, compounds including carbonyl —CH₂C(O)— groups (keto forms) may undergo tautomerism to form hydroxyl —CH═C(OH)— groups (enol forms). Both keto and enol forms, where present, are included within the scope of the present disclosure.

When any variable (e.g., R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant disclosure in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-element and Si-element bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant disclosure can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

Absolute stereochemistry is illustrated by the use of hashed and solid wedge bonds. As shown in Illus-I and Illus-II. Accordingly, the methyl group of Illus-I is emerging from the page of the paper and the ethyl group in Illus-II is descending into the page, where the cyclohexene ring resides within the plane of the paper. It is assumed that the hydrogen on the same carbon as the methyl group of Illus-I descends into the page and the hydrogen on the same carbon as the ethyl group of Illus-II emerges from the page. The convention is the same where both a hashed and solid rectangle are appended to the same carbon as in Illus-III, the methyl group is emerging from the plane of the paper and the ethyl group is descending into the plane of the paper with the cyclohexene ring in the plane of the paper.

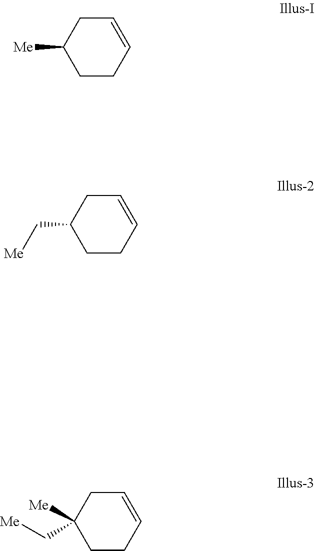 [SMILES: C[C@H]1CC=CCC1 ; CC[C@@H]1CC=CCC1 ; CC[C@]1(C)CC=CCC1]

As is conventional, unless otherwise noted in accompanying text, ordinary “stick” bonds or “wavy” bonds indicate that all possible stereochemistry is represented, including, pure compounds, mixtures of isomers, and racemic mixtures.

As used herein, unless otherwise specified, the following terms have the following meanings:

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”;

“Effective amount” or “therapeutically effective amount” is meant to describe the provision of an amount of at least one compound of the disclosure or of a composition comprising at least one compound of the disclosure which is effective in treating or inhibiting a disease or condition described herein, and thus produce the desired therapeutic, ameliorative, inhibitory or preventative effect. For example, in treating central nervous system diseases or disorders with one or more of the compounds described herein “effective amount” (or “therapeutically effective amount”) means, for example, providing the amount of at least one compound of Formula I, Formula I′, Formula II, Formula III, Formula IV or Formula V that results in a therapeutic response in a patient afflicted with a central nervous system disease or disorder (“condition”), including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition;

“Patient” and “subject” means an animal, such as a mammal (e.g., a human being) and is preferably a human being;

“Prodrug” means compounds that are rapidly transformed, for example, by hydrolysis in blood, in vivo to the parent compound, e.g., conversion of a prodrug of Formula I through Formula V to a compound of Formula I, Formula I′, Formula II, Formula III, Formula IV, or Formula V or to a salt thereof, a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this disclosure includes prodrugs of the novel compounds of this disclosure;

The term “substituted” means that one or more of the enumerated substituents can occupy one or more of the bonding positions on the substrate typically occupied by “—H”, provided that such substitution does not exceed the normal valency rules for the atom in the bonding configuration presented in the substrate, and that the substitution ultimately provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution of a moiety is described (e.g., “optionally substituted”) the term means that if substituents are present, one or more of the enumerated substituents for the specified substrate can be present on the substrate in a bonding position normally occupied by the default substituent normally occupying that position. For example, a default substituent on the carbon atoms of an alkyl moiety is a hydrogen atom, an optional substituent can replace the default substituent.

As used herein, unless otherwise specified, the following terms used to describe moieties, whether comprising the entire definition of a variable portion of a structural representation of a compound of the disclosure or a substituent appended to a variable portion of a structural representation of a group of compounds of the disclosure have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, an “acyl” substituent may be equivalently described herein by the term “acyl”, by typographical representations “R′—(C═O)—” or “R′—C(O)—”, or by a structural representation:

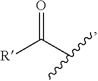

equally, with no differentiation implied using any or all of these representations;

The term “alkyl” (including the alkyl portions of other moieties, such as trifluoromethyl-alkyl- and alkoxy-) means a straight or branched aliphatic hydrocarbon moiety comprising up to about 20 carbon atoms (for example, a designation of “C₁₋₂₀-alkyl” indicates an aliphatic hydrocarbon moiety of from 1 to 20 carbon atoms). In some embodiments, alkyls preferably comprise up to about 10 carbon atoms, unless the term is modified by an indication that a shorter chain is contemplated, for example, an alkyl moiety of from 1 up to 8 carbon atoms is designated herein “C₁₋₈-alkyl”. Where the term “alkyl” is indicated with two hyphens (i.e., “-alkyl-” it indicates that the alkyl moiety is bonded in a manner that the alkyl moiety connects the substituents on either side of it, for example, “-alkyl-OH” indicates an alkyl moiety connecting a hydroxyl moiety to a substrate.

The term “cycloalkyl” means a moiety having a main hydrocarbon chain forming a mono- or bicyclo-cyclic aliphatic moiety comprising at least 3 carbon atoms (the minimum number necessary to provide a monocyclic moiety) up to the maximum number of specified carbon atoms, generally 8 for a monocyclic moiety and 10 for a bicyclic moiety, inclusive of spirocyclic moieties. Examples of cycloalkyl moieties include, but are not limited to, cyclopropyl, cyclobutyl, cyclopentyl or cyclohexyl. The term “cycloalkyl” also includes non-aromatic, fused multicyclic ring system comprising up to 20 carbon atoms which may optionally be substituted as defined herein for “alkyl” generally. Suitable multicyclic cycloalkyls are, for example, but are not limited to: 1-decalin; norbornyl; adamantly; and the like;

As used herein, the term “alkylene” refers to a saturated linear or branched aliphatic hydrocarbon group having two residues derived from the removal of two hydrogen atoms from the same carbon atom or two different carbon atoms of the parent alkane. The alkylene is a linear or branched group having 1 to 20 carbon atoms, preferably 1 to 12 carbon atoms, and more preferably 1 to 6 carbon atoms. Non-limiting examples are methylene, ethylene, propylene, butylene, pentylene, and the like.

As used herein, when the term “alkyl” is modified by “substituted” or “optionally substituted”, it means that one or more C—H bonds in the alkyl moiety group is substituted, or optionally may be substituted, by a substituent bonded to the alkyl substrate which is called out in defining the moiety.

The term “cycloalkyl” refers to a saturated or partially unsaturated monocyclic or polycyclic hydrocarbon substituent group having 3 to 20 carbon atoms, preferably 3 to 12 carbon atoms, more preferably 3 to 10 carbon atoms, and most preferably 3 to 8 carbon atoms. Non-limiting examples of monocyclic cycloalkyl include cyclopropyl, cyclobutyl, cyclopentyl, cyclopentenyl, cyclohexyl, cyclo-hexenyl, cyclohexadienyl, cycloheptyl, cycloheptatrienyl, cyclooctyl and the like. Polycyclic cycloalkyl includes a cycloalkyl having a spiro ring, fused ring or bridged ring.

Where a structural formula represents bonding between a moiety and a substrate using a bonding line that terminates in the middle of the structure, for example the following representations:

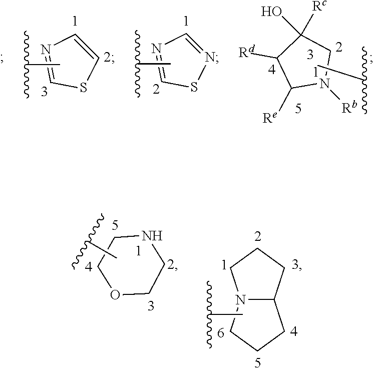

whether or not numbered the structure indicates that unless otherwise defined the moiety may be bonded to the substrate through any of available ring atom, for example, the numbered atoms of the example moieties.

The term “aryl” refers to a 6 to 14 membered all-carbon monocyclic ring or polycyclic fused ring (i.e., each ring in the system shares an adjacent pair of carbon atoms with another ring in the system) having a conjugated it-electron system, preferably a 6 to 10 membered aryl, for example, phenyl and naphthyl, and preferably phenyl.

The term “heteroaryl” refers to an aromatic 5-8 membered monocyclic, 8-12 membered bicyclic, or 11-14 membered tricyclic ring system having 1-3 heteroatoms for monocyclic, 1-6heteroatoms for bicyclic, or 1-9 heteroatoms for tricyclic, said heteroatoms selected from O, N, or S (e.g., carbon atoms and 1-3, 1-6, or 1-9 heteroatoms of N, O, or S for monocyclic, bicyclic, or tricyclic, respectively). Non-limiting examples of heteroaryls are pyridyl, pyrazolyl, pyrimidinyl, furanyl, oxazolyl, triazolyl, oxadiazolyl, and thiophenyl. The heteroaryl groups herein described may also contain fused rings that share a common carbon-carbon bond.

The term “heterocyclyl” (or heterocycloalkyl) means a non-aromatic saturated monocyclic or multicyclic ring system comprising 3 to 10 ring atoms, preferably 5 to 10 ring atoms, in which one or more of the atoms in the ring system is an element other than carbon, for example nitrogen (e.g. piperidyl- or pyrrolidinyl), oxygen (e.g. furanyl and tetrahydropyranyl) or sulfur (e.g. tetrahydrothiopheneyl and tetrahydrothiopyranyl); and wherein the heteroatoms can be alone or in combination provided that the moiety does not contain adjacent oxygen and/or sulfur atoms present in the ring system; preferred heterocyclyl moieties contain 5 to 6 ring atoms; the prefix aza, oxa or thia before the heterocyclyl root name means that at least one nitrogen, oxygen or sulfur atom, respectively, is present as a ring atom; the heterocyclyl can be optionally substituted by one or more independently selected substituents;

The nitrogen or sulfur atom of the heterocyclyl can be optionally oxidized to the corresponding N-oxide, S-oxide or S,S-dioxide (SO₂); non-limiting examples of suitable monocyclic heterocyclyl rings include piperidyl, pyrrolidinyl, piperazinyl, morpholinyl—

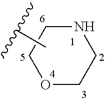

(where unless otherwise noted the moiety is bonded to the substrate through any of ring carbon atoms C2, C3, C5, or C6), thiomorpholinyl, thiazolidinyl, 1,3-dioxolanyl, 1,4-dioxanyl, tetrahydrofuranyl, tetrahydrothiophenyl, tetrahydrothiopyranyl, and the like; and polycyclicheterocyclyl compounds, for example, moieties of the structure:

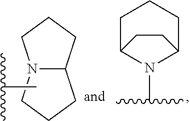

and the like.

The term “solvate” refers to a pharmaceutically acceptable solvate formed by a compound of the present disclosure with one or more solvent molecule(s). Non-limiting examples of solvent molecules include water, ethanol, acetonitrile, isopropanol, DMSO, ethyl acetate.

The term “halogen” means fluorine, chlorine, bromine, or iodine; preferred halogens, unless specified otherwise where the term is used, are fluorine, chlorine and bromine, a substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group, perhaloalkyl (or “fully halogenated” alkyl) means that all bonding positions not participating in bonding the alkyl substituent to a substrate are occupied by a halogen, for example, where the alkyl is selected to be methyl, the term perfluoroalkyl means —CF₃;

The term “hydroxyl” and “hydroxy” means an HO— group, “hydroxyalkyl” means a substituent of the formula: “HO-alkyl-”, wherein the alkyl group is bonded to the substrate and may be substituted or unsubstituted as defined above; preferred hydroxyalkyl moieties comprise a lower alkyl; Non-limiting examples of suitable hydroxyalkyl groups include hydroxymethyl and 2-hydroxyethyl; and The bonding sequence is indicated by hyphens where moieties are represented in text, for example -alkyl, indicates a single bond between a substrate and an alkyl moiety, -alkyl-X, indicates that an alkyl group bonds an “X” substituent to a substrate, and in structural representation, bonding sequence is indicated by a wavy line terminating a bond representation, for example:

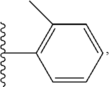 [SMILES: *c1ccccc1C]

indicates that the methylphenyl moiety is bonded to a substrate through a carbon atom ortho to the methyl substituent, while a bond representation terminated with a wavy line and drawn into a structure without any particular indication of an atom to which it is bonded indicates that the moiety may be bonded to a substrate via any of the atoms in the moiety which are available for bonding as described in the examples above.

The line —, as a bond generally indicates a mixture of, or either of, the possible isomers, e.g., containing (R)- and (S)-stereochemical configuration.

Furthermore, unwedged-bolded or unwedged-hashed lines are used in structures containing multiple stereocenters in order to depict relative configuration where it is known. For example:

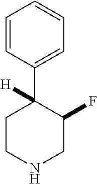 [SMILES: F[C@H]1CNCC[C@@H]1c1ccccc1]

means that the fluorine and hydrogen atoms are on the same face of the piperidine ring, but represents a mixture of, or one of, the possible isomers at right

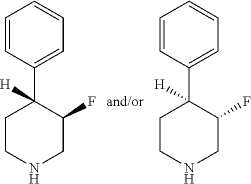

whereas:

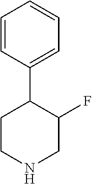 [SMILES: FC1CNCCC1c1ccccc1]

represents a mixture of, or one of, the possible isomers at right

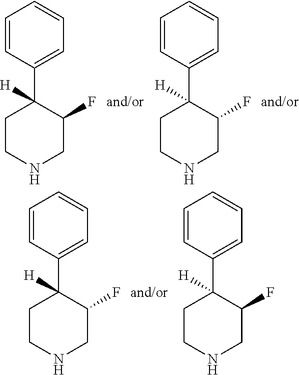

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g. ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

A specific absolute configuration is indicated by use of a wedged-bolded or wedged-hashed line. Unless a specific absolute configuration is indicated, the present disclosure is meant to encompass all such stereoisomeric forms of these compounds.

In this specification, where there are multiple oxygen and/or sulfur atoms in a ring system, there cannot be any adjacent oxygen and/or sulfur present in said ring system.

As well known in the art, a bond drawn from a particular atom wherein no moiety is depicted at the terminal end of the bond indicates a methyl group bound through that bond to the atom, unless stated otherwise. For example:

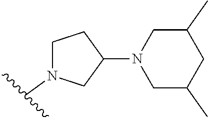 [SMILES: *N1CCC(N2CC(C)CC(C)C2)C1]

represents

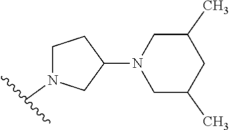 [SMILES: *N1CCC(N2CC(C)CC(C)C2)C1]

Unsatisfied valences in the text, schemes, examples, structural formulae, and any Tables herein is assumed to have a hydrogen atom or atoms of sufficient number to satisfy the valences.

One or more compounds of the disclosure may also exist as, or optionally be converted to, a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

This disclosure also includes the compounds of this disclosure in isolated and purified form obtained by routine techniques. Polymorphic forms of the compounds of Formula I, Formula I′, Formula II, Formula III, Formula IV and Formula V and of the salts, solvates and prodrugs of the compounds of Formula I, Formula I′, Formula II, Formula III, Formula IV and Formula V are intended to be included in the present disclosure. Certain compounds of the disclosure may exist in different isomeric forms (e.g., enantiomers, diastereoisomers, atropisomers). The inventive compounds include all isomeric forms thereof, both in pure form and admixtures of two or more, including racemic mixtures.

In the same manner, unless indicated otherwise, presenting a structural representation of any tautomeric form of a compound which exhibits tautomerism is meant to include all such tautomeric forms of the compound. Accordingly, where compounds of the disclosure, their salts, and solvates and prodrugs thereof, may exist in different tautomeric forms or in equilibrium among such forms, all such forms of the compound are embraced by, and included within the scope of the disclosure. Examples of such tautomers include, but are not limited to, ketone/enol tautomeric forms, imine-enamine tautomeric forms, and for example heteroaromatic forms such as the following moieties:

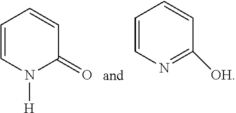 [SMILES: O=c1cccc[nH]1 ; "and" ; Oc1ccccn1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Salts in the solid form may exist in more than one crystal structure and may also be in the form of hydrates. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as formic, hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Salts derived from inorganic bases include aluminum, ammonium, calcium, copper, ferric, ferrous, lithium, magnesium, manganic salts, manganous, potassium, sodium, zinc, and the like.

When the compound of the present disclosure is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the disclosure the salts are citric, hydrobromic, hydrochloric, maleic, phosphoric, sulfuric, fumaric, and tartaric acids. Similarly, the salts of the acidic compounds are formed by reactions with the appropriate inorganic or organic base.

The term “toxic drug” refers to a substance that inhibits or stops the function of cells and/or causes cell death or destruction. Toxic drugs include toxins and other compounds that can be used in tumor treatment.

The term “toxin” refers to any substance that can have a harmful effect on the growth or proliferation of cells. Toxins can be small molecule toxins and their derivatives from bacteria, fungi, plants or animals, including Camptothecin derivatives such as exatecan, maytansinoid and its derivatives (CN101573384) such as DM1, DM3, DM4, auristatin F (AF) and its derivatives such as MMAF, MMAE, 3024 (WO 2016/127790 A1, compound 7), diphtheria toxin, exotoxin, ricin A chain, abrin A chain, modeccin, a-sarcin, Aleutites fordii toxic protein, dianthin toxic protein, Phytolaca americana toxic protein, Momordica charantia inhibitor, curcin, crotin, Sapaonaria ojficinalis inhibitor, gelonin, mitogellin, restrictocin, phenomycin, enomycin and trichothecenes.

The term “chemotherapeutic drug” refers to a chemical compound that can be used to treat tumors. This definition also includes antihormonal agents that act to modulate, reduce, block, or inhibit the effects of hormones that promote cancer growth, which are often in the form of systemic or holistic therapy. They can be hormones. Examples of chemotherapeutic drugs include alkylating agents, such as thiotepa; cyclosphamide (CYTOXANTM); alkyl sulfonate such as busulfan, improsulfan and piposul-fan; aziridine such as benaodopa, carboquone, meturedopa and uredopa; aziridine and methylamelamine including altretamine, triethy lenemelamine, triethy lenephosphor-amide, triethylenethiophosphoramide and trimethylolomela-mine; nitrogen mustards such as chlorambucil, chlornaphaz-ine, cholophosphamide, estramustine, ifosfamide, mechlorethamine, nitrobin hydrochloride; melphalan, novembichin, phenesterine, prednimustine, trofosfamide, uramustine; nitrosureas such as carmustine, chlorozotocin, fotemustine, lomustine, nimustine, ranimustine; antibiotic such as aclacinomycin, actinomycin, authramycin, azaser-ine, bleomycin, cactinomycin C, calicheamicin, carabicin, chromomycin, carzinophilin, chromomycin, actinomycin D, daunorubicin, detorubicin, 6-diazo-5-oxy-L-norleucine, doxorubicin, epirubicin, esorubicin, idarubicin, marcello-mycin, mitomycin, mycophenolic acid, nogalamycin, olivo-mycin, peplomycin, potfiromycin, puromycin, quelamycin, rodorubicin, streptonigrin; streptozocin, tuberculocidin, ubenimex, zinostatin, zorubicin; antimetabolites such as methotrexate, 5-fluorouracil (5-FU); folic acid analogs such as denopterin, methotrexate, pteropterin, trimetrexate; pterin analogs such as fludarabine, 6-mercaptopterin, thiomethop-terin, thioguanopterin; pyrimidine analogs such as ancit-abine, azacitidine, 6-azuridine, carmofur, cytarabine, dide-oxyuridine, doxitluridine, enocitabine, floxuridine, 5-FU; androgens such as calusterone, dromostanolong propionate, epitiostanol, mepitiostane, testolactone; anti-adrenalines such as aminoglutethimide, mitotane, trilostane; folic acid supplements such as frolinic acid; aceglatone; aldophosph-amideglycoside; aminolevulinic acid; amsacrine; bestrabu-cil; biasntrene; edatraxate; defofamine; demecolcine; diazi-quone; elfomithine; elliptinium acetate; etoglucid; gallium nitrate; hydroxyurea; lentinan; lonidamine; mitoguazone; mitoxantrone; mopidamol; nitracrine; pintostatin; phe-namet; pirarubicin; podophyllinic acid; 2-ethylhydrazide; procarbazine; PSK®; razoxane; sizofiran; spirogermanium; tenuazonic acid; triaziquone; 2,2′,2″-trichlorrotriethylamine; urethan; vindesine; dacarbazine; mannomustine; mitobroni-tol; dibromodulcitol; pipobroman; gacytosine; arabinoside (“Ara-C”); cyclophosphamide; thiotepa; taxanes such as paclitaxel (TAXOL®, Bristol-Myers Squibb Oncology, Princeton, N.J.) and docetaxel (TAXOTERE®, Rhone-Pou-lenc Rorer, Antony, France); chlorambucil; gemcitabine; 6-thioguanine; mercaptopurine; methotrexate; platinum ana-logs such as cisplatin and carboplatin; vinblastine; platinum; etoposide (VP-16); ifosfamide; mitomycin C; mitoxantrone; vincristine; vinorelbine; navelbine; novantrone; teniposide; daunorubicin; aminopterin; xeloda; ibandronate; CPT-11; topoisomerase inhibitor RFS2000; difluoromethylomithine (DMFO); retinoic acid esperamicins; capecitabine; and pharmaceutically acceptable salt, acid or derivative of any of the above substances. This definition also includes anti-hormonal agents that can modulate or inhibit the effects of hormones on tumors, such as anti-estrogens, including tamoxifen, raloxifene, aromatase inhibitor 4(5)-imidazole, 4-hydroxytamoxifen, trioxifene, keoxifene, LYll 7018, ona-pristone and Fareston; and anti-androgens such as flutamide, nilutamide, bicalutamide, leuprolide and goserelin; and pharmaceutically acceptable salt, acid or derivative of any of the above substances.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof. “Treatment” as used herein also refers to control, amelioration, or reduction of risks to the subject afflicted with a disease, disorder or condition in which a tumor is involved. The terms “preventing” or “prevention” or “prophylaxis” of a disease, disorder or condition as used herein includes: impeding the development or progression of clinical symptoms of the disease, disorder, or condition in a mammal that may be exposed to or predisposed to the disease, disorder or condition but does not yet experience or display symptoms of the disease, and the like.

As would be evident to those skilled in the art, subjects treated by the methods described herein are generally mammals, including humans and non-human animals (e.g., laboratory animals and companion animals). The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising a compound of the disclosure or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the disclosure or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

As noted above, additional embodiments of the present disclosure are each directed to a method for the treatment a disease, disorder, or condition, or one or more symptoms thereof (“indications”) which method comprises administering to a subject in need of such treatment a therapeutically effective amount of a compound of the disclosure, or a pharmaceutically acceptable salt thereof, or a pharmaceutical composition comprising said compound or salt thereof.

In another embodiment, the present disclosure is directed to a method for the manufacture of a medicament for use in a subject comprising combining a compound of the present disclosure, or a pharmaceutically acceptable salt thereof, with a pharmaceutical carrier or diluent.

One such embodiment provides a method of treating or preventing a cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer) colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma) in a subject in need thereof, said method comprising administering to a subject in need of such treatment a therapeutically effective amount of a compound of the disclosure, or a pharmaceutically acceptable salt or solvate thereof, or a pharmaceutical composition comprising said compound, salt or solvate thereof. In one such embodiment, the subject is a human.

Another aspect of the disclosure relates to a method for treating and/or preventing a tumor, comprising administering to a patient in need thereof a therapeutically effective amount of the compound, or a pharmaceutically acceptable salt or solvate thereof, or the pharmaceutical composition comprising the compound according to the present disclosure.

Combinations with additional therapeutic agents are also contemplated in the instant methods. For example, combinations of the compounds of the present disclosure with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. No. 60/235,708 and 60/244,697), or a pharmaceutically acceptable salt thereof.

Another embodiment of the instant disclosure is the use of the compounds of the present disclosure in combination with gene therapy for the treatment of cancer. For an overview of genetic strategies to treating cancer see Hall et al., (Am. J. Hum. Genet. 61:785-789, 1997) and Kufe et al., (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

The compounds of the disclosure may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar), or a pharmaceutically acceptable salt thereof.

The compounds of the present disclosure may also be administered with an immunologic-enhancing drug, such as levamisole, isoprinosine and Zadaxin, or a pharmaceutically acceptable salt thereof.

The compounds of the present disclosure may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir, or a pharmaceutically acceptable salt thereof.

The compounds of the present disclosure may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, Cl1033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR9576, indinavir, amprenavir, cortisol, testosterone, LY335979, OC144-093, erythromycin, vincristine, digoxin and talinolol, or a pharmaceutically acceptable salt thereof.

The compounds of the present disclosure may also be useful for treating or preventing cancer, including bone cancer, in combination with bisphosphonates, including but not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

The compounds of the present disclosure may also be useful for treating or preventing breast cancer in combination with aromatase inhibitors. Examples of aromatase inhibitors include but are not limited to: anastrozole, letrozole and exemestane, or a pharmaceutically acceptable salt thereof.

The compounds of the present disclosure may also be useful for treating or preventing cancer in combination with siRNA therapeutics.

The compounds of the present disclosure may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139), or a pharmaceutically acceptable salt thereof.

In one embodiment, specific anticancer agents useful in the present combination therapies include, but are not limited to: pembrolizumab (Keytruda®), abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); Olaparib (Lynparza®) vorinostat (Zolinza®) and zoledronate (Zometa®), or a pharmaceutically acceptable salt thereof.

Thus, the scope of the instant disclosure encompasses the use of the compounds the present disclosure in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

Yet another example of the disclosure is a method of treating cancer that comprises administering a therapeutically effective amount of a compound of the present disclosure in combination with paclitaxel or trastuzumab.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a compound of the present disclosure is combined with one or more other anti-cancer agents for use in the prevention, treatment, control amelioration, or reduction of risk of a particular disease or condition for which the compounds of the present disclosure are useful. Such other active agents may be administered, by a route and in an amount commonly used therefor, prior to, contemporaneously, or sequentially with a compound of the present disclosure.

The instant disclosure also includes a pharmaceutical composition useful for treating or preventing cancer that comprises a therapeutically effective amount of compounds of the present disclosure and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

The disclosure further relates to a method of treating cancer in a human patient comprising administration of an and a PD-1 antagonist to the patient. The compound of the disclosure and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is an anti-PD-1 antibody, independently selected from pembrolizumab, nivolumab, cemiplimab, sintilimab, tislelizumab, atezolizumab (MPDL3280A), camrelizumab and toripalimab. In other embodiments, the PD-L1 antagonist is an anti-PD-L1 antibody independently selected from atezolizumab, durvalumab and avelumab.

In one embodiments, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

When the compounds of the present disclosure are administered in combination with an anti-human PD-1 antibody (or antigen-binding fragment thereof), the anti-human PD-1 antibody (or antigen-binding fragment thereof) may be administered either simultaneously with, or before or after, the compounds of the present disclosure. Either of the anti-human PD-1 antibody (or antigen-binding fragment thereof), and/or a compound of the present disclosure, or a pharmaceutically acceptable salt thereof, may be administered separately, by the same or different route of administration, or together in the same pharmaceutical composition as the other agent(s). The weight ratio of the anti-human PD-1 antibody (or antigen-binding fragment thereof) to a compound of the present disclosure, may be varied and will depend upon the therapeutically effective dose of each agent. Generally, a therapeutically effective dose of each will be used. Combinations including at least one anti-human PD-1 antibody (or antigen-binding fragment thereof), a compound of the present disclosure, and optionally other active agents will generally include a therapeutically effective dose of each active agent. In such combinations, the anti-human PD-1 antibody (or antigen-binding fragment thereof), the compounds and other active agents may be administered separately or in conjunction. In addition, the administration of one element may be prior to, concurrent with, or subsequent to the administration of other agent(s).

In one embodiment, this disclosure provides an anti-human PD-1 antibody (or antigen-binding fragment thereof), and/or a compound of the disclosure, and at least one other active agent as a combined preparation for simultaneous, separate or sequential use in treating cancer.

The disclosure also provides the use of a compound of the present disclosure, for treating cancer, where the patient has previously (e.g., within 24-hours) been treated with an anti-human PD-1 antibody (or antigen-binding fragment thereof). The disclosure also provides the use of an anti-human PD-1 antibody (or antigen-binding fragment thereof) for treating a cellular proliferative disorder, where the patient has previously (e.g., within 24-hours) been treated with an antibody-linker-payload compound (ADC) a compound of the present disclosure.

The present disclosure further relates to methods of treating cancer, said method comprising administering to a subject in need thereof a combination therapy that comprises (a) a compound of the present disclosure, and (b) an anti-human PD-1 antibody (or antigen-binding fragment thereof); wherein the anti-human PD-1 antibody (or antigen-binding fragment thereof) is administered once every 21 days.

Additionally, the present disclosure relates to methods of treating cancer, said method comprising administering to a subject in need thereof a combination therapy that comprises: (a) a compound of the present disclosure, and (b) an anti-human PD-1 antibody (or antigen-binding fragment thereof. In specific embodiments, the cancer occurs as one or more solid tumors or lymphomas. In further specific embodiments, the cancer is selected from the group consisting of advanced or metastatic solid tumors and lymphomas. In still further specific embodiments, the cancer is selected from the group consisting of malignant melanoma, head and neck squamous cell carcinoma, MSI-H cancer, MMR deficient cancer, non-small cell lung cancer, urothelial carcinoma, gastric or gastroesophageal junction adenocarcinoma, breast adenocarcinoma, and lymphomas. In additional embodiments, the lymphoma is selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma. In particular embodiments, the cellular proliferative disorder is a cancer that has metastasized, for example, a liver metastases from colorectal cancer. In additional embodiments, the cellular proliferative disorder is a cancer is classified as stage III cancer or stage IV cancer. In instances of these embodiments, the cancer is not surgically resectable.

In embodiments of the methods disclosed herein, the anti-human PD-1 antibody (or antigen binding fragment thereof) is administered by intravenous infusion or subcutaneous injection.

In one embodiment, the present disclosure provides compositions comprising a compound of the disclosure, a pharmaceutically acceptable carrier, and an anti-human PD-1 antibody (or antigen-binding fragment thereof).

In another embodiment, the present disclosure provides compositions comprising a compound of the disclosure, a pharmaceutically acceptable carrier, and pembrolizumab.

In one embodiment, the present disclosure provides compositions comprising a compound of the disclosure, a pharmaceutically acceptable carrier, and two additional therapeutic agents, one of which is an anti-human PD-1 antibody (or antigen-binding fragment thereof), and the other of which is independently selected from the group consisting of anticancer agents.

A compound of the present disclosure may be employed in conjunction with anti-emetic agents to treat nausea or emesis, including acute, delayed, late-phase, and anticipatory emesis, which may result from the use of a compound of the present disclosure, alone or with radiation therapy. For the prevention or treatment of emesis, a compound of the present disclosure may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide, aprepitant, fosaprepitant, or dronabinol. In another example, conjunctive therapy with an anti-emesis agent selected from a neurokinin-1 receptor antagonist, a 5HT3 receptor antagonist and a corticosteroid is disclosed for the treatment or prevention of emesis that may result upon administration of the compounds of the disclosure.

The compounds of the disclosure may also be administered with an agent useful in the treatment of anemia. Such an anemia treatment agent is, for example, a continuous erythropoiesis receptor activator (such as epoetin alfa).

The compounds of the disclosure may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The compounds of the disclosure may be useful when co-administered with other treatment modalities, including but not limited to, radiation therapy, surgery, and gene therapy. Accordingly, in one embodiment, the methods of treating cancer described herein, unless stated otherwise, can optionally include the administration of an effective amount of radiation therapy. For radiation therapy, γ-radiation is preferred.

The methods of treating cancers described herein can optionally include the administration of an effective amount of radiation (i.e., the methods of treating cancers described herein optionally include the administration of radiation therapy).

The methods of treating cancer described herein include methods of treating cancer that comprise administering a therapeutically effective amount of a compound of Formula IV in combination with radiation therapy and/or in combination with a second compound selected from: an estrogen receptor modulator, an androgen receptor modulator, a retinoid receptor modulator, a cytotoxic/ytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the additional therapeutic agents listed herein.

Additional embodiments of the disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### Kits

In one aspect, provided is a kit comprising a therapeutically effective amount of a compound of the present disclosure or a pharmaceutically acceptable salt, solvate or ester of said compound and a pharmaceutically acceptable carrier, vehicle or diluent.

In another aspect provided is a kit comprising an amount of a compound of the present disclosure, and an amount of at least one additional therapeutic agent listed above, wherein the amounts of the two or more active ingredients result in a desired therapeutic effect. In one embodiment, the compound of the present disclosure, and the one or more additional therapeutic agents are provided in the same container. In one embodiment, the compound of the present disclosure, and the one or more additional therapeutic agents are provided in separate containers.

The present disclosure includes within its scope prodrugs of the compounds of this disclosure. In general, such prodrugs will be functional derivatives of the compounds of this disclosure which are readily convertible in vivo into the required compound. Thus, in the methods of treatment of the present disclosure, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this disclosure into the biological milieu.

The compounds described herein, or pharmaceutically acceptable salts and/or solvates thereof, may be administered singly, in combination with other compounds of the disclosure, and/or in cocktails combined with other therapeutic agents. The choice of therapeutic agents that can be co-administered with the compounds of the disclosure will depend, in part, on the condition being treated.

The compounds of the present disclosure may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the disclosure are effective for use in humans.

The pharmaceutical compositions for the administration of the compounds of this disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present disclosure are employed. Similarly, transdermal patches may also be used for topical administration.

The pharmaceutical composition and method of the present disclosure may further comprise other therapeutically active compounds as noted herein which are usually applied in the treatment of the above-mentioned pathological conditions.

In the treatment, prevention, control, amelioration, or reduction of risk of the conditions disclosed herein an appropriate dosage level of the compounds of this disclosure will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this disclosure are illustrated in the following Schemes and Examples. Starting materials are made according to procedures known in the art or as illustrated herein.

### PREPARATIVE EXAMPLES

The compounds of the present disclosure can be prepared according to the following schemes and specific examples, or modifications thereof, using readily available starting materials, reagents and conventional synthesis procedures. It is also possible to make use of variants which are themselves known to those of ordinary skill in this art but are not mentioned in detail. The general procedures for making the compounds claimed in this disclosure can be readily understood by one skilled in the art from viewing the following schemes and descriptions. Abbreviations used in the experimentals may include, but are not limited to the following:

| | |
|---|---|
| aq | Aqueous |
| CDI | Carbonyldiimidazole |
| CH₃I | Iodomethane |
| DAST | Diethylaminosulfur trifluoride |
| DCE | 1,2-Dichloroethane |
| DCM | Dichloromethane |
| DIPEA | N,N-Diisopropylethylamine |
| DMAP | 4-Dimethylaminopyridine |
| DMF | Dimethylformamide |
| eq. | Equivalent(s) |
| DMSO | Dimethyl sulfoxide |
| EtOAc | Ethyl acetate |
| Et₃N | Triethylamine |
| hr | Hour(s) |
| ¹H-NMR | Proton nuclear magnetic resonance |
| HCl | Hydrochloric acid |
| HPLC | High performance liquid chromatography |
| IPA | Isopropyl alcohol |
| KHMDS | Potassium bis(trimethylsilyl)amide |
| LCMS | Liquid chromatography-mass spectrometry |
| LiOH | Lithium hydroxide |
| MgSO₄ | Magnesium Sulfate |
| min | Minute(s) |
| MeCN | Acetonitrile |
| MeOH | Methanol |
| MS | Mass spectrometry |
| m/z | Mass to charge ratio |
| Na₂SO₄ | Sodium sulfate |
| NaHCO₃ | Sodium bicarbonate |
| NaBH₄ | Sodium borohydride |
| Pd | Palladium |
| Pd/C | Palladium on carbon |
| RT | Room temperature |
| SFC | Supercritical Fluid Chromatography |
| TBSCl | tert-Butyl dimethylsilyl chloride |
| TFA | Trifluoroacetic acid |
| THF | Tetrahydrofuran |
| TLC | Thin Layer Chromatography |
| t_{R} | Retention time |
| TEA | Triethylamine |
| Celite | Trademark for diatomaceous earth |
| HATU | 1-[Bis(dimethylamino)methylene]-1H-1,2,3-triazolo[4,5- |
| | b]pyridinium 3-oxid hexafluorophosphate |
| DIEA | Diisopropyl ethyl amine |

### General Experimental Information:

Unless otherwise noted, all reactions were magnetically stirred. All reagents and solvents were purchased from commercial sources and used as is unless otherwise noted.

Reaction progress and synthetic intermediate analysis were assessed by LCMS (UV detection with ESI, APCI, or other mass detection) when applicable using a MeCN/water gradient with either TFA, formic acid, or NH₄HCO₃ modifier. Silica gel and reverse-phase flash column chromatography were conducted with commercially available pre-packed columns. Reverse-phase preparative HPLC purification was performed on preparative HPLC instruments with UV and MS detection using a MeCN/water gradient with either TFA, formic acid, or NH₄OH modifier. ¹H NMR spectra were collected at room temperature, and chemical shifts are reported in ppm relative to the residual proteo-solvent signals, and multiplicities, coupling constants (where applicable), and signal integrations are listed parenthetically. Unless otherwise noted, all EC₅₀ data presented in tables refers to the CellTiter-Glo® 2.0 Cytotoxicity Assay that is described in the Biological Assay section.

### SYNTHETIC SCHEMES, INTERMEDIATES, AND EXAMPLES

The compounds of the disclosure may be prepared by methods known in the art of organic synthesis as set forth in part by the following general synthetic schemes and specific preparative examples. Starting materials are available commercially or may be prepared by known methods.

### Example 1

### Preparation of Compound 1

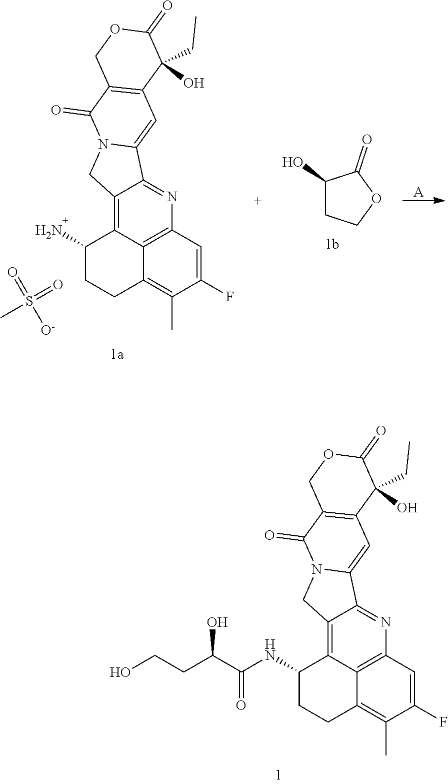

To a solution of (R)-3-hydroxydihydrofuran-2(3H)-one (1b, 200 mg, 2.0 mmol) in MeOH (0.20 mL) and DMF (0.40 mL) was added (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 0.40 mL, 0.12-0.16 mmol, ˜0.35-0.40 M in DMF with Hunig's base) mixture. The reaction was heated to 70° C. over 5 nights. The mixture was cooled to room temperature, filtered through a syringe filter, and then subjected to reverse phase column chromatography (25-60% MeCN/water with 0.1% formic acid modifier) to afford (R)—N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxybutanamide (1) as a solid (Peak 1 desired product, minor isomer product peak 2 also detected). MS: m/z=538 [M+H]. ¹H NMR (500 MHz, DMSO-d₆): δ 8.44 (d, J=9.0 Hz, 1H), 7.76 (d, J=11.0 Hz, 1H), 7.30 (s, 1H), 6.52 (s, 1H), 5.59-5.54 (m, 1H), 5.41 (s, 2H), 5.24-5.07 (m, 2H), 4.11 (dd, J=9.1, 3.4 Hz, 1H), 3.59-3.54 (m, 2H), 3.25-3.08 (m, 3H), 2.38 (s, 3H), 2.23-2.10 (m, 2H), 2.03-1.94 (m, 1H), 1.91-1.74 (m, 3H), 0.87 (t, J=7.3 Hz, 3H).

The following compounds of the present disclosure in Table 1 were made using similar methods described in Example 1 with varying reaction times (2-3 days, and 60-65° C.) and substituting the appropriate reactants and/or reagents:

TABLE 1

| Com- | | MS |
|---|---|---|
| pound | Structure | [M + H] |
| 2 | 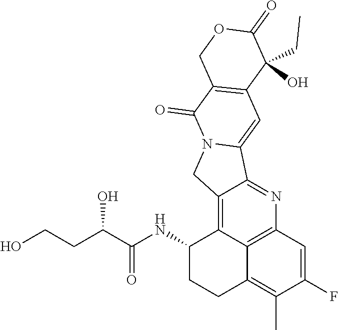 [SMILES: CC[C@@]1(O)C(=O)OCc2c1cc1n(c2=O)Cc2c-1nc1cc(F)c(C)c3c1c2[C@@H](NC(=O)[C@@H](O)CCO)CC3] | 538 |
| 3 | 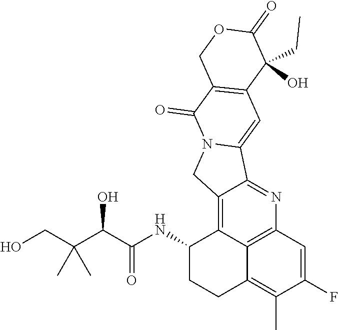 [SMILES: CC[C@@]1(O)C(=O)OCc2c1cc1n(c2=O)Cc2c-1nc1cc(F)c(C)c3c1c2[C@@H](NC(=O)[C@H](O)C(C)(C)CO)CC3] | 566 |

### Intermediate I-1c

### Preparation of Intermediate I-1c

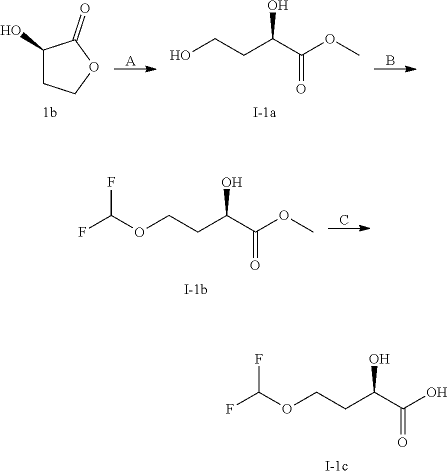

### Step A—Synthesis of Intermediate I-1a

To a solution of (R)-3-hydroxydihydrofuran-2(3H)-one (1b, 2.2 g, 22 mmol) in MeOH (20 mL) was added Dowex 50W X8 (hydrogen form, strongly acidic), 200-400 mesh resin (1.1 g). The reaction was stirred at room temperature for 2 hr 20 min. The mixture was filtered over Celite®, and the filtrate was concentrated under reduced pressure. The resulting methyl (R)-2,4-dihydroxybutanoate (I-1a) was used directly in the next reaction as an oil. ¹H NMR (500 MHz, CDCl₃): δ 4.40 (dd, J=7.8, 3.9 Hz, 1H), 3.91-3.81 (m, 2H), 3.81 (s, 3H), 2.13-2.04 (m, 1H), 1.96-1.88 (m, 1H).

### Step B—Synthesis of Intermediate I-1b

To a mixture of methyl (R)-2,4-dihydroxybutanoate (I-1a, 2.0 g, 15 mmol) and potassium acetate (6.0 g, 61 mmol) in DCM (10 mL) and water (10 mL) was added (bromodifluoromethyl)trimethylsilane (4.0 mL, 26 mmol). The reaction was vigorously stirred at room temperature over three nights. The mixture was diluted with saturated NaHCO₃, DCM, and 3:1 CHCl₃:IPA (100 mL). The resulting mixture was shaken and then passed through a hydrophobic membrane phase separator. The remaining aqueous layer was re-extracted with 3:1 CHCl₃:IPA (100 mL), and that mixture was also passed through a hydrophobic membrane phase separator. The combined organic layer was concentrated under reduced pressure. The crude material was subjected to silica gel column chromatography (0-100% EtOAc/hexanes) to afford methyl (R)-4-(difluoromethoxy)-2-hydroxybutanoate (I-1b) as an oil. ¹H NMR (500 MHz, CDCl₃) δ 6.18 (t, J=74.9 Hz, 1H), 4.35-4.29 (m, 1H), 4.06-3.98 (m, 2H), 3.81 (s, 3H), 2.85 (d, J=4.9 Hz, 1H), 2.20-2.13 (m, 1H), 2.02-1.93 (m, 1H).

### Step C—Synthesis of Intermediate I-1c

To a solution of methyl (R)-4-(difluoromethoxy)-2-hydroxybutanoate (I-1b, 19 mg, 0.10 mmol) in DCE (2.0 mL) was added trimethyltin hydroxide (75 mg, 0.41 mmol). The reaction was stirred at 60° C. overnight. The mixture was cooled, diluted with EtOAc and 1M HCl, and shaken. The organic layer was separated and passed through a hydrophobic membrane phase separator. The organic layer was then concentrated under reduced pressure. The resulting (R)-4-(difluoromethoxy)-2-hydroxybutanoic acid (I-1c) was used crude directly in the next reaction as described in Example 3.

### Intermediate I-2d

### Preparation of Intermediate I-2d

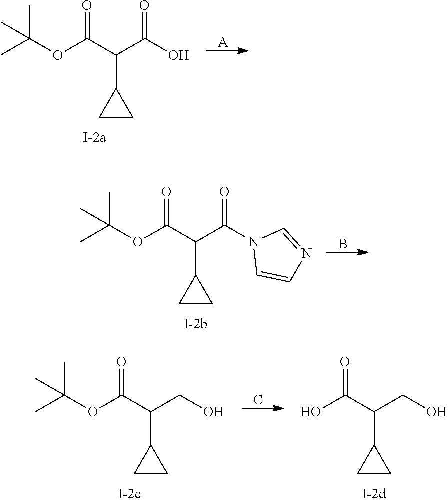

### Step A—Synthesis of Intermediate I-2b

To a mixture of 3-(tert-butoxy)-2-cyclopropyl-3-oxopropanoic acid (I-2a, 0.10 g, 0.50 mmol) and CDI (0.12 g, 0.75 mmol) was added DCM (2.0 mL). The reaction was stirred at room temperature overnight. The reaction was diluted with DCM and water, extracted, and then passed through a hydrophobic membrane phase separator. The organic layer was concentrated under reduced pressure to afford crude tert-butyl 2-cyclopropyl-3-(1H-imidazol-1-yl)-3-oxopropanoate (I-2b), which was used directly in the next reaction. 1H NMR (500 MHz, CDCl₃) δ 8.18 (s, 1H), 7.49 (s, 1H), 7.11 (s, 1H), 3.08 (d, J=9.7 Hz, 1H), 1.60-1.52 (m, 1H), 1.42 (s, 9H), 0.82-0.72 (m, 2H), 0.53-0.46 (m, 1H), 0.30-0.24 (m, 1H).

### Step B—Synthesis of Intermediate I-2c

To a solution of NaBH₄ (0.060 g, 1.5 mmol) in THF (2.0 mL) and water (1.5 mL) was added a solution of tert-butyl 2-cyclopropyl-3-(1H-imidazol-1-yl)-3-oxopropanoate (I-2b, 0.13 g, 0.5 mmol) in THF (2.0 mL) dropwise. The reaction was stirred at RT overnight. The reaction was then diluted with DCM, water, and 1M HCl (3.0 mL). The mixture was extracted and passed through a hydrophobic membrane phase separator. The organic layer was concentrated under reduced pressure to afford crude tert-butyl 2-cyclopropyl-3-hydroxypropanoate (I-2c), which was used directly in the next reaction. ¹H NMR (500 MHz, CDCl₃) δ 3.86-3.78 (m, 2H), 1.74-1.68 (m, 1H), 1.48 (s, 9H), 0.95-0.86 (m, 1H), 0.57-0.52 (m, 2H), 0.40-0.35 (m, 1H), 0.20-0.16 (m, 1H).

### Step C—Synthesis of Intermediate I-2d

To a solution of tert-butyl 2-cyclopropyl-3-hydroxypropanoate (I-2c, 0.090 g, 0.50 mmol) in DCM (4.0 mL) was added HCl (0.40 mL, 1.6 mmol, 4.0 M in dioxane). The reaction was stirred at room temperature overnight. The mixture was concentrated under reduced pressure, and the resulting crude 2-cyclopropyl-3-hydroxypropanoic acid (I-2d) was used directly in the next reaction as described in Example 3.

### Intermediates I-3d and I-3e

### Preparation of Intermediates I-3d and I-3e

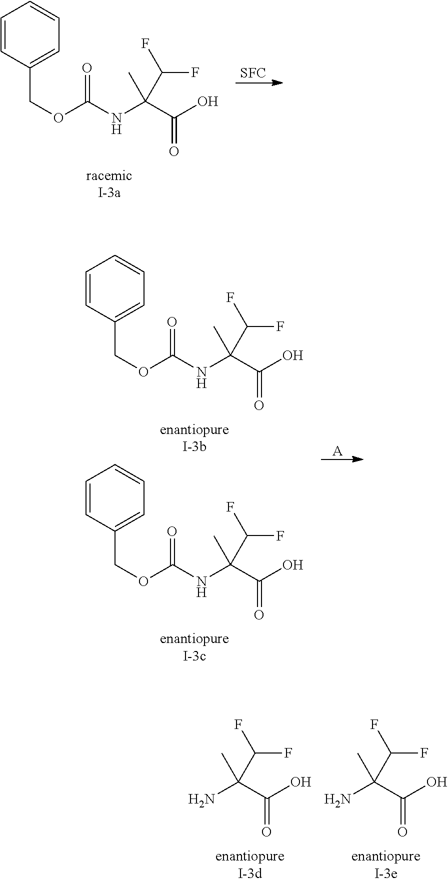

SFC Separation of Enantiomers to Yield I-3b and I-3c

Racemic 2-(((benzyloxy)carbonyl)amino)-3,3-difluoro-2-methylpropanoic acid (I-3a, 1.2 g, 4.4 mmol) was subjected to preparative chiral SFC (AD-H 2×25 cm, 10% (EtOH+0.1% DEA)/CO₂, 100 bar, 70 mL/min) to afford enantiomer 1 (peak 1, 95% ee, absolute configuration unknown) and enantiomer 2 (peak 2, 99% ee, absolute configuration unknown) as diethylamine salts.

To obtain the free acid, the diethylamine salt of enantiomer 1 was dissolved in EtOAc and shaken with aqueous 1M HCl. The organic layer was back-extracted with EtOAc twice. The combined organic layers were dried over Na₂SO₄, filtered, and concentrated under reduced pressure to afford 2-(((benzyloxy)carbonyl)amino)-3,3-difluoro-2-methylpropanoic acid (I-3b, enantiomer 1, absolute configuration unknown). ¹H NMR (500 MHz, DMSO-d₆) δ 13.20 (s, 1H), 8.10 (s, 1H), 7.42-7.27 (m, 5H), 6.28 (t, J=56.1 Hz, 1H), 5.03 (s, 2H), 1.34 (s, 3H).

The free acid of 2-(((benzyloxy)carbonyl)amino)-3,3-difluoro-2-methylpropanoic acid (I-3c, enantiomer 2, absolute configuration unknown) was obtained in analogous fashion.

### Step A—Synthesis of Intermediates I-3d and I-3e

A mixture of 2-(((benzyloxy)carbonyl)amino)-3,3-difluoro-2-methylpropanoic acid (I-3b, enantiomer 1, 290 mg, 1.1 mmol), 10 wt % Pd/C (430 mg, 0.41 mmol Pd) and MeOH (20 mL) was stirred under H₂ (1 atm) overnight. The reaction mixture was then filtered through Celite®, rinsing with MeOH and DCM, followed by removal of solvent under reduced pressure to afford 2-amino-3,3-difluoro-2-methylpropanoic acid (I-3d, enantiomer 1, absolute configuration unknown) as a solid. 1H NMR (500 MHz, DMSO-d₆) δ 7.71 (br s, 3H), 6.18 (t, J=54.4 Hz, 1H), 1.27 (s, 3H).

2-Amino-3,3-difluoro-2-methylpropanoic acid (I-3e, enantiomer 2, absolute configuration unknown) was obtained in analogous fashion from I-3c.

### Intermediate I-4b

### Preparation of Intermediate I-4b

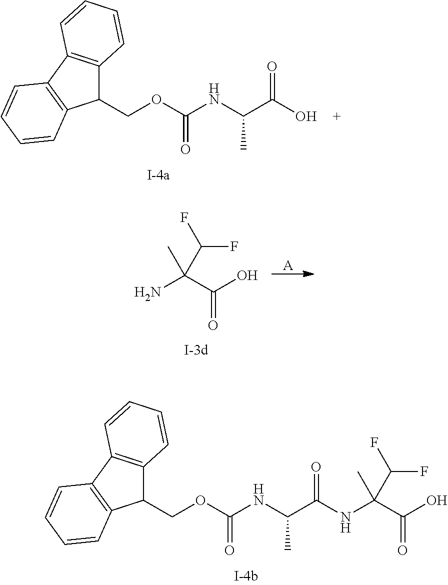

A mixture of (((9H-fluoren-9-yl)methoxy)carbonyl)-L-alanine (I-4a, 31 mg, 0.10 mmol), HATU (36 mg, 0.095 mmol) and Hunig's base (18 μL, 0.10 mmol) in DMF (0.50 mL) was stirred at room temperature for 2 min. At this point, 2-amino-3,3-difluoro-2-methylpropanoic acid (I-3d, 14 mg, 0.10 mmol) and additional Hunig's base (18 μL, 0.10 mmol) were added. The reaction mixture was stirred at room temperature for 1 hr, and then subjected to reverse phase column chromatography (20-60% MeCN/H₂O with 0.1% formic acid modifier) to afford 2-((S)-2-((((9H-fluoren-9-yl)methoxy)carbonyl)amino)propanamido)-3,3-difluoro-2-methylpropanoic acid (I-4b) as a solid. MS: m/z=455 [M+Na]. 1H NMR (500 MHz, DMSO-d₆) δ 13.02 (s, 1H), 8.47 (s, 1H), 7.89 (d, J=7.5 Hz, 2H), 7.73 (t, J=7.6 Hz, 2H), 7.53 (s, 1H), 7.42 (t, J=7.4 Hz, 2H), 7.33 (t, J=7.4 Hz, 2H), 6.29 (t, J=58.5 Hz, 1H), 4.31-4.17 (m, 3H), 4.15-4.04 (m, 1H), 1.37 (s, 3H), 1.21 (d, J=7.1 Hz, 3H).

### Intermediate I-5b

### Preparation of Intermediate I-5b

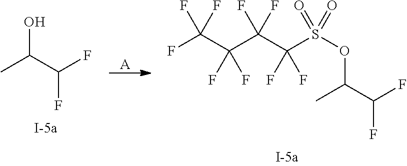

### Step A—Synthesis of Compound I-5b

At −78° C., a stirred solution of 1,1-difluoropropan-2-ol (I-5a, 0.21 mL, 2.5 mmol) in DCM (1.2 mL) was treated sequentially with triethylamine (1.1 mL, 7.5 mmol) and nonafluorobutanesulfonyl fluoride (0.76 mL, 4.3 mmol). The reaction mixture was allowed to gradually warm to room temperature and stirring was continued at room temperature overnight. The mixture was then poured into aqueous Na₂CO₃ and extracted with DCM twice. The combined organic layers were dried over MgSO4, filtered and concentrated under reduced pressure to afford 1,1-difluoropropan-2-yl 1,1,2,2,3,3,4,4,4-nonafluorobutane-1-sulfonate (I-5b), which was used crude directly.

### Example 2

### Preparation of Compound 4

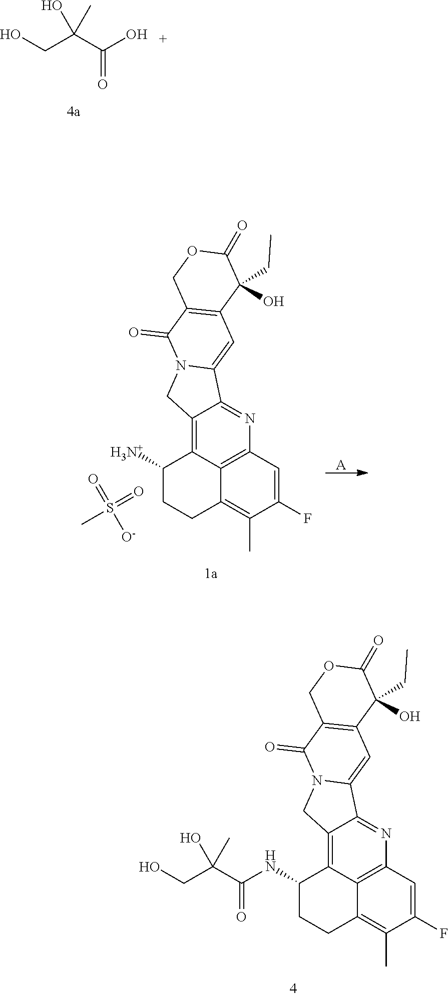

To a solution of 2,3-dihydroxy-2-methylpropanoic acid (4a, 11 mg, 0.090 mmol) in DMF (380 μl) was added (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 200 μl, 0.070-0.08 mmol, ˜0.35-0.40 M in DMF with 3.0 eq. Et₃N), HATU (34 mg, 0.090 mmol) and DIPEA (39 μl, 0.23 mmol). The resulting reaction mixture was stirred at room temperature for 1 hr. The mixture was then directly purified via reverse phase column chromatography (20-60% MeCN/water with 0.1% formic acid modifier) to afford N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,3-dihydroxy-2-methylpropanamide (4, peak 2) as a solid. MS: m/z=538 [M+H]. ¹H NMR (500 MHz, DMSO-d₆) δ 8.14 (d, J=8.8 Hz, 1H), 7.77 (d, J=10.9 Hz, 1H), 7.30 (s, 1H), 5.54-5.50 (m, 1H), 5.42 (s, 2H), 5.28 (d, J=19.1 Hz, 1H), 5.16 (d, J=19.1 Hz, 1H), 3.64 (d, J=10.7 Hz, 1H), 3.39 (d, J=10.7 Hz, 1H), 3.16 (br t, J=6.0 Hz, 2H), 2.39 (s, 3H), 2.25-2.19 (m, 1H), 2.15-2.08 (m, 1H), 1.90-1.81 (m, 2H), 1.24 (s, 3H), 0.87 (t, J=7.3 Hz, 3H).

The following compounds of the present disclosure in Table 2 were made using similar methods described in Example 2 with varying reaction times (10 min to 1 hr) and substituting the appropriate reactants and/or reagents:

TABLE 2

| | | MS |
|---|---|---|
| Compound | Structure | [M + H] |
| 5 (peak 1 from Example 2) | 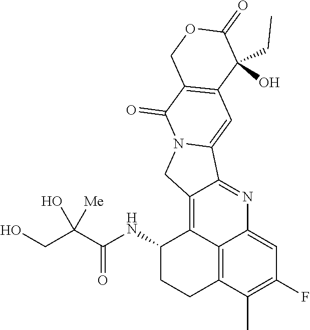 [SMILES: CC[C@@]1(O)C(=O)OCc2c1cc1n(c2=O)Cc2c-1nc1cc(F)c(C)c3c1c2[C@@H](NC(=O)C(C)(O)CO)CC3] | 538 |
| 6 | 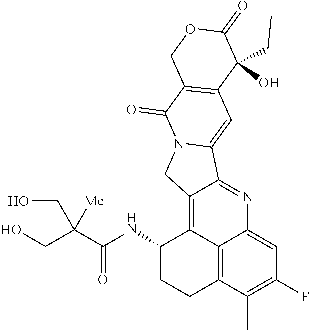 [SMILES: CC[C@@]1(O)C(=O)OCc2c1cc1n(c2=O)Cc2c-1nc1cc(F)c(C)c3c1c2[C@@H](NC(=O)C(C)(CO)CO)CC3] | 552 |
| 7 (peak 1) | 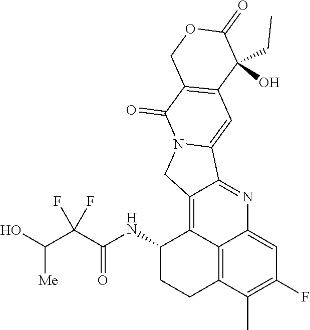 [SMILES: CC[C@@]1(O)C(=O)OCc2c1cc1n(c2=O)Cc2c-1nc1cc(F)c(C)c3c1c2[C@@H](NC(=O)C(F)(F)C(C)O)CC3] | 558 |
| 8 (peak 2) | 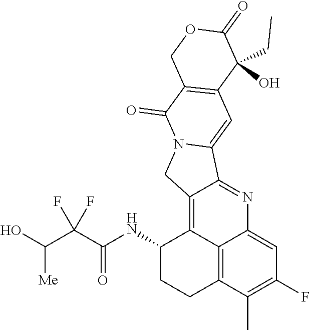 [SMILES: CC[C@@]1(O)C(=O)OCc2c1cc1n(c2=O)Cc2c-1nc1cc(F)c(C)c3c1c2[C@@H](NC(=O)C(F)(F)C(C)O)CC3] | 558 |
| 9 | 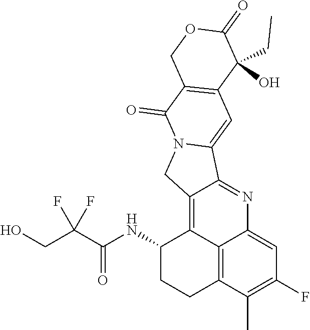 [SMILES: CC[C@@]1(O)C(=O)OCc2c1cc1n(c2=O)Cc2c-1nc1cc(F)c(C)c3c1c2[C@@H](NC(=O)C(F)(F)CO)CC3] | 544 |
| 10 (peak 1) | 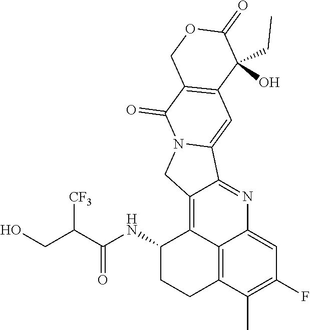 [SMILES: CC[C@@]1(O)C(=O)OCc2c1cc1n(c2=O)Cc2c-1nc1cc(F)c(C)c3c1c2[C@@H](NC(=O)C(CO)C(F)(F)F)CC3] | 576 |
| 11 (peak 2) | 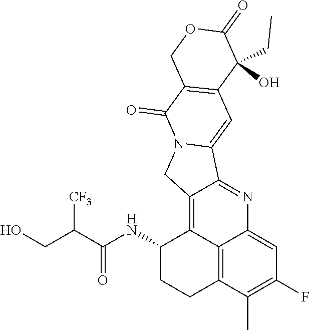 [SMILES: CC[C@@]1(O)C(=O)OCc2c1cc1n(c2=O)Cc2c-1nc1cc(F)c(C)c3c1c2[C@@H](NC(=O)C(CO)C(F)(F)F)CC3] | 576 |
| 12 | 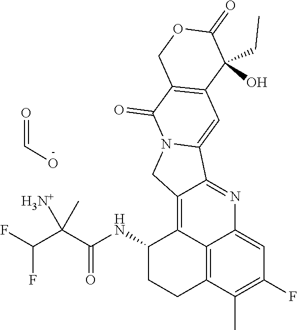 | 557 |
| | Made from I-3d | |
| 13 | 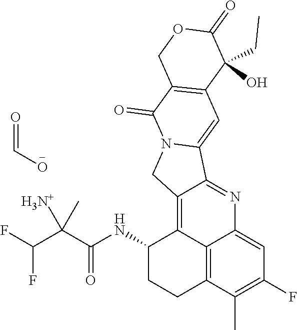 [SMILES: CC[C@@]1(O)C(=O)OCc2c1cc1n(c2=O)Cc2c-1nc1cc(F)c(C)c3c1c2[C@@H](NC(=O)C(C)([NH3+])C(F)F)CC3 ; O=CO] | 557 |
| | Made from I-3e | |
| 14 (peak 1) | 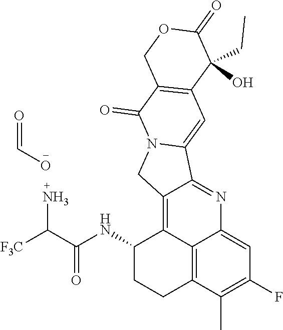 | 561 |
| 15 (peak 2) | 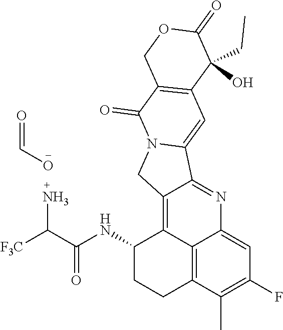 | 561 |
| 16 (peak 1) | 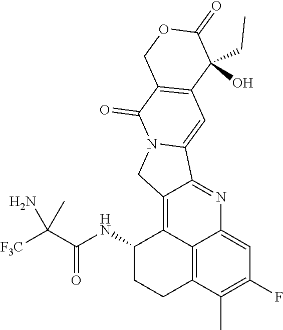 [SMILES: CC[C@@]1(O)C(=O)OCc2c1cc1n(c2=O)Cc2c-1nc1cc(F)c(C)c3c1c2[C@@H](NC(=O)C(C)(N)C(F)(F)F)CC3] | 575 |
| 17 (peak 2) | 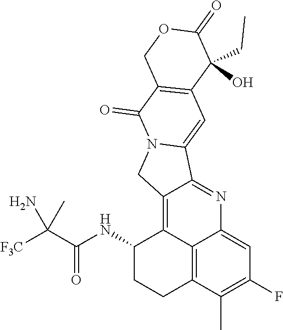 [SMILES: CC[C@@]1(O)C(=O)OCc2c1cc1n(c2=O)Cc2c-1nc1cc(F)c(C)c3c1c2[C@@H](NC(=O)C(C)(N)C(F)(F)F)CC3] | 575 |
| 18 (peak 1) | 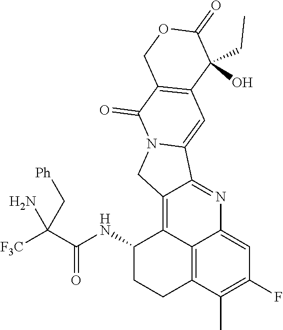 [SMILES: CC[C@@]1(O)C(=O)OCc2c1cc1n(c2=O)Cc2c-1nc1cc(F)c(C)c3c1c2[C@@H](NC(=O)C(N)(Cc1ccccc1)C(F)(F)F)CC3] | 651 |
| 19 (peak 2) | 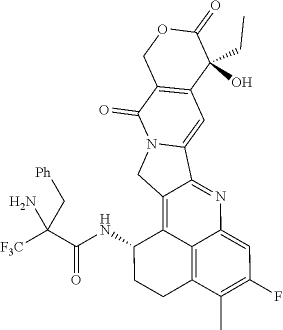 [SMILES: CC[C@@]1(O)C(=O)OCc2c1cc1n(c2=O)Cc2c-1nc1cc(F)c(C)c3c1c2[C@@H](NC(=O)C(N)(Cc1ccccc1)C(F)(F)F)CC3] | 651 |
| 20 | 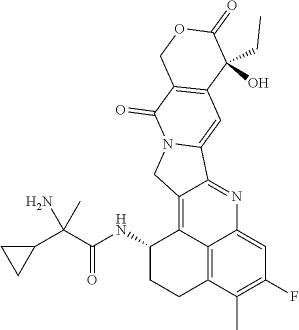 [SMILES: CC[C@@]1(O)C(=O)OCc2c1cc1n(c2=O)Cc2c-1nc1cc(F)c(C)c3c1c2[C@@H](NC(=O)C(C)(N)C1CC1)CC3] | 547 |
| | 1:1 mixture of diastereomers | |

### Example 3

### Preparation of Compound 21

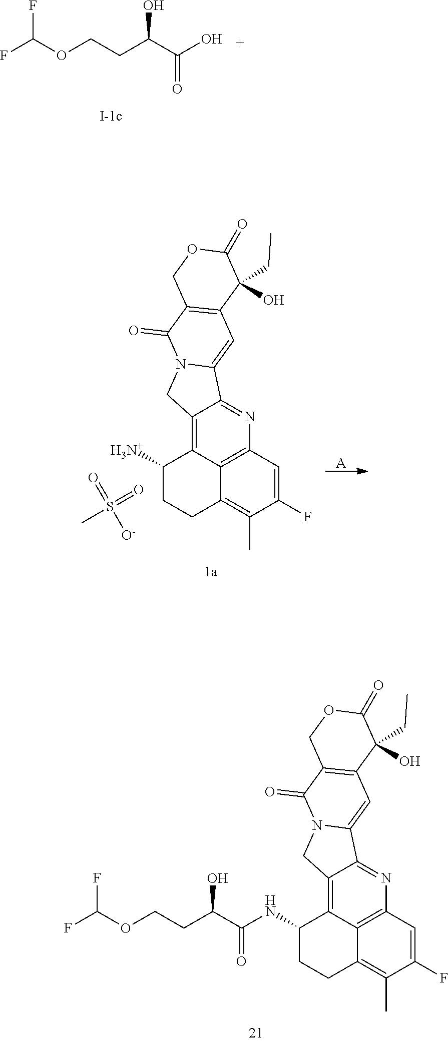

A solution of (R)-4-(difluoromethoxy)-2-hydroxybutanoic acid (I-1c, 18 mg, 0.10 mmol), HATU (39 mg, 0.10 mmol), and N-methylmorpholine (0.020 ml, 0.18 mmol) in DMF (1 mL) was stirred at RT for ˜10 min before (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 0.25 mL, 0.089-0.10 mmol, ˜0.35-0.40 M in DMF with 3.0 eq. Et3N) mixture was added. The reaction was stirred at RT for ˜1 hr 40 min. The material was filtered, and then subjected to reverse phase column chromatography (25-70% MeCN/water with 0.1% formic acid modifier) to yield (R)-4-(difluoromethoxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxybutanamide (21) as a solid. MS: m/z=588 [M+H]. ¹H NMR (500 MHz, DMSO-d₆) δ 8.53 (d, J=9.0 Hz, 1H), 7.79 (d, J=10.7 Hz, 1H), 7.31 (s, 1H), 6.68 (t, J=76.3 Hz, 1H, partly overlaps with doublet at 6.51 ppm), 6.51 (d, J=5.5 Hz, 1H, partly overlaps with triplet at 6.68 ppm), 5.77 (d, J=5.8 Hz, 1H), 5.61-5.54 (m, 1H), 5.42 (s, 2H), 5.25-5.12 (m, 2H), 4.11-4.05 (m, 1H), 4.01-3.94 (m, 2H), 2.40 (s, 3H), 2.23-2.11 (m, 3H), 2.03-1.92 (m, 2H), 1.92-1.81 (m, 2H), 0.87 (t, J=7.2 Hz, 3H).

The following compounds of the present disclosure in Table 3 were made using similar methods described in Example 3 with varying reaction times (up to 4 hrs) and substituting the appropriate reactants and/or reagents:

TABLE 3

| Com- | | MS |
|---|---|---|
| pound | Structure | [M + H] |
| 22 (peak 1) | 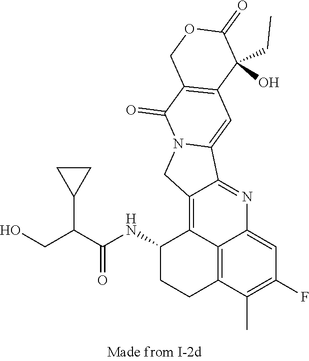 [SMILES: CC[C@@]1(O)C(=O)OCc2c1cc1n(c2=O)Cc2c-1nc1cc(F)c(C)c3c1c2[C@@H](NC(=O)C(CO)C1CC1)CC3] | 548 |
| 23 (peak 2) | 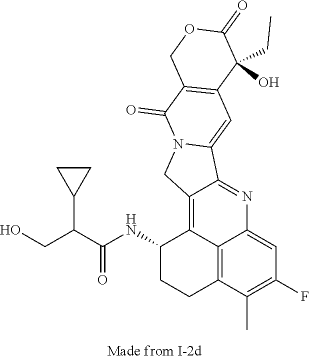 [SMILES: CC[C@@]1(O)C(=O)OCc2c1cc1n(c2=O)Cc2c-1nc1cc(F)c(C)c3c1c2[C@@H](NC(=O)C(CO)C1CC1)CC3] | 548 |

### Example 4

### Preparation of Compound 24

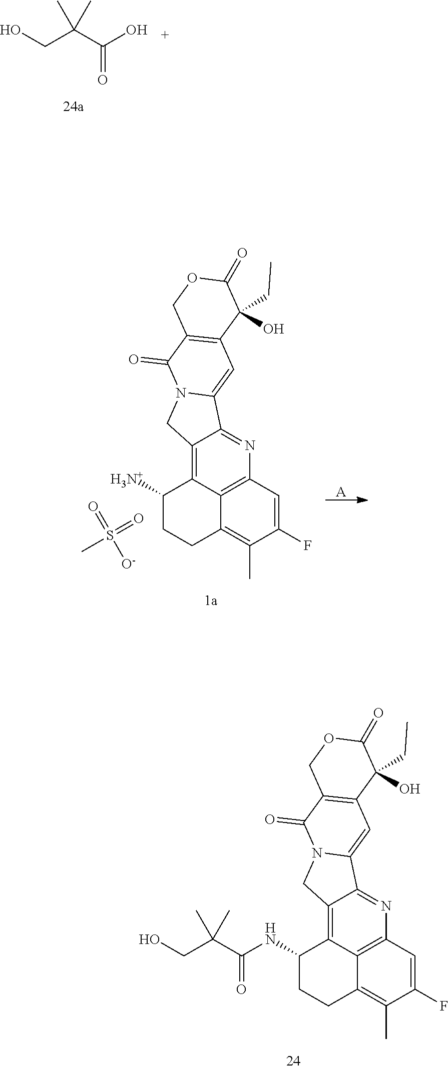

To a solution of 3-hydroxy-2,2-dimethylpropanoic acid (24a, 22 mg, 0.19 mmol) in DMF (380 μl) was added DIPEA (99 μl, 0.56 mmol) and (1S,9S)-9-ethyl-5-fluoro-9-hydroxy-4-methyl-10,13-dioxo-2,3,9,10,13,15-hexahydro-1H,12H-enzo[de]pyrano[3′,4′: 6,7]indolizino[1,2-b]quinolin-1-aminium methanesulfonate (1a, 250 μl, 0.089-0.10 mmol, ˜0.35-0.40 M in DMF with 3.0 eq. Et₃N), followed by addition of 1-propanephosphonic anhydride solution (in DMF) (150 μl, 0.25 mmol). The resulting mixture was stirred at room temperature for overnight, then purified via reverse phase column chromatography (5-60% MeCN/water with 0.10% formic acid modifier) to afford N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2,2-dimethylpropanamide (24) as a solid. MS: m/z=536 [M+H]. ¹H NMR (500 MHz, DMSO-d₆) δ 7.99 (d, J=8.5 Hz, 1H), 7.79 (d, J=11.0 Hz, 1H), 7.31 (s, 1H), 6.51 (s, 1H), 5.60-5.53 (m, 1H), 5.42 (s, 2H), 5.26-5.12 (m, 2H), 4.87 (t, J=5.1 Hz, 1H), 3.45 (dd, J=10.4, 5.0 Hz, 1H), 3.38 (dd, J=10.5, 4.9 Hz, 1H), 3.18-3.12 (m, 2H), 2.40 (s, 3H), 2.20-2.06 (m, 2H), 1.92-1.80 (m, 2H), 1.11 (d, J=9.8 Hz, 6H), 0.87 (t, J=7.3 Hz, 3H).

The following compounds of the present disclosure in Table 4 were made using similar methods described in Example 4 with varying reaction times (15 min to overnight) and substituting the appropriate reactants and/or reagents:

TABLE 4

| Com- | | MS |
|---|---|---|
| pound | Structure | [M + H] |
| 25 | 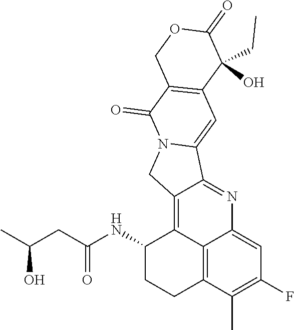 [SMILES: CC[C@@]1(O)C(=O)OCc2c1cc1n(c2=O)Cc2c-1nc1cc(F)c(C)c3c1c2[C@@H](NC(=O)C[C@H](C)O)CC3] | 522 |
| 26 | 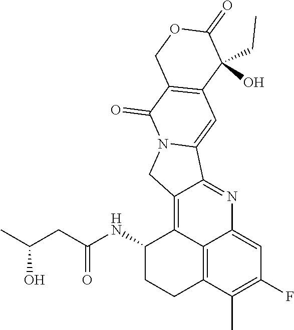 [SMILES: CC[C@@]1(O)C(=O)OCc2c1cc1n(c2=O)Cc2c-1nc1cc(F)c(C)c3c1c2[C@@H](NC(=O)C[C@@H](C)O)CC3] | 522 |
| 27 | 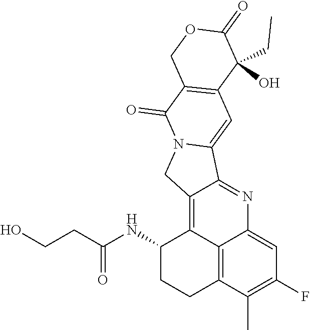 [SMILES: CC[C@@]1(O)C(=O)OCc2c1cc1n(c2=O)Cc2c-1nc1cc(F)c(C)c3c1c2[C@@H](NC(=O)CCO)CC3] | 508 |

### Example 5

### Preparation of Compound 28

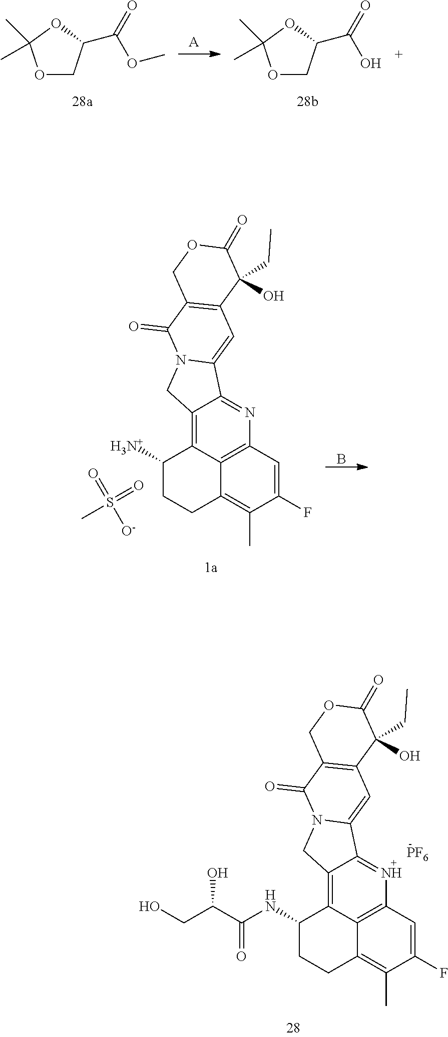

### Step A—Synthesis of Compound 28b

To a solution of methyl (S)-2,2-dimethyl-1,3-dioxolane-4-carboxylate (28a, 0.15 g, 0.94 mmol) in DCE (3.0 mL) was added trimethyltin hydroxide (0.25 g, 1.4 mmol). The reaction was stirred at 80° C. for 2.5 hr. The mixture was cooled, and diluted with EtOAc and dilute aqueous HCl (1.5 mmol). After shaking the mixture, the organic layer was isolated, passed through a hydrophobic membrane phase separator, and concentrated under reduced pressure to afford (S)-2,2-dimethyl-1,3-dioxolane-4-carboxylic acid (28b) as an oil, which was used crude directly in the next reaction.

### Step B—Synthesis of Compound 28

To a solution of (S)-2,2-dimethyl-1,3-dioxolane-4-carboxylic acid (28b, 22 mg, 0.15 mmol) in DMF (1.0 mL) was added Hunig's base (0.050 mL, 0.29 mmol), followed by HATU (57 mg, 0.15 mmol). The reaction was stirred at room temperature for 10 min before (1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-aminium methanesulfonate (1a, 0.30 mL, 0.11-0.12 mmol, ˜0.35-0.40 M in DMF with Hunig's base) mixture was added. The reaction was stirred at room temperature for 1 hr 40 min before another portion of (R)-2,2-dimethyl-1,3-dioxolane-4-carboxylic acid (30 mg, 0.21 mmol), HATU (70 mg, 0.18 mmol), and Hunig's base (0.050 mL, 0.29 mmol) in DMF (0.30 mL) was added. The reaction was stirred at room temperature for 20 min. Then, HCl (0.20 mL, 0.80 mmol, 4.0 M in dioxane) in water (0.20 mL) was added, and the reaction was stirred at room temperature overnight. Another portion of HCl (0.20 mL, 4.0 M in dioxane) was added, and 2 hr and 20 min later a final portion of HCl (0.10 mL, 4.0 M in dioxane) was added. The reaction was stirred at room temperature for another 4.5 hr. The mixture was then filtered and subjected to reverse phase column chromatography (25-60% MeCN/water with 0.1% formic acid modifier) to afford (1S,9S)-1-((S)-2,3-dihydroxypropanamido)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-7-ium hexafluorophosphate (V) (28, peak 1, PF₆ salt) as a solid. MS: m/z=524 [M+H]. ¹H NMR (500 MHz, DMSO-d₆) δ 8.34 (d, J=8.8 Hz, 1H), 7.79 (d, J=10.9 Hz, 1H), 7.31 (s, 1H), 6.52 (s, 1H), 5.57-5.51 (m, 1H), 5.47 (d, J=5.6 Hz, 1H), 5.42 (s, 2H), 5.22 (q, J=18.9 Hz, 2H), 4.72 (t, J=5.7 Hz, 1H), 4.03-3.99 (m, 1H), 3.66-3.60 (m, 1H), 3.57-3.51 (m, 1H), 3.23-3.10 (m, 2H), 2.40 (s, 3H), 2.27-2.19 (m, 1H), 2.12 (d, J=4.7 Hz, 1H), 1.92-1.81 (m, 2H), 0.87 (t, J=7.4 Hz, 3H).

The following compounds of the present disclosure in Table 5 were made using similar methods described in Example 5 substituting the appropriate reactants and/or reagents:

TABLE 5

| Com- | | MS |
|---|---|---|
| pound | Structure | [M + H] |
| 29 | 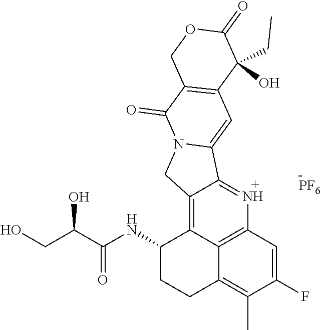 [SMILES: CC[C@@]1(O)C(=O)OCc2c1cc1n(c2=O)Cc2c-1nc1cc(F)c(C)c3c1c2[C@@H](NC(=O)[C@H](O)CO)CC3 ; "PF6"] | 524 |

### Example 6

### Preparation of Compound 30

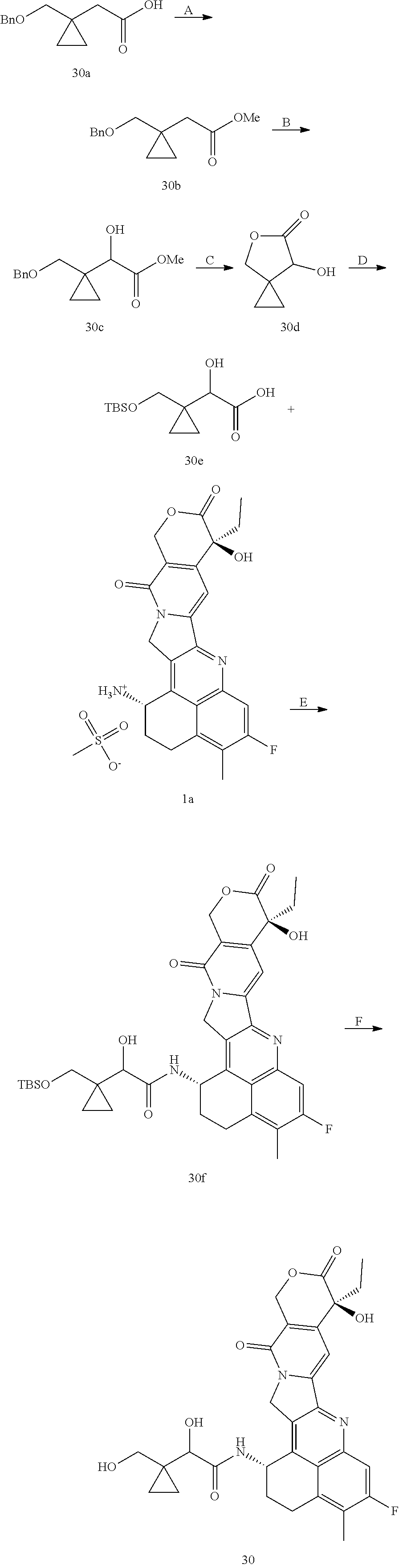

### Step A—Synthesis of Compound 30b

A mixture of 2-(1-((benzyloxy)methyl)cyclopropyl)acetic acid (30a, 3.0 g, 14 mmol), MeOH (23 mL) and sulfuric acid (16 mL, 0.31 mmol) was stirred at room temperature over 3 days. The solvent was removed under reduced pressure. The residue was treated with DCM and water, then neutralized to pH 7 with sodium bicarbonate. The two layers were separated and the water layer was washed with DCM twice more. The combined organic layers were dried over MgSO₄, filtered, and concentrated under reduced pressure to afford crude methyl 2-(1-((benzyloxy)methyl)cyclopropyl)acetate (30b) as an oil.

### Step B—Synthesis of Compound 30c

At −78° C., under argon, to a stirred solution of KHMDS (3.8 mL, 1.9 mmol, 0.5 M in toluene) was added dropwise a solution of methyl 2-(1-((benzyloxy)methyl)cyclopropyl)acetate (30b, 300 mg, 1.3 mmol) in THF (4.0 mL). The reaction mixture was stirred at −78° C. for 30 min, at which point a solution of 3-phenyl-2-(phenylsulfonyl)-1,2-oxaziridine (502 mg, 1.9 mmol) in THF (2.0 mL) was added dropwise. The reaction mixture was stirred at −78° C. for 30 min, then quenched with isopropanol and concentrated under reduced pressure. The residue was purified by silica gel column chromatography (0-50% EtOAc/hexanes) to afford methyl 2-(1-((benzyloxy)methyl)cyclopropyl)-2-hydroxy acetate (30c).

### Step C—Synthesis of Compound 30d

A mixture of 2-(1-((benzyloxy)methyl)cyclopropyl)-2-hydroxyacetate (30c, 100 mg, 0.40 mmol), 10 wt % Pd/C (21 mg, 0.020 mmol Pd) and MeOH (2.0 mL) was stirred under H₂ (1.0 atm) overnight. The reaction mixture was then filtered through Celite®, rinsing with MeOH. The solvent was removed under reduced pressure to afford crude 7-hydroxy-5-oxaspiro[2.4]heptan-6-one (30d). This crude mixture was used directly in the next step.

### Step D—Synthesis of Compound 30e

A mixture of crude 7-hydroxy-5-oxaspiro[2.4]heptan-6-one (30d, 51 mg, 0.40 mmol), lithium hydroxide (9.6 mg, 0.40 mmol) and anhydrous MeOH (1.6 mL) was stirred at room temperature overnight. The solvent was removed under reduced pressure. The residue was treated with DMF (1.6 mL), imidazole (123 mg, 1.8 mmol) and tert-butylchlorodimethylsilane (133 mg, 0.88 mmol). The reaction mixture was stirred at room temperature over 3 days. After removal of solvent under reduced pressure, the mixture was diluted with EtOAc and washed with aqueous 1M HCl. The aqueous layer was back-extracted with EtOAc twice more. The combined organic layers were dried over MgSO₄, filtered, and concentrated under reduced pressure to afford crude 2-(1-(((tert-butyldimethylsilyl)oxy)methyl)cyclopropyl)-2-hydroxyacetic acid (30e) as an oil, which was used directly in the next step.

### Step E—Synthesis of Compound 30f

To a stirred mixture of crude 2-(1-(((tert-butyldimethylsilyl)oxy)methyl)cyclopropyl)-2-hydroxyacetic acid (30e, 84 mg, 0.32 mmol), Hunig's base (73 μL, 0.42 mmol), (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 380 μl, 0.13-0.15 mmol, ˜0.35-0.40 M in DMF with 3.0 eq Et₃N) and DMF (0.30 mL) was added HATU (75 mg, 0.20 mmol). The reaction mixture was stirred at room temperature for 20 min, filtered through a syringe filter, and then subjected to reverse phase column chromatography (10-95% MeCN/H₂O with 0.1% formic acid modifier) to afford 2-(1-(((tert-butyldimethylsilyl)oxy)methyl)cyclopropyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxyacetamide (30f).

### Step F—Synthesis of Compound 30

A mixture of crude 2-(1-(((tert-butyldimethylsilyl)oxy)methyl)cyclopropyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxyacetamide (30f, 10 mg, 0.015 mmol), TFA (18 μL, 0.23 mmol), DMF (300 μL, 0.42 mmol) and MeOH (100 μL) was stirred at room temperature for 5 hr. The resulting suspension was then solubilized with DMSO and subjected to reverse phase column chromatography (10-100% MeCN/H₂O with 0.1% formic acid modifier) to afford N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxy-2-(1-(hydroxymethyl)cyclopropyl)acetamide (30, 1:1 mix of diastereomers) as a solid. MS: m/z=564 [M+H]. 1H NMR (500 MHz, DMSO-d₆) δ 8.62 (d, J=8.4 Hz, 1H), 8.30 (d, J=8.8 Hz, 1H), 7.79 (apparent t, J=11.7 Hz, 2H), 7.31 (s, 2H), 6.52 (s, 2H), 5.64-5.51 (m, 2H), 5.42 (s, 4H), 5.31-5.07 (m, 4H), 4.26 (d, J=11.0 Hz, 1H), 3.90 (s, 1H), 3.77 (s, 1H), 3.44 (s, 2H, overlapping with water peak), 3.23-3.11 (m, 5H), 2.41 (s, 3H), 2.40 (s, 3H), 2.27-2.05 (m, 4H), 1.93-1.78 (m, 4H), 0.92-0.83 (m, 6H), 0.78-0.72 (m, 1H), 0.70-0.60 (m, 3H), 0.55-0.46 (m, 2H), 0.45-0.37 (m, 2H).

### Example 7

### Preparation of Compound 31

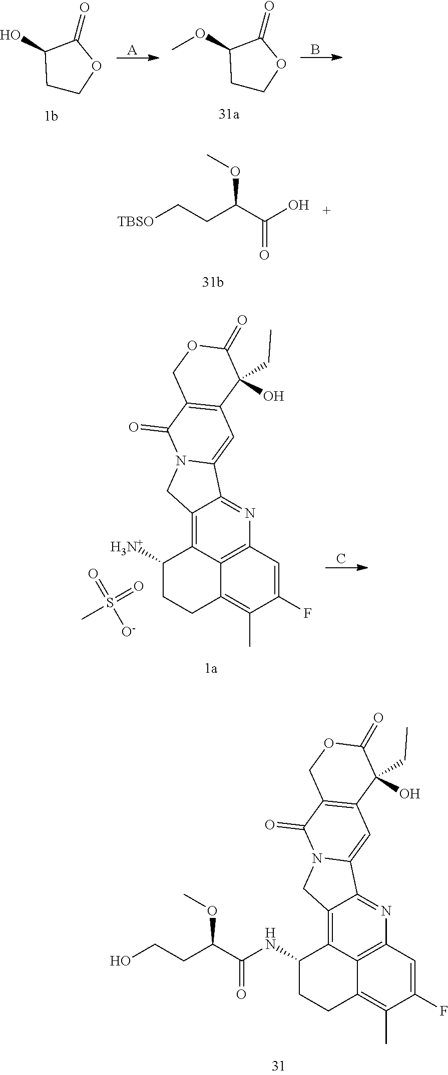

### Step A—Synthesis of Compound 31a

A mixture of (R)-3-hydroxydihydrofuran-2(3H)-one (1b, 600 mg, 5.9 mmol), methyl iodide (1.8 mL, 29 mmol), silver oxide (1.5 g, 6.6 mmol) and acetonitrile (12 mL) was stirred under argon, in the dark, at 75° C. overnight. The mixture was then filtered through Celite®, rinsing with acetonitrile, and the solvent was removed under reduced pressure. The residue was redissolved in DCM, re-filtered, and concentrated under reduced pressure to afford crude (R)-3-methoxydihydrofuran-2(3H)-one (31a) as an oil.

### Step B—Synthesis of Compound 3b

A mixture of (R)-3-methoxydihydrofuran-2(3H)-one (31a, 350 mg, 3.0 mmol), lithium hydroxide (76 mg, 3.2 mmol) and methanol (6.0 mL) was stirred at room temperature over 3 days. The solvent was then thoroughly removed under reduced pressure. To the resulting residue were added DMF (6.0 mL), imidazole (620 mg, 9.0 mmol) and tert-butylchlorodimethylsilane (590 mg, 3.9 mmol). The reaction mixture was stirred at room temperature overnight, at which point most of the solvent was removed under reduced pressure. The residue was diluted with DCM and washed with 1M aqueous HCl. The aqueous layer was back-extracted with DCM twice more. The combined organic layers were dried over MgSO₄, filtered, and concentrated under reduced pressure to afford crude (R)-4-((tert-butyldimethylsilyl)oxy)-2-methoxybutanoic acid (31b) as an oil, which was used directly in the next step.

### Step C—Synthesis of Compound 31

To a stirred mixture of (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 290 μL, 0.10-0.12 mmol, ˜0.35-0.40 M in DMF with 3.0 eq Et₃N), crude (R)-4-((tert-butyldimethylsilyl)oxy)-2-methoxybutanoic acid (31b, 140 mg, 0.57 mmol), Hunig's base (35 μL, 0.20 mmol) and DMF (0.10 mL) was added HATU (65 mg, 0.17 mmol). The mixture was stirred at room temperature for 10 min, at which point MeOH (0.10 mL) and TFA (77 μL, 1.0 mmol) were added. The mixture was stirred at room temperature for 2 hr. The resulting suspension was then diluted with DMSO (0.30 mL) and subjected to reverse phase column chromatography (10-70% MeCN/H₂O with 0.1% formic acid modifier) to afford (R)—N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methoxybutanamide (31) as a solid. MS: m/z=552 [M+H]. ¹H NMR (500 MHz, DMSO-d₆) δ 8.62 (d, J=8.9 Hz, 1H), 7.75 (d, J=10.9 Hz, 1H), 7.29 (s, 1H), 6.51 (s, 1H), 5.64-5.54 (m, 1H), 5.41 (s, 2H), 5.12 (s, 2H), 4.54 (s, 1H), 3.82 (dd, J=8.5, 4.4 Hz, 1H), 3.51 (t, J=6.3 Hz, 2H), 3.31 (s, 3H), 3.24-3.09 (m, 2H), 2.37 (s, 3H), 2.21-2.09 (m, 2H), 1.94-1.75 (m, 4H), 0.87 (t, J=7.3 Hz, 3H).

### Example 8

### Preparation of Compound 32

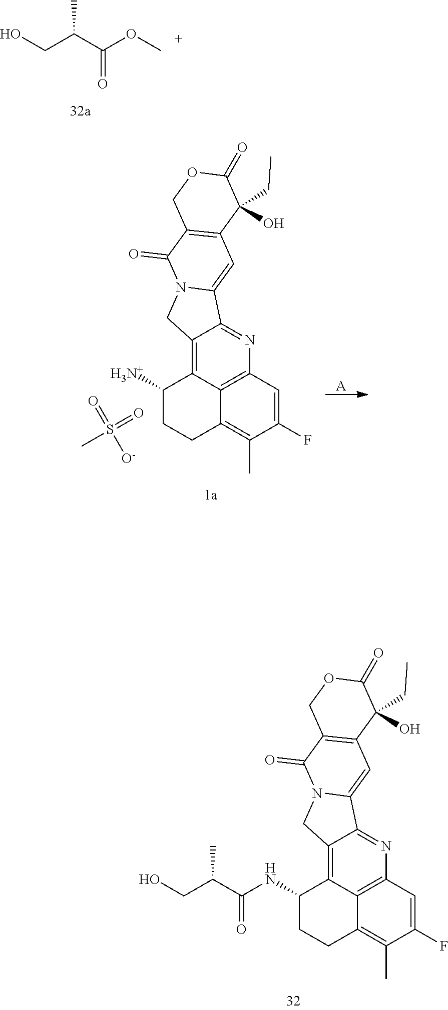

A mixture of methyl (S)-3-hydroxy-2-methylpropanoate (32a, 17 mg, 0.14 mmol) and lithium hydroxide (3.4 mg, 0.14 mmol) was dissolved in DMF (190 μl) and stirred at room temperature overnight. Then, (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 270 μl, 0.095-0.11 mmol, ˜0.35-0.40 M in DMF with 3.0 eq. Et3N) was added, followed by addition of HATU (43 mg, 0.11 mmol) and DIPEA (33 μl, 0.19 mmol). The resulting mixture was stirred at room temperature for 20 min then purified via reverse phase column chromatography (20-60% MeCN/water with 0.1% formic acid modifier) to afford (S)—N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-methylpropanamide (32) as a solid. MS: m/z=522 [M+H]. 1H NMR (500 MHz, DMSO-d₆) δ 8.38 (d, J=8.7 Hz, 1H), 7.77 (d, J=11.0 Hz, 1H), 7.29 (s, 1H), 6.52 (s, 1H), 5.60-5.53 (m, 1H), 5.42 (s, 2H), 5.21 (s, 2H), 4.62 (s, 1H), 3.61-3.51 (m, 1H), 3.36-3.34 (m, 1H), 3.22-3.10 (m, 2H), 2.47-2.40 (m, 1H), 2.38 (s, 3H), 2.20-2.05 (m, 2H), 1.94-1.79 (m, 2H), 1.01 (d, J=6.9 Hz, 3H), 0.87 (t, J=7.3 Hz, 3H).

The following compounds of the present disclosure in Table 6 were made using similar methods described in Example 8 with varying reaction times (2 hr for LiOH phase, 15-30 min for coupling phase) and substituting the appropriate reactants and/or reagents:

TABLE 6

| Com- | | MS |
|---|---|---|
| pound | Structure | [M + H] |
| 33 | 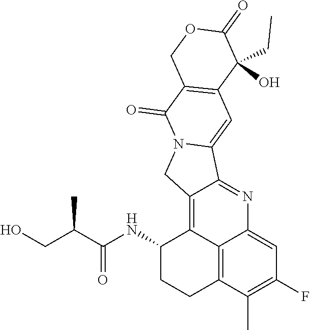 [SMILES: CC[C@@]1(O)C(=O)OCc2c1cc1n(c2=O)Cc2c-1nc1cc(F)c(C)c3c1c2[C@@H](NC(=O)[C@H](C)CO)CC3] | 522 |
| 34 (peak 1) | 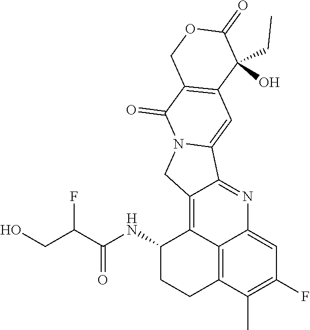 [SMILES: CC[C@@]1(O)C(=O)OCc2c1cc1n(c2=O)Cc2c-1nc1cc(F)c(C)c3c1c2[C@@H](NC(=O)C(F)CO)CC3] | 526 |
| 35 (peak 2) | 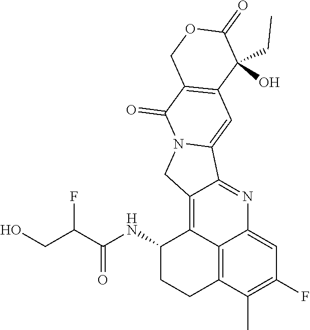 [SMILES: CC[C@@]1(O)C(=O)OCc2c1cc1n(c2=O)Cc2c-1nc1cc(F)c(C)c3c1c2[C@@H](NC(=O)C(F)CO)CC3] | 526 |
| 36 (peak 1) | 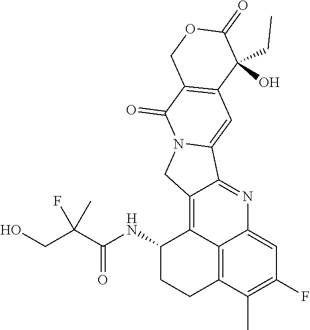 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] | 540 |
| 37 (peak 2) | 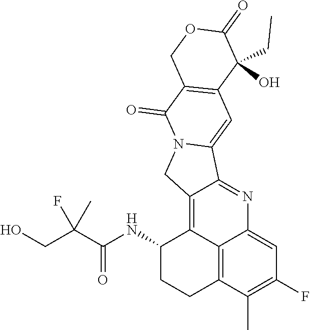 [SMILES: CC[C@@]1(O)C(=O)OCc2c1cc1n(c2=O)Cc2c-1nc1cc(F)c(C)c3c1c2[C@@H](NC(=O)C(C)(F)CO)CC3] | 540 |

### Example 9

### Preparation of Compound 38

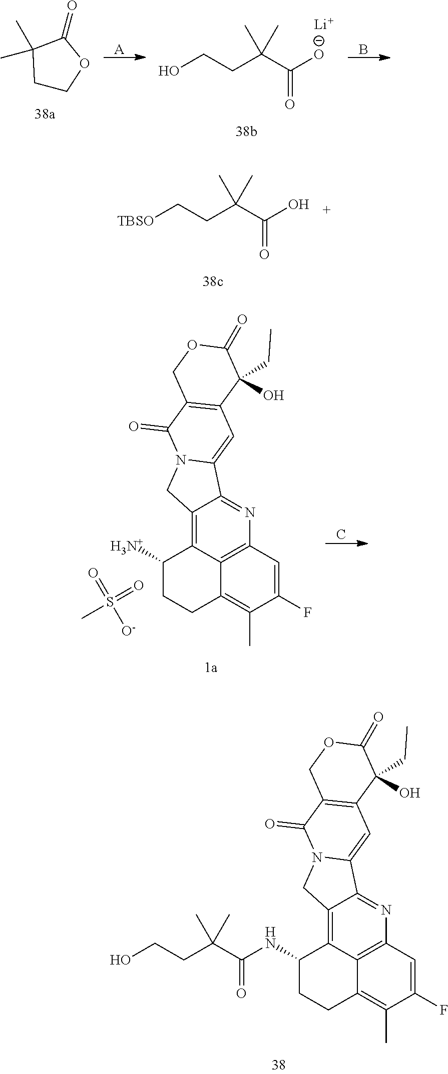

### Step A—Synthesis of Compound 38b

To a mixture of 3,3-dimethyldihydrofuran-2(3H)-one (38a, 0.068 g, 0.60 mmol) and lithium hydroxide (0.014 g, 0.60 mmol) was added ethanol (2.0 mL). The reaction was heated to 60° C. overnight. The reaction was cooled and concentrated under reduced pressure. The resulting crude lithium 4-hydroxy-2,2-dimethylbutanoate (38b) as a solid was used directly in the next reaction.

### Step B—Synthesis of Compound 38c

To a mixture of lithium 4-hydroxy-2,2-dimethylbutanoate (38b, 0.079 g, 0.60 mmol), DMAP (7.0 mg, 0.060 mmol), and TBSCl (0.10 g, 0.66 mmol) in DMF (3.0 mL) was added TEA (0.10 mL, 0.72 mmol). The reaction was stirred at room temperature overnight. The mixture was diluted with DCM, water, and 1M HCl (1.5 mL). The mixture was extracted and passed through a hydrophobic membrane phase separator. The organic layer was concentrated under reduced pressure, and the resulting crude 4-((tert-butyldimethylsilyl)oxy)-2,2-dimethylbutanoic acid (38c) was used directly in the next reaction.

### Step C—Synthesis of Compound 38

To a solution of 4-((tert-butyldimethylsilyl)oxy)-2,2-dimethylbutanoic acid (38c, 150 mg, 0.60 mmol) and N-methylmorpholine (0.050 mL, 0.46 mmol) in DMF (1.0 mL) was added HATU (170 mg, 0.45 mmol). The reaction was stirred at room temperature for 5 min before (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 0.30 mL, 0.11-0.12 mmol, ˜0.35-0.40 M in DMF with 3.0 eq. Et₃N) mixture was added. The reaction was stirred at room temperature for 2 hr 40 min. Then, MeOH (0.15 mL) was added, followed by TFA (0.20 mL). The reaction was stirred at room temperature for 1 hr. Then, TEA (0.30 mL) was added, and the mixture was filtered and subjected to reverse phase column chromatography (25-65% MeCN/water with 0.1% formic acid modifier) to afford N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2,2-dimethylbutanamide (38) as a solid. MS: m/z=550 [M+H]. ¹H NMR (500 MHz, DMSO-d₆): δ 8.07 (d, J=8.4 Hz, 1H), 7.79 (d, J=10.9 Hz, 1H), 7.31 (s, 1H), 5.60-5.54 (m, 1H), 5.42 (s, 2H), 5.22-5.08 (m, 2H), 3.46-3.41 (m, 4H) (signal overlapping with broad water peak), 3.20-3.11 (m, 3H) (signal overlapping with broad water peak), 2.40 (s, 3H), 2.16-2.06 (m, 2H), 1.90-1.81 (m, 2H), 1.72 (t, J=7.4 Hz, 2H), 1.17 (d, J=6.6 Hz, 6H), 0.88 (t, J=7.3 Hz, 3H).

### Example 10

### Preparation of Compounds 39 and 40

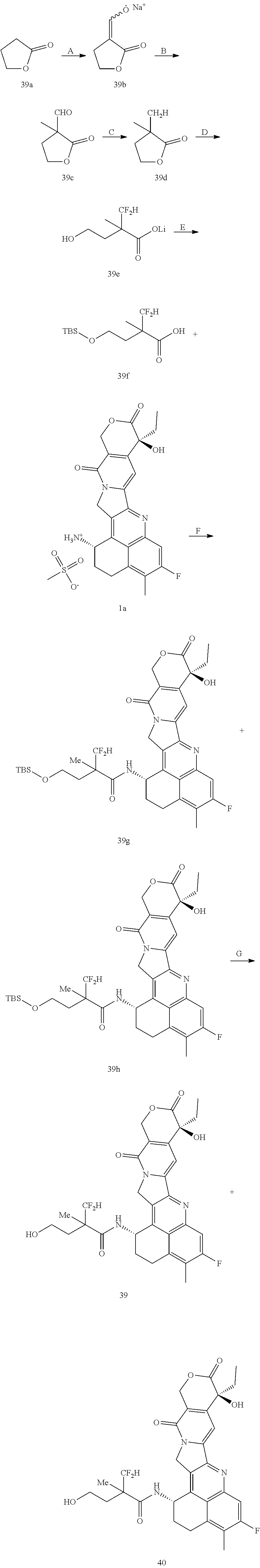

### Step A—Synthesis of Compound 39b

Dihydrofuran-2(3H)-one (39a, 10 g, 116 mmol) and ethyl formate (8.6 g, 120 mmol) were added dropwise to a stirred mixture of NaH (4.7 g, 120 mmol, 60 wt %) in hexane (100 mL) at room temperature. After the addition of about 10% of the mixture, a small amount of absolute ethanol (0.70 mL) was added to initialize the reaction, then the mixture was refluxed at 70° C. for 2 hr. The mixture was filtered and the filter cake was washed with hexane (60 mL), and dried under vacuum to give crude sodium (Z)-(2-oxodihydrofuran-3(2H)-ylidene)methanolate (39b) as a solid, which was used directly in the next reaction.

### Step B—Synthesis of Compound 39c

Sodium (Z)-(2-oxodihydrofuran-3(2H)-ylidene)methanolate (39b, 15 g, 110 mmol) (freshly made) in CH₃I (43 mL) in a round bottomed flask with reflux condenser was heated to reflux (60° C.) under an atmosphere of nitrogen for 48 hr. The reaction mixture was cooled to room temperature and filtered. The flask and the filter residue were washed with chloroform (CHCl₃), and the combined filtrates were evaporated under reduced pressure. The filtrate was purified by silica gel column chromatography (1:1 petroleum ether:EtOAc) to give 3-methyl-2-oxotetrahydrofuran-3-carbaldehyde (39c) as an oil.

### Step C—Synthesis of Compound 39d

DAST (19 mL, 140 mmol) was added dropwise to a solution of 3-methyl-2-oxotetrahydrofuran-3-carbaldehyde (39c, 6.0 g, 47 mmol) in DCM (60 mL) at 0° C. The reaction mixture was stirred for 18 hr at room temperature. The mixture was added to saturated aqueous NaHCO₃ (300 mL), then adjusted to pH 8, and the mixture was extracted with DCM (3×60 mL). The combined organic fractions were washed with brine (20 mL), dried over Na₂SO₄, filtered, and the solvent was evaporated under reduced pressure to give crude 3-(difluoromethyl)-3-methyldihydrofuran-2(3H)-one (39d) as an oil. The material was used directly in the next step.

### Step D—Synthesis of Compound 39e

LiOH (0.96 g, 40 mmol) was added to a stirred mixture of 3-(difluoromethyl)-3-methyldihydrofuran-2(3H)-one (39d, 2.0 g, 13 mmol) in MeOH/THF/water (1:1:1) (10 mL) at room temperature, and the mixture was stirred at room temperature for 18 hr. The solvent was evaporated under reduced pressure to give crude lithium 2-(difluoromethyl)-4-hydroxy-2-methylbutanoate (39e) as a solid, which was used directly in the next step.

### Step E—Synthesis of Compound 39f

TBSCl (0.43 g, 2.9 mmol) was added to a stirred mixture of imidazole (0.32 g, 4.8 mmol) and lithium 2-(difluoromethyl)-4-hydroxy-2-methylbutanoate (39e, 0.40 g, 2.4 mmol) in DMF (6.0 mL) at room temperature, and the mixture was stirred at room temperature for 18 hr. The material was purified by silica gel column chromatography (10% EtOAc/petroleum ether) to give 4-((tert-butyldimethylsilyl)oxy)-2-(difluoromethyl)-2-methylbutanoic acid (39f) as an oil, which was used directly in the next step.

### Step F—Synthesis of Compounds 39g and 39h

DIPEA (0.10 ml, 0.57 mmol) and HATU (86 mg, 0.23 mmol) were added to a stirred mixture of 4-((tert-butyldimethylsilyl)oxy)-2-(difluoromethyl)-2-methylbutanoic acid (39f, 500 mg, 1.8 mmol) in DMF (2.0 mL) at room temperature and stirred for 5 min before (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 100 mg, 0.19 mmol) was added. The mixture was stirred at room temperature for 30 min. The material was purified by reverse phase column chromatography (75-100% MeCN/water with 0.1% formic acid modifier) to give 4-((tert-butyldimethylsilyl)oxy)-2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)butanamide (39g, peak 1) as a solid and 4-((tert-butyldimethylsilyl)oxy)-2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)butanamide (39h, peak 2) as a solid.

MS: m/z=700 [M+H].

### Step G—Synthesis of Compounds 39 and 40

TFA (0.050 ml, 0.65 mmol) was added to a stirred mixture of 4-((tert-butyldimethylsilyl)oxy)-2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-methylbutanamide (40 mg, 0.057 mmol, 39g, peak 1) in DMF/MeOH (0.75 mL) at room temperature, and the mixture was stirred at room temperature for 1 hr. Then, more TFA (0.10 mL) was added, and the mixture was stirred at room temperature for 0.5 hr. The residue was purified by reverse phase column chromatography (30-60% MeCN/water with 0.1% formic acid modifier) followed by reverse phase column chromatography again (26-56% MeCN/water with 0.1% formic acid modifier) to give 2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methylbutanamide (40) as a solid. MS: m/z=586 [M+H]. 1H NMR (DMSO-d₆, 400 MHz) δ 8.36 (br d, 1H, J=8.1 Hz), 7.81 (d, 1H, J=11.0 Hz), 7.32 (s, 1H), 6.52 (s, 1H), 6.1-6.5 (m, 1H), 5.6-5.7 (m, 1H), 5.42 (s, 2H), 5.2-5.3 (m, 1H), 5.05 (br d, 1H, J=18.8 Hz), 4.65 (t, 1H, J=4.8 Hz), 3.40-3.70 (m, 2H), 3.11-3.20 (m, 2H), 2.41 (s, 3H), 2.01-2.21 (m, 2H), 1.84-1.88 (m, 3H), 1.60-1.71 (m, 1H), 1.22 (s, 3H), 0.88 (br t, 3H, J=7.3 Hz).

TFA (0.050 ml, 0.65 mmol) was added to a stirred mixture of 4-((tert-butyldimethylsilyl)oxy)-2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-methylbutanamide (39h, 45 mg, 0.064 mmol, peak 2) in DMF/MeOH (0.75 mL) at room temperature, and the mixture was stirred at room temperature for 1 hr. The residue was purified by reverse phase column chromatography (30-60% MeCN/water with 0.1% formic acid modifier) to give 2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methylbutanamide (39) as a solid. MS: m/z=586 [M+H]. 1H NMR (DMSO-d₆, 400 MHz) δ 8.42 (d, 1H, J=8.1 Hz), 7.86 (d, 1H, J=11.0 Hz), 7.36 (s, 1H), 6.59 (s, 1H), 6.27 (br t, 1H, J=56.3 Hz), 5.6-5.8 (m, 1H), 5.47 (s, 2H), 5.3-5.4 (m, 1H), 5.07 (d, 1H, J=18.8 Hz), 4.70 (t, 1H, J=4.9 Hz), 3.5-3.6 (m, 2H), 3.1-3.3 (m, 2H), 2.45 (s, 3H), 2.07-2.17 (m, 2H), 1.84-1.93 (m, 3H), 1.61-1.67 (m, 1H), 1.27 (s, 3H), 0.87 (t, 3H, J=7.2 Hz).

### Example 11

### Preparation of Compound 41

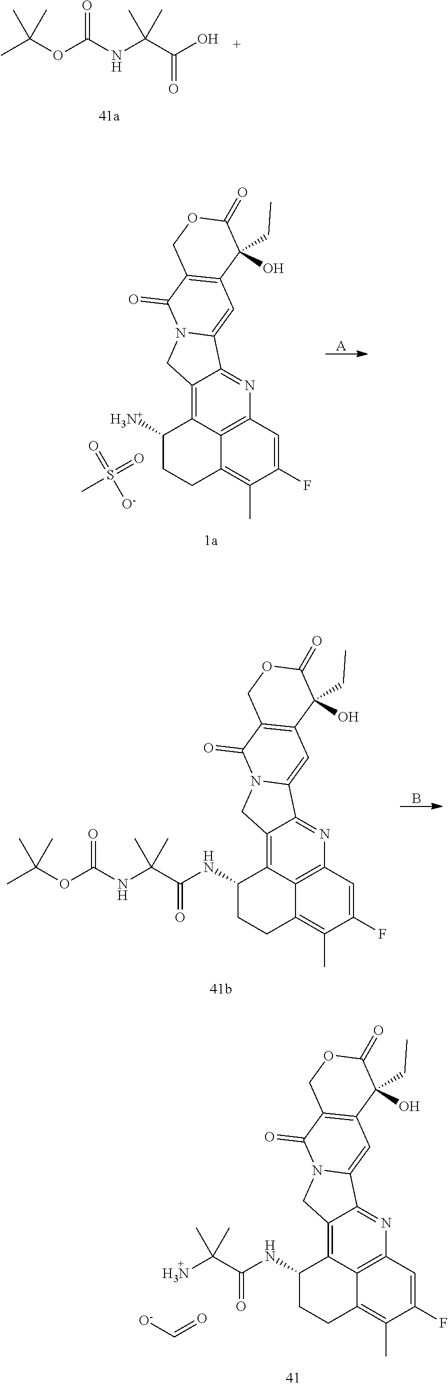

### Step A—Synthesis of Compound 41b

To a stirred mixture of 2-((tert-butoxycarbonyl)amino)-2-methylpropanoic acid (41a, 31 mg, 0.15 mmol), Hunig's Base (35 μL, 0.20 mmol), (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 290 μL, 0.10-0.12 mmol, ˜0.35-0.40 M in DMF with 3 eq. Et₃N) and DMF (0.20 mL) was added HATU (57 mg, 0.15 mmol). The reaction mixture was stirred at room temperature for 10 min, filtered through a syringe filter, and then subjected to reverse phase column chromatography (20-100% MeCN/H₂O with 0.1% formic acid modifier) to afford tert-butyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-methyl-1-oxopropan-2-yl)carbamate (41b) as a solid. MS: m/z=621 [M+H].

### Step B—Synthesis of Compound 41

A mixture of tert-butyl (1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-methyl-1-oxopropan-2-yl)carbamate (41b, 58 mg, 0.094 mmol), 4M HCl in dioxane (0.35 mL, 1.4 mmol) and dioxane (0.65 mL) was stirred at room temperature for 2.5 hr and monitored by LCMS. The mixture was then diluted with H₂O and subjected to reverse phase column chromatography (10-70% MeCN/H₂O with 0.1% formic acid modifier) to afford 2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-methylpropanamide formate (41) as a solid. MS: m/z=521 [M+H]. ¹H NMR (500 MHz, DMSO-d₆) δ 8.55 (s, 1H), 8.27 (s, 1H), 7.78 (d, J=10.9 Hz, 1H), 7.30 (s, 1H), 5.53 (t, J=5.2 Hz, 1H), 5.41 (s, 2H), 5.16 (s, 2H), 3.16 (t, J=6.1 Hz, 2H), 2.39 (s, 3H), 2.22-2.08 (m, 2H), 1.93-1.78 (m, 2H), 1.37 (s, 3H), 1.33 (s, 3H), 0.87 (t, J=7.3 Hz, 3H).

The following compounds of the present disclosure in Table 7 were made using similar methods described in Example 11 with varying reaction times (10-30 min for Step A, 3-7 hr for Step B) and substituting the appropriate reactants and/or reagents:

TABLE 7

| Com- | | MS |
|---|---|---|
| pound | Structure | [M + H] |
| 42 | 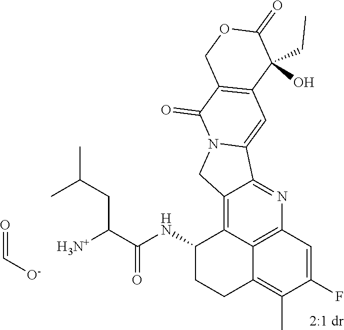 | 549 |
| 43 | 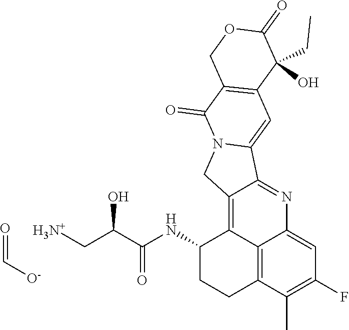 | 523 |
| 44 | 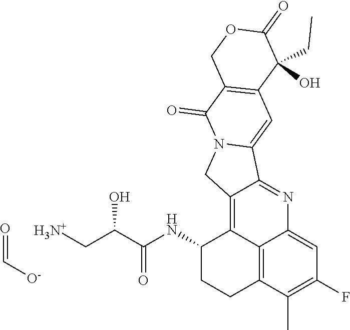 | 523 |

### Example 12

### Preparation of Compound 45

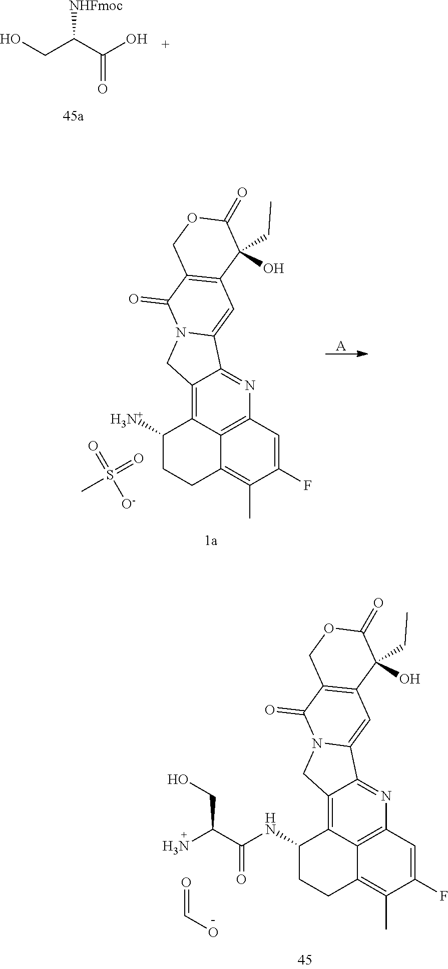

### Step A—Synthesis of Compound 45

To a stirred mixture of (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 270 μL, 0.095-0.11 mmol, ˜0.35-0.40 M in DMF with 3 eq. Et₃N), (((9H-fluoren-9-yl)methoxy)carbonyl)-L-serine (45a, 39 mg, 0.12 mmol), Hunig's base (18 μL, 0.10 mmol) and DMF (270 μL) was added HATU (46 mg, 0.12 mmol). The reaction mixture was stirred at room temperature for 5 min, at which point 4-methylpiperidine (59 μL, 0.50 mmol) was added. The mixture was further stirred at room temperature for 45 min, monitoring by LCMS. Afterwards, the reaction mixture was subjected to reverse phase column chromatography (10-60% MeCN/H₂O with 0.1% formic acid modifier) to afford (S)-2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide formate (45) as a solid. MS: m/z=523 [M+H]. ¹H NMR (500 MHz, DMSO-d₆) δ 8.70 (s, 1H), 8.23 (s, 1H), 7.79 (d, J=10.9 Hz, 1H), 7.30 (s, 1H), 5.57 (apparent s, 1H), 5.42 (s, 2H), 5.27-5.14 (m, 2H), 3.63-3.44 (m, 3H), 3.24-3.08 (m, 2H), 2.39 (s, 3H), 2.26-2.18 (m, 1H), 2.17-2.05 (m, 1H), 1.97-1.76 (m, 2H), 0.87 (t, J=7.3 Hz, 3H).

The following compounds of the present disclosure in Table 8 were made using similar methods described in Example 12 with varying reaction times (5 min to 2 hr for coupling phase, 30 min to 14 hr and up to 40° C. for Fmoc deprotection phase) and substituting the appropriate reactants and/or reagents:

TABLE 8

| Com- | | MS |
|---|---|---|
| pound | Structure | [M + H] |
| 46 | 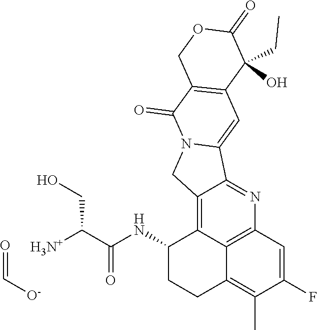 | 523 |
| 47 | 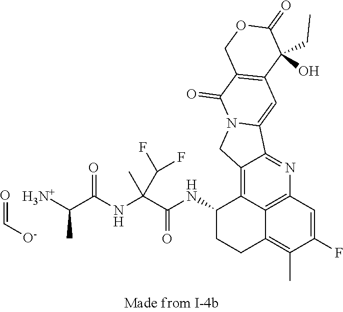 | 628 |
| 48 | 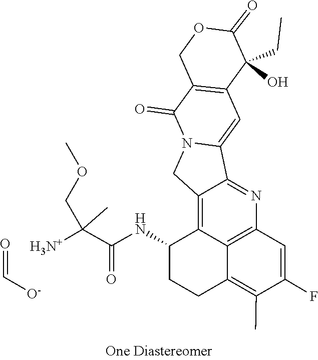 | 551 |

### Example 13

### Preparation of Compound 49

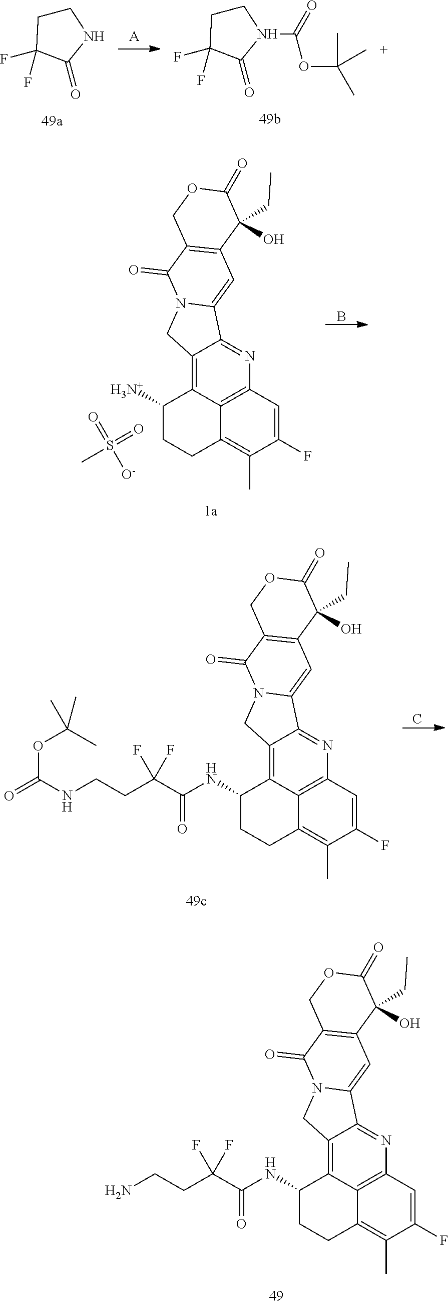

### Step A—Synthesis of Compound 49b

To a mixture of 3,3-difluoropyrrolidin-2-one (49a, 1.6 g, 13 mmol), Boc-anhydride (6.1 mL, 26 mmol) and DMF (13 mL) was added DMAP (0.16 g, 1.3 mmol). The reaction mixture was stirred at room temperature overnight. The mixture was then diluted with DCM, washed with brine three times, dried over MgSO₄, filtered and concentrated under reduced pressure to afford crude tert-butyl 3,3-difluoro-2-oxopyrrolidine-1-carboxylate (49b) as an oil, which was used directly in the next reaction.

### Step B—Synthesis of Compound 49c

A mixture of crude tert-butyl 3,3-difluoro-2-oxopyrrolidine-1-carboxylate (49b, 181 mg, 0.82 mmol), lithium hydroxide (9.6 mg, 0.40 mmol) and DMF (0.40 mL) was stirred at 60° C. for 30 min, then at room temperature overnight. To the resultant suspension were added (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 540 μL, 0.19-0.22 mmol, ˜0.35-0.40 M in DMF with 3.0 eq. Et₃N) and HATU (140 mg, 0.36 mmol). The mixture was stirred at room temperature for 20 min. Afterwards, the mixture was solubilized with DMSO (1.0 mL) and subjected to reverse phase column chromatography (10-100% MeCN/H₂O with 0.1% formic acid modifier) to afford tert-butyl (4-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-4-oxobutyl)carbamate (49c) as a solid.

### Step C—Synthesis of Compound 49

A mixture of tert-butyl (4-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-4-oxobutyl)carbamate (49c, 30 mg, 0.045 mmol), TFA (35 μL, 0.45 mmol) and DCM (0.30 mL) was stirred at room temperature for 5 hr. The mixture was then diluted with DMSO and subjected to reverse phase column chromatography (10-70% MeCN/H₂O with 0.1% formic acid modifier) to afford 4-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluorobutanamide (49) as a solid. MS: m/z=557 [M+H]. ¹H NMR (500 MHz, DMSO-d₆) δ 8.30 (s, 1H), 7.81 (d, J=10.9 Hz, 1H), 7.32 (s, 1H), 6.54 (br s, 1H), 5.60 (t, J=5.5 Hz, 1H), 5.42 (s, 2H), 5.25 (d, J=18.6 Hz, 1H), 5.10 (d, J=18.6 Hz, 1H), 3.24-3.11 (m, 2H), 2.81 (t, J=6.9 Hz, 2H), 2.40 (s, 3H), 2.38-2.26 (m, 2H), 2.25-2.13 (m, 2H), 1.96-1.79 (m, 2H), 0.87 (t, J=7.3 Hz, 3H).

### Example 14

### Preparation of Compound 50

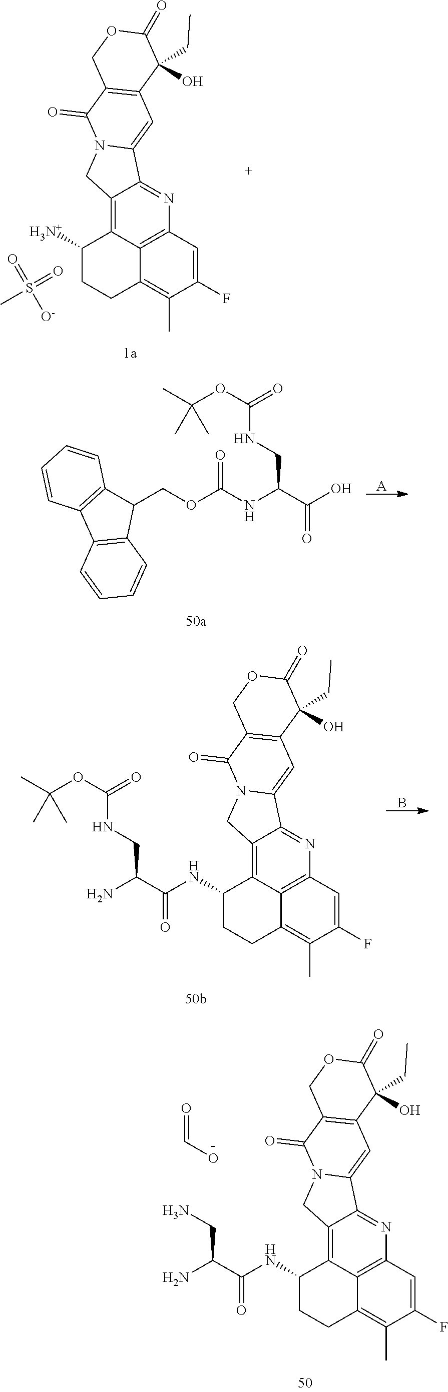

### Step A—Synthesis of Compound 50b

To a stirred mixture of (S)-2-((((9H-fluoren-9-yl)methoxy)carbonyl)amino)-3-((tert-butoxycarbonyl)amino)propanoic acid (50a, 51 mg, 0.12 mmol), Hunig's Base (21 μL, 0.12 mmol), (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 290 μL, 0.10-0.12 mmol, ˜0.35-0.40 M in DMF with 3.0 eq. Et₃N) and DMF (0.30 mL) was added HATU (46 mg, 0.12 mmol). The reaction mixture was stirred at room temperature for 20 min, at which point 4-methylpiperidine (24 μL, 0.20 mmol) was added. After stirring at 40° C. for 2 hr, the reaction mixture was subjected to reverse phase column chromatography (10-50% MeCN/H₂O with 0.1% formic acid modifier) to afford tert-butyl ((S)-2-amino-3-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-oxopropyl)carbamate (50b) as a solid.

### Step B—Synthesis of Compound 50

A mixture of tert-butyl ((S)-2-amino-3-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-oxopropyl)carbamate (50b, 44 mg, 0.071 mmol), TFA (0.11 mL, 1.4 mmol) and DCM (0.30 mL) was stirred at room temperature for 45 min. The mixture was then quenched with N-methylmorpholine (0.16 mL, 1.4 mmol), diluted with water, and subjected to reverse phase column chromatography (10-50% MeCN/H₂O with 0.1% formic acid modifier) to afford(S)-2,3-diamino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)propanamide formate (50) as a solid. MS: m/z=522 [M+H]. ¹H NMR (500 MHz, DMSO-d₆) δ 8.65 (br s, 1H), 8.14 (s, 1H), 7.81 (d, J=10.9 Hz, 1H), 7.32 (s, 1H), 6.67 (br s, 3H), 5.59-5.50 (m, 1H), 5.42 (s, 2H), 5.26 (d, J=18.7 Hz, 1H), 5.20 (d, J=18.8 Hz, 1H), 3.48 (dd, J=8.9, 4.6 Hz, 1H), 3.23-3.16 (m, 2H), 3.08 (dd, J=12.7, 4.6 Hz, 1H), 2.81 (dd, J=12.7, 9.0 Hz, 1H), 2.41 (s, 3H), 2.27-2.18 (m, 1H), 2.18-2.07 (m, 1H), 1.93-1.78 (m, 2H), 0.87 (t, J=7.3 Hz, 3H).

### Example 15

### Preparation of Compound 51

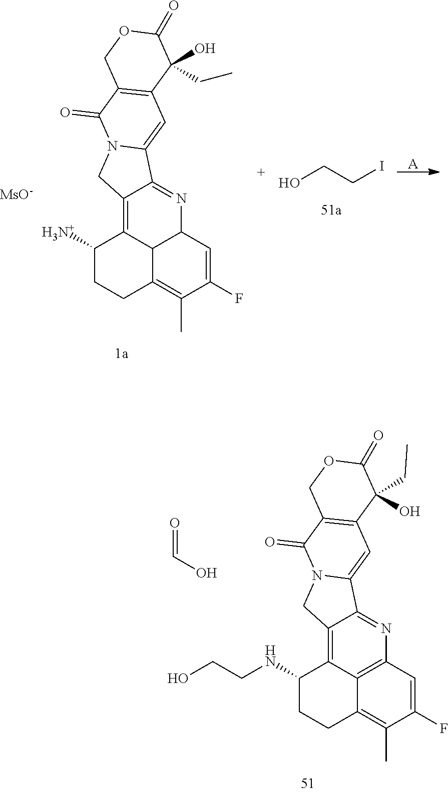

### Step A—Synthesis of Compound 51

A mixture of (1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-aminium methanesulfonate (1a, 270 μL, 0.095-0.11 mmol, ˜0.35-0.40 M in DMF, with 3 eq. TEA), 2-iodoethanol (16 μL, 0.020 mmol), DIPEA (35 μL, 0.20 mmol) and DMF (0.20 mL) was stirred at 40° C. for 3 days. The mixture was then directly purified via reverse phase column chromatography (10-90% MeCN/water with 0.1% formic acid modifier) to afford (1S,9S)-9-ethyl-5-fluoro-9-hydroxy-1-((2-hydroxyethyl)amino)-4-methyl-1,2,3,9,12,15-hexahydro-10H,13H-benzo[de]pyrano[3′,4′: 6,7]indolizino[1,2-b]quinoline-10,13-dione formate (51). MS: m/z=480 [M+H]. ¹H NMR (500 MHz, DMSO-d₆) δ 8.13 (s, 1H), 7.73 (d, J=11.0 Hz, 1H), 7.30 (s, 1H), 6.51 (s, 1H), 5.45-5.31 (m, 4H), 4.33 (s, 1H), 3.58-3.48 (m, 2H), 3.24-3.13 (m, 1H), 3.02 (dt, J=17.2, 5.0 Hz, 1H), 2.88 (dt, J=11.6, 6.2 Hz, 1H), 2.77 (dt, J=11.5, 5.6 Hz, 1H), 2.37 (s, 3H), 2.32-2.23 (m, 1H), 2.11-2.00 (m, 1H), 1.95-1.78 (m, 2H), 0.87 (t, J=7.3 Hz, 3H).

The following compounds of the present disclosure in Table 9 were made using similar methods described in Example 15 with varying reaction times (2 hr to 1 week) and temperatures (23-50° C.), and substituting the appropriate alkyl electrophile reagents:

TABLE 9

| Com- | | MS |
|---|---|---|
| pound | Structure | [M + H] |
| 52 | 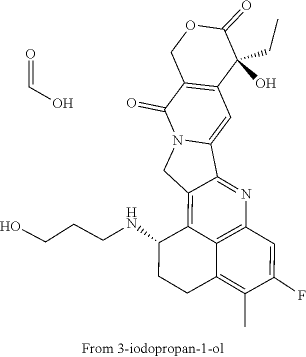 [SMILES: CC[C@@]1(O)C(=O)OCc2c1cc1n(c2=O)Cc2c-1nc1cc(F)c(C)c3c1c2[C@@H](NCCCO)CC3 ; O=CO] | 494 |
| 53 | 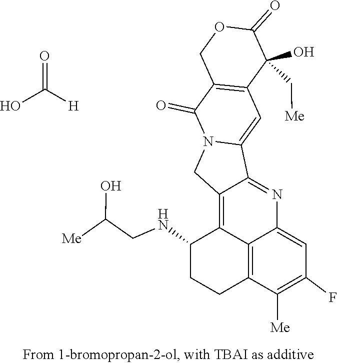 [SMILES: O=CO ; CC[C@@]1(O)C(=O)OCc2c1cc1n(c2=O)Cc2c-1nc1cc(F)c(C)c3c1c2[C@@H](NCC(C)O)CC3] | 494 |
| 54 | 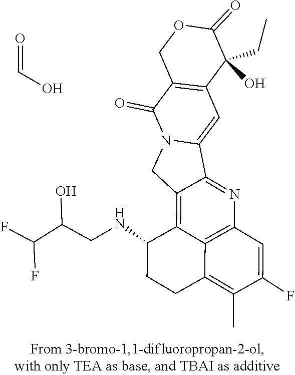 [SMILES: CC[C@@]1(O)C(=O)OCc2c1cc1n(c2=O)Cc2c-1nc1cc(F)c(C)c3c1c2[C@@H](NCC(O)C(F)F)CC3 ; O=CO] | 530 |
| 55 | 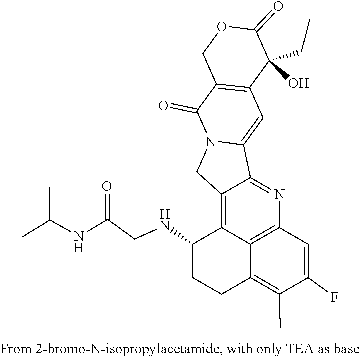 [SMILES: CC[C@@]1(O)C(=O)OCc2c1cc1n(c2=O)Cc2c-1nc1cc(F)c(C)c3c1c2[C@@H](NCC(=O)NC(C)C)CC3] | 535 |
| 56 | 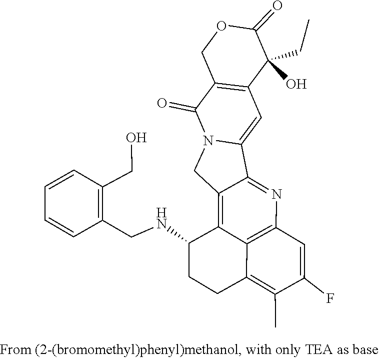 [SMILES: CC[C@@]1(O)C(=O)OCc2c1cc1n(c2=O)Cc2c-1nc1cc(F)c(C)c3c1c2[C@@H](NCc1ccccc1CO)CC3] | 556 |
| 57 | 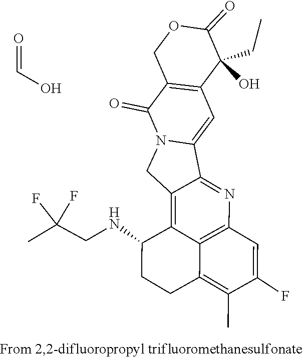 [SMILES: O=CO ; CC[C@@]1(O)C(=O)OCc2c1cc1n(c2=O)Cc2c-1nc1cc(F)c(C)c3c1c2[C@@H](NCC(C)(F)F)CC3] | 514 |
| 58 | 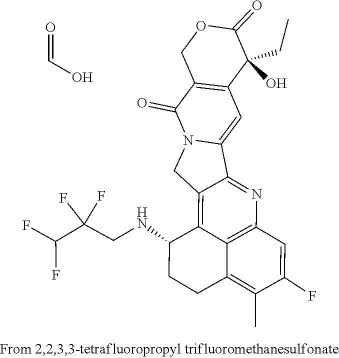 [SMILES: CC[C@@]1(O)C(=O)OCc2c1cc1n(c2=O)Cc2c-1nc1cc(F)c(C)c3c1c2[C@@H](NCC(F)(F)C(F)F)CC3 ; O=CO] | 550 |
| 59 | 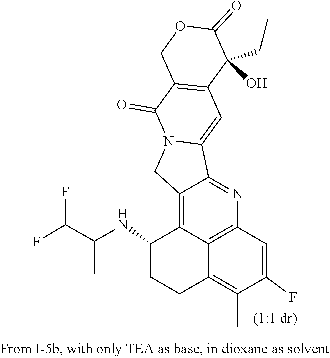 [SMILES: CC[C@@]1(O)C(=O)OCc2c1cc1n(c2=O)Cc2c-1nc1cc(F)c(C)c3c1c2[C@@H](NC(C)C(F)F)CC3] | 514 |

### Example 16

### Preparation of Compound 60

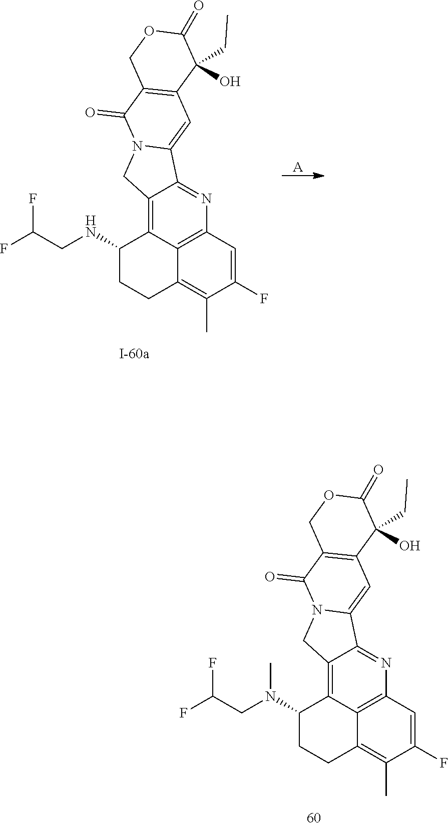

### Step A—Synthesis of Compound 60

At 0° C., to a suspension of (1S,9S)-1-((2,2-difluoroethyl)amino)-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione (I-60a, 17 mg, 0.035 mmol) in MeOH (0.70 mL) was added formaldehyde (37 wt % in water, 26 μL, 0.35 mmol), acetic acid (4.0 μL, 0.070 mmol) and sodium triacetoxyborohydride (15 mg, 0.070 mmol). After 6 h, DCM (0.60 mL) was added to solubilize the mixture, followed by formaldehyde (37% wt in water, 26 μL, 0.35 mmol), acetic acid (4.0 μL, 0.070 mmol) and sodium triacetoxyborohydride (15 mg, 0.070 mmol). The mixture was stirred at room temperature over 3 days. At this point, formaldehyde (37% wt in water, 52 μL, 0.70 mmol), acetic acid (8.0 μL, 0.14 mmol) and sodium triacetoxyborohydride (29 mg, 0.14 mmol) were added, and the mixture was stirred at room temperature for 30 minutes. This step was repeated one more time. The mixture was then subjected to reverse phase column chromatography (30-90% MeCN/H₂O with 0.1% formic acid modifier) to afford (1S,9S)-1-((2,2-difluoroethyl)(methyl)amino)-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione as a solid. MS: m/z=514 [M+H]. ¹H NMR (500 MHz, DMSO-d₆) δ 7.75 (d, J=10.8 Hz, 1H), 7.31 (s, 1H), 6.49 (s, 1H), 6.29 (tt, J=56.3, 4.1 Hz, 1H), 5.42 (s, 2H), 5.37 (d, J=19.6 Hz, 1H), 5.27 (d, J=19.8 Hz, 1H), 4.45 (dd, J=10.9, 4.6 Hz, 1H), 3.07-2.85 (m, 2H), 2.37 (s, 3H), 2.34-2.22 (m, 4H), 2.11-1.99 (m, 1H), 1.94-1.79 (m, 2H), 0.88 (t, J=7.3 Hz, 3H).

### Example 17

CellTiter-Glo® 2.0 Cytotoxicity Assay

### Step 1: Seed 384-Well Plates for Assay (45 μL Per Well) on Day 0

Jeko-1 cells were quickly thawed in a cryo-vial by incubating them in a 37° C. water bath for <1 min until there is just a small bit of ice left in the vial. The vial was promptly removed and wiped down with 70% ethanol. The cells were transferred from the vial to a sterile centrifuge tube containing 8 mL of pre-warmed cell culture medium. The vial was flushed with an additional 1 mL of medium to ensure complete transfer of cells to the centrifuge tube. The cells were then centrifuged at 150×g for 5 minutes. The supernatant was aspirated, and the cell pellet was resuspended in 10-20 mL cell culture medium. Cells were counted using Vi-cell and prepared 6.6×10⁴ cells/ml, 3000 cells/45 μL per well. Then added 45 μL/well of cells into Corning® 384-well Low Flange White Flat Bottom Polystyrene TC-treated Microplates (Corning, Cat #3570) using Standard Cassette Combi (if needed, dispense 1 dummy plate at 20 μL to help normalize Combi, using medium speed). The plates were spun down at 150×g for 30 seconds.

### Step 2: Add Compounds on Day 1

The compound plates and reference compound stock were taken out and allowed to thaw at RT. The tubes were centrifuged at 2000×g for 30 seconds. The 10× Intermediate assay plates (Greiner plate, Cat #781280) were prepared using an Echo liquid handler. Proper buffer (HBSS (Gibco, Cat #14025-092)+10 mM HEPES (Gibco, Cat #15630-080)+0.1% BSA (Sigma, Cat #A9576)) were used to make serial dilutions. Media (no cells) was used for Max_E. Compound (5 μL of 10×) was transferred from intermediate plate to assay plate using a Bravo liquid handler using a very slow speed so the cell monolayer wasn't disturbed. The plates were spun down at 150×g for 30 seconds.

### Step 3: CellTiter-Glo 2.0 Assay (Promega, Cat #G9242) on Day 4 or 5 (CellTiter-Glo Kit Stored at −70° C.)

The CellTiter-Glo® 2.0 Reagent was thawed at 4° C. overnight, taking care not to expose the reagent to temperatures above 25° C.). The kit was equilibrated to RT for approximately 30 minutes. CellTiter-Glo® 2.0 Reagent (20 μl) was added to 50 μl of medium containing cells using Standard Cassette Combi. The contents were mixed for 2-3 minutes on an orbital shaker to induce cell lysis. The plates were spun down at 150×g for 30 seconds. The plates were allowed to incubate at RT for 5 minutes to stabilize the luminescent signal. The luminescence was recorded to calculate an EC₅₀ value, using an integration time of 0.25-1 second per well as a guideline.

Illustrative compounds of the present disclosure were tested in the above assay in Example 15, and results are provided in the Table 9 below:

TABLE 9

| Compound | Jeko-1 EC₅₀ (nM) |
|---|---|
| 1 | 1.4 |
| 2 | 6.6 |
| 3 | 4.1 |
| 4 (peak 2) | 1.2 |
| 5 (peak 1) | 6.7 |
| 6 | 2.9 |
| 7 (peak 1) | 1.7 |
| 8 (peak 2) | 1.6 |
| 9 | 4.0 |
| 10 (peak 1) | 4.4 |
| 11 (peak 2) | 5.3 |
| 12 | 2.0 |
| 13 | 4.2 |
| 14 (peak 1) | 2.8 |
| 15 (peak 2) | 2.9 |
| 16 (peak 1) | 1.6 |
| 17 (peak 2) | 2.0 |
| 18 (peak 1) | 44 |
| 19 (peak 2) | 5.7 |
| 20 | 5.2 |
| 21 | 3.6 |
| 22 (peak 1) | 4.3 |
| 23 (peak 2) | 4.6 |
| 24 | 1.8 |
| 25 | 2.6 |
| 26 | 2.9 |
| 27 | 3.1 |
| 28 | 9.7 |
| 29 | 3.7 |
| 30 | 2.5 |
| 31 | 4.1 |
| 32 | 1.4 |
| 33 | 3.8 |
| 34 (peak 1) | 4.3 |
| 35 (peak 2) | 1.5 |
| 36 (peak 1) | 2.4 |
| 37 (peak 2) | 3.1 |
| 38 | 4.6 |
| 39 (peak 1) | 4.9 |
| 40 (peak 2) | 4.6 |
| 41 | 2.3 |
| 42 | 5.5 |
| 43 | 27 |
| 44 | 78 |
| 45 | 27 |
| 46 | 5.0 |
| 47 | 22 |
| 48 | 8.7 |
| 49 | 14 |
| 50 | 90 |
| 51 | 3.7 |
| 52 | 5.1 |
| 55 | 8.8 |
| 56 | 5.5 |
| 57 | 6.2 |
| 58 | 6.2 |
| 59 | 5.4 |
| 60 | 9.6 |

What is claimed:

1. A compound having a structural Formula I, or a pharmaceutically acceptable salt or solvate thereof: 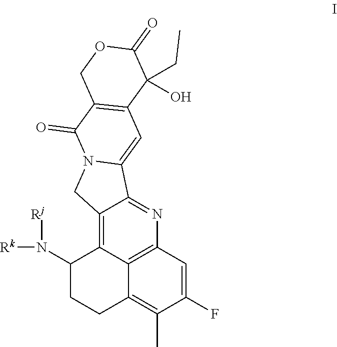
wherein:
R^{k} is selected from hydrogen, —C₁₋₆ alkyl, (CH₂)ₙC(O)NHC₁₋₆ alkyl, (CH₂)ₙC₆₋₁₀ aryl and 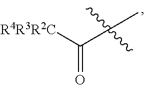
said alkyl and aryl optionally substituted with 1 to 3 groups of hydroxyl, —C₁₋₆ alkylOH, said alkyl further optionally substituted with 1 to 10 groups of halogen;
R^{j} represents hydrogen or C₁₋₆ alkyl, said alkyl optionally substituted with 1 to 10 halogen;
R² and R³ are independently selected from hydrogen, —C₁₋₆ alkyl, OH, —C₁₋₆ alkylOH, halogen, —C₁₋₉haloalkyl, —(CH₂)ₙNH₂, —NHC₁₋₆alkyl, —N(C₁₋₆ alkyl)₂, —C₃₋₆ cycloalkyl, —(CH₂)ₙC₆₋₁₀aryl, and —(CH₂)ₙOC₁₋₆alkyl, that R² and R³ are not both halogen at the same time;
R⁴ is selected from C₁₋₆ alkyl, OH, —C₁₋₆ alkylOH, —CH(OH)C₁₋₆ alkyl, —C₁₋₉haloalkyl, halogen, —C₃₋₆cycloalkyl, —(CH₂)ₙOC₁₋₃alkyl, —(CH₂)ₙOC₁₋₉haloalkyl, —CR^{x}R^{y}C₁₋₆ alkylOH, —(CH₂)ₙNH₂, —NHC₁₋₆ alkyl, —N(C₁₋₆ alkyl)₂, and —NHC(O)C₁₋₆alkylNH₂;
R^{x} and R^{y} represent C₁₋₃ alkylene that combine to form a C₃₋₆cycloalkyl, or spirocycloalkyl;
each n independently represents 0, 1, 2, or 3.

2. The compound according to claim 1, or a pharmaceutically acceptable salt or solvate thereof wherein any hydrogen atom in an alkyl of R², R³ and/or R⁴ is not deuterated.

3. The compound according to claim 1, or a pharmaceutically acceptable salt or solvate thereof wherein R² is selected from hydrogen, C₁₋₆ alkyl, CH₂OC₁₋₆alkyl, —CH₂OH, —O(CH₂)₂OH, —CH₂F, —CHF₂, —CF₃, —(CH₂)phenyl, cyclopropyl, cyclobutyl, cyclopentyl, cyclohexyl, —(CH₂)ₙNH₂, —NHCH₃, and —N(CH₃)₂.

4. The compound according to claim 1, or a pharmaceutically acceptable salt or solvate thereof wherein R² is selected from hydrogen, C₁₋₆ alkyl, (CH₂)ₙOCH₃, —OH, —CH₂OH, —CH₂F, —CHF₂, —CF₃, and —NH₂.

5. The compound according to claim 1, or a pharmaceutically acceptable salt or solvate thereof wherein R³ is selected from hydrogen, C₁₋₆ alkyl, CH₂OC₁₋₆alkyl, —CH₂OH, —(CH₂)₂OH, —CH₂F, —CHF₂, —CF₃, —(CH₂)phenyl, cyclopropyl, cyclobutyl, cyclopentyl, cyclohexyl, —(CH₂)ₙNH₂, —NHCH₃, and —N(CH₃)₂.

6. The compound according to claim 1, or a pharmaceutically acceptable salt or solvate thereof wherein R³ is selected from hydrogen, C₁₋₆ alkyl, (CH₂)ₙOCH₃, —OH, —CH₂OH, —CH₂F, —CHF₂, —CF₃, and —NH₂.

7. The compound according to claim 1, or a pharmaceutically acceptable salt or solvate thereof wherein one of R² and R³ is selected from —OH —CH₂OH, —(CH₂)₂OH, and (CH₂)ₙNH₂ and the other is selected from hydrogen, CH₂phenyl, and C₁₋₆alkyl.

8. The compound according to claim 1, or a pharmaceutically acceptable salt or solvate thereof wherein R⁴ is selected from CH₃, OH, —CH₂OH, —(CH₂)₂OH, —CH(CH₃)OH, —CH₂F, —CHF₂, —CF₃, (CH₂)ₙOCHF₂, cyclopropyl, —(CH₂)ₙNH₂, and -cyclopropylCH₂OH, —NHC(O)CH(CH₃)NH₂.

9. The compound according to claim 1, or a pharmaceutically acceptable salt or solvate thereof wherein R⁴ is OH, —(CH₂)ₙOH, —CH(CH₃)OH, —(CH₂)ₙNH₂, or R⁴ is —CR^{x}R^{y}C₁₋₆ alkylOH, wherein R^{x} and R^{y} are alkylene substituents that combine to form cyclopropyl.

10. The compound according to claim 1, or a pharmaceutically acceptable salt or solvate thereof wherein one of R² and R³ is —OH or —C₁₋₆alkylOH and the other is selected from hydrogen, CH₂phenyl and C₁₋₆alkyl, and R⁴ is selected from CH₃, OH, —C₁₋₆alkylOH, —C₁₋₃haloalkyl, —CR^{x}R^{y}C₁₋₆alkylOH, —(CH₂)ₙNH₂, —NHC₁₋₆ alkyl, —N(C₁₋₆ alkyl)₂, and —NHC(O)C₁₋₆alkylNH₂.

11. The compound according to claim 10, or a pharmaceutically acceptable salt or solvate thereof wherein one of R² and R³ is —OH or —C₁₋₆alkylOH and the other is selected from hydrogen, CH₂phenyl, and C₁₋₆ alkyl and R⁴ is selected from —CH₂OH, —(CH₂)₂OH, —CH(CH₃)OH, —CH₂F, —CHF₂, —CF₃ and —(CH₂)ₙNH₂.

12. The compound according to claim 1, or a pharmaceutically acceptable salt or solvate thereof wherein one of R² and R³ is —(CH₂)ₙNH₂, —NHCH₃, or —N(CH₃)₂ and the other is selected from hydrogen, CH₂phenyl and C₁₋₆alkyl and R⁴ is selected from CH₃, OH, —CH₂OH, —(CH₂)₂OH, —C₁₋₃haloalkyl, —CR^{x}R^{y}C₁₋₆ alkylOH, —(CH₂)ₙNH₂, —NHC₁₋₆ alkyl, —N(C₁₋₆ alkyl)₂, and —NHC(O)C₁₋₆alkylNH₂.

13. The compound according to claim 12, or a pharmaceutically acceptable salt or solvate thereof, wherein one of R² and R³ is (CH₂)ₙNH₂, and the other is selected from hydrogen, CH₂phenyl and C₁₋₆alkyl and R⁴ is selected from R⁴ is selected from —CH₂OH, —(CH₂)₂OH, —CH(CH₃)OH, —CH₂F, —CHF₂, —CF₃ and —(CH₂)ₙNH₂.

14. The compound according to claim 1, or a pharmaceutically acceptable salt or solvate thereof represented by structural Formula II: 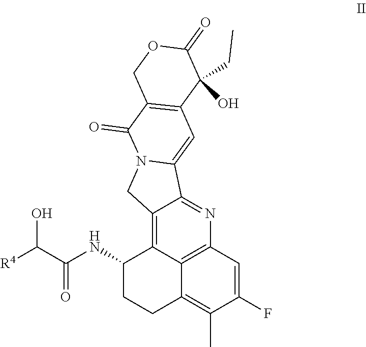
wherein R⁴ is selected from —CH₂OH, —(CH₂)₂OH, —CH(CH₃)OH, —CH₂F, —CHF₂, —CF₃ and —(CH₂)ₙNH₂.

15. The compound according to claim 1, or a pharmaceutically acceptable salt or solvate thereof represented by structural Formula III: 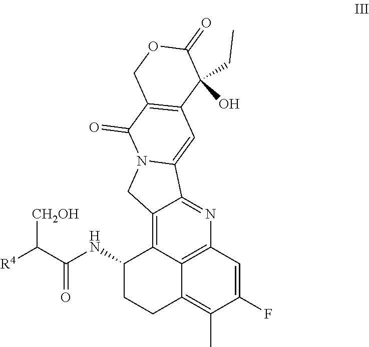
wherein R⁴ is selected from CH₃, —CH₂OH, —(CH₂)₂OH, —CH(CH₃)OH, —CH₂F, —CHF₂, —CF₃ and —(CH₂)ₙNH₂.

16. The compound according to claim 1, or a pharmaceutically acceptable salt or solvate thereof represented by structural Formula IV: 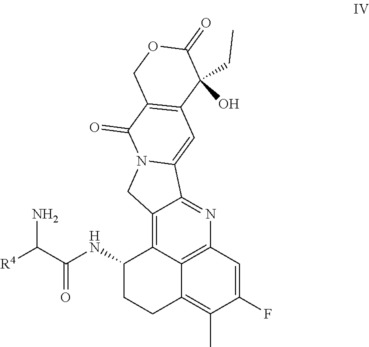
wherein R⁴ is —CH₂F, —CHF₂, —CF₃, or (CH₂)ₙNH₂.

17. A compound, or a pharmaceutically acceptable salt or solvate thereof which is:
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxybutanamide;
(R)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxybutanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxybutanamide;
(S)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxybutanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxy-3,3-dimethylbutanamide;
(R)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxy-3,3-dimethylbutanamide;
(S)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,4-dihydroxy-3,3-dimethylbutanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,3-dihydroxy-2-methylpropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,3-dihydroxy-2-methylpropanamide;
N-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,3-dihydroxy-2-methylpropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,3-dihydroxy-2-methylpropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,3-dihydroxy-2-methylpropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-(hydroxymethyl)-2-methylpropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-(hydroxymethyl)-2-methylpropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluoro-3-hydroxybutanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluoro-3-hydroxybutanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluoro-3-hydroxybutanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluoro-3-hydroxybutanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluoro-3-hydroxypropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluoro-3-hydroxypropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-(hydroxymethyl)propenamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-(hydroxymethyl)propenamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-(hydroxymethyl)propenamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-(hydroxymethyl)propenamide;
1-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-aminium;
1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-aminium;
1-((-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-aminium;
1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-aminium;
3-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,1,1-trifluoro-3-oxopropan-2-aminium;
3-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,1,1-trifluoro-3-oxopropan-2-aminium;
((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,1,1-trifluoro-3-oxopropan-2-aminium;
(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-1,1,1-trifluoro-3-oxopropan-2-aminium;
2-amino-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-methylpropanamide;
2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-methylpropanamide;
2-amino-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-methylpropanamide;
2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoro-2-methylpropanamide;
2-amino-2-benzyl-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoropropanamide;
2-amino-2-benzyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoropropanamide;
2-amino-2-benzyl-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoropropanamide;
2-amino-2-benzyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3,3,3-trifluoropropanamide;
2-amino-2-cyclopropyl-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)propenamide;
2-amino-2-cyclopropyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)propenamide;
4-(difluoromethoxy)-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxybutanamide;
(R)-4-(difluoromethoxy)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxybutanamide;
2-cyclopropyl-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
2-cyclopropyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
2-cyclopropyl-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
2-cyclopropyl-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2,2-dimethylpropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2,2-dimethylpropanamide;
(S)-N-(-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxybutanamide;
(S)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxybutanamide;
N-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxybutanamide;
(R)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxybutanamide;
N-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
1-(2,3-dihydroxypropanamido)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-7-ium hexafluorophosphate (V);
(1S,9S)-1-((S)-2,3-dihydroxypropanamido)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-7-ium hexafluorophosphate (V);
1-(2,3-dihydroxypropanamido)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-7-ium hexafluorophosphate (V);
(1S,9S)-1-((R)-2,3-dihydroxypropanamido)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-7-ium hexafluorophosphate (V);
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxy-2-(1-(hydroxymethyl)cyclopropyl)acetamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-hydroxy-2-(1-(hydroxymethyl)cyclopropyl)acetamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methoxybutanamide;
(R)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methoxybutanamide;
(S)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methoxybutanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-methylpropanamide;
(S)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-methylpropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-methylpropanamide;
(R)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxy-2-methylpropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxypropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxypropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxypropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxypropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxy-2-methylpropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxy-2-methylpropanamide;
(1S,9S)-9-ethyl-5-fluoro-9-hydroxy-1-((2-hydroxypropyl)amino)-4-methyl-1,2,3,9,12,15-hexahydro-10H,13H-benzo[de]pyrano[3′,4′: 6,7]indolizino[1,2-b]quinoline-10,13-dione;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxy-2-methylpropanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-fluoro-3-hydroxy-2-methylpropanamide;
N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2,2-dimethylbutanamide;
N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2,2-dimethylbutanamide;
2-(difluoromethyl)-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methylbutanamide;
2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methylbutanamide;
2-(difluoromethyl)-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methylbutanamide;
2-(difluoromethyl)-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-4-hydroxy-2-methylbutanamide;
2-amino-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-methylpropanamide;
2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2-methylpropanamide;
1-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-4-methyl-1-oxopentan-2-aminium;
1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-4-methyl-1-oxopentan-2-aminium;
3-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-hydroxy-3-oxopropan-1-aminium;
(R)-3-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-hydroxy-3-oxopropan-1-aminium;
3-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-hydroxy-3-oxopropan-1-aminium;
(S)-3-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-2-hydroxy-3-oxopropan-1-aminium;
2-amino-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
(S)-2-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-3-hydroxypropanamide;
1-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-hydroxy-1-oxopropan-2-aminium;
(R)-1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-hydroxy-1-oxopropan-2-aminium;
1-((1-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-yl)amino)-1-oxopropan-2-aminium;
(2R)-1-((1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-yl)amino)-1-oxopropan-2-aminium;
(2S)-1-((1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3,3-difluoro-2-methyl-1-oxopropan-2-yl)amino)-1-oxopropan-2-aminium;
1-((9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-methoxy-2-methyl-1-oxopropan-2-aminium;
1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-methoxy-2-methyl-1-oxopropan-2-aminium;
1-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-3-methoxy-2-methyl-1-oxopropan-2-aminium;
4-amino-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluorobutanamide;
4-amino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)-2,2-difluorobutanamide;
2,3-diamino-N-(9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)propanamide;
(S)-2,3-diamino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)propanamide;
(R)-2,3-diamino-N-((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)propanamide;
(1S,9S)-9-ethyl-5-fluoro-9-hydroxy-1-((2-hydroxyethyl)amino)-4-methyl-1,2,3,9,12,15-hexahydro-10H,13H-benzo[de]pyrano[3′,4′: 6,7]indolizino[1,2-b]quinoline-10,13-dione;
(1S,9S)-9-ethyl-5-fluoro-9-hydroxy-1-((3-hydroxypropyl)amino)-4-methyl-1,2,3,9,12,15-hexahydro-10H,13H-benzo[de]pyrano[3′,4′: 6,7]indolizino[1,2-b]quinoline-10,13-dione;
(1S,9S)-9-ethyl-5-fluoro-9-hydroxy-1-((1-hydroxypropan-2-yl)amino)-4-methyl-1,2,3,9,12,15-hexahydro-10H,13H-benzo[de]pyrano[3′,4′: 6,7]indolizino[1,2-b]quinoline-10,13-dione;
(1S,9S)-1-((3,3-difluoro-2-hydroxypropyl)amino)-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione;
2-(((1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl)amino)-N-isopropylacetamide;
(1S,9S)-9-ethyl-5-fluoro-9-hydroxy-1-((2-(hydroxymethyl)benzyl)amino)-4-methyl-1,2,3,9,12,15-hexahydro-10H,13H-benzo[de]pyrano[3′,4′: 6,7]indolizino[1,2-b]quinoline-10,13-dione;
(1S,9S)-1-((2,2-difluoropropyl)amino)-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione;
(1S,9S)-9-ethyl-5-fluoro-9-hydroxy-4-methyl-1-((2,2,3,3-tetrafluoropropyl)amino)-1,2,3,9,12,15-hexahydro-10H,13H-benzo[de]pyrano[3′,4′: 6,7]indolizino[1,2-b]quinoline-10,13-dione;
(1S,9S)-1-((1,1-difluoropropan-2-yl)amino)-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione; or
(1S,9S)-1-((2,2-difluoroethyl)(methyl)amino)-9-ethyl-5-fluoro-9-hydroxy-4-methyl-1,2,3,9,12,15-hexahydro-10H,13H-benzo[de]pyrano[3′,4′: 6,7]indolizino[1,2-b]quinoline-10,13-dione.

18. The compound according to claim 17, or a pharmaceutically acceptable salt or solvate thereof which is:
Structure

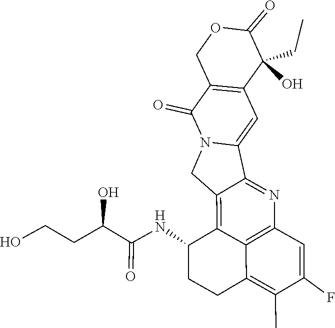 [SMILES: CC[C@@]1(O)C(=O)OCc2c1cc1n(c2=O)Cc2c-1nc1cc(F)c(C)c3c1c2[C@@H](NC(=O)[C@H](O)CCO)CC3]

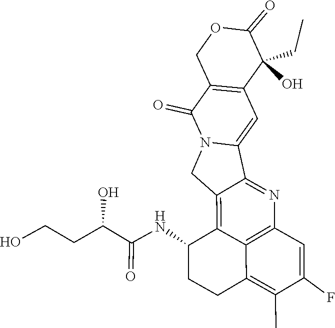 [SMILES: CC[C@@]1(O)C(=O)OCc2c1cc1n(c2=O)Cc2c-1nc1cc(F)c(C)c3c1c2[C@@H](NC(=O)[C@@H](O)CCO)CC3]

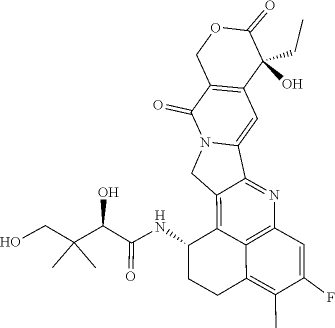 [SMILES: CC[C@@]1(O)C(=O)OCc2c1cc1n(c2=O)Cc2c-1nc1cc(F)c(C)c3c1c2[C@@H](NC(=O)[C@H](O)C(C)(C)CO)CC3]

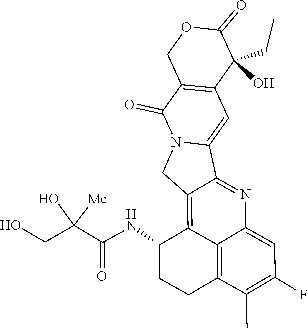 [SMILES: CC[C@@]1(O)C(=O)OCc2c1cc1n(c2=O)Cc2c-1nc1cc(F)c(C)c3c1c2[C@@H](NC(=O)C(C)(O)CO)CC3]

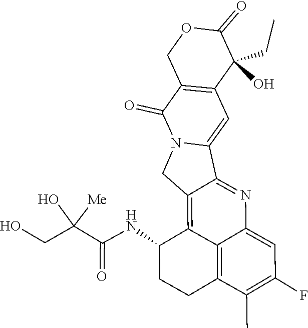 [SMILES: CC[C@@]1(O)C(=O)OCc2c1cc1n(c2=O)Cc2c-1nc1cc(F)c(C)c3c1c2[C@@H](NC(=O)C(C)(O)CO)CC3]

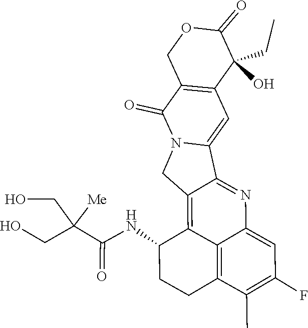 [SMILES: CC[C@@]1(O)C(=O)OCc2c1cc1n(c2=O)Cc2c-1nc1cc(F)c(C)c3c1c2[C@@H](NC(=O)C(C)(CO)CO)CC3]

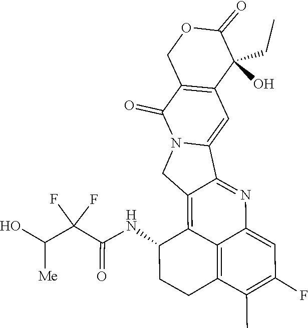 [SMILES: CC[C@@]1(O)C(=O)OCc2c1cc1n(c2=O)Cc2c-1nc1cc(F)c(C)c3c1c2[C@@H](NC(=O)C(F)(F)C(C)O)CC3]

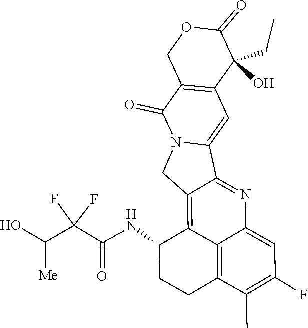 [SMILES: CC[C@@]1(O)C(=O)OCc2c1cc1n(c2=O)Cc2c-1nc1cc(F)c(C)c3c1c2[C@@H](NC(=O)C(F)(F)C(C)O)CC3]

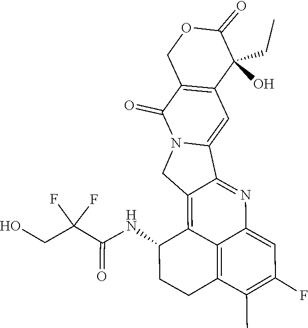 [SMILES: CC[C@@]1(O)C(=O)OCc2c1cc1n(c2=O)Cc2c-1nc1cc(F)c(C)c3c1c2[C@@H](NC(=O)C(F)(F)CO)CC3]

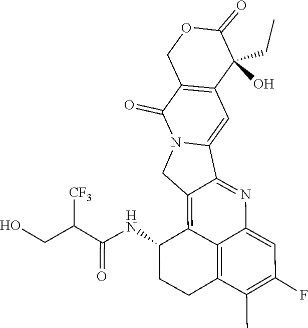 [SMILES: CC[C@@]1(O)C(=O)OCc2c1cc1n(c2=O)Cc2c-1nc1cc(F)c(C)c3c1c2[C@@H](NC(=O)C(CO)C(F)(F)F)CC3]

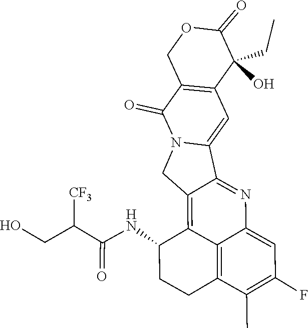 [SMILES: CC[C@@]1(O)C(=O)OCc2c1cc1n(c2=O)Cc2c-1nc1cc(F)c(C)c3c1c2[C@@H](NC(=O)C(CO)C(F)(F)F)CC3]

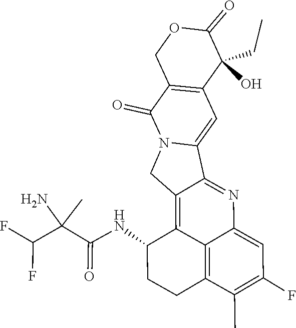 [SMILES: CC[C@@]1(O)C(=O)OCc2c1cc1n(c2=O)Cc2c-1nc1cc(F)c(C)c3c1c2[C@@H](NC(=O)C(C)(N)C(F)F)CC3]

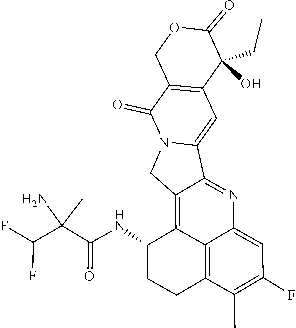 [SMILES: CC[C@@]1(O)C(=O)OCc2c1cc1n(c2=O)Cc2c-1nc1cc(F)c(C)c3c1c2[C@@H](NC(=O)C(C)(N)C(F)F)CC3]

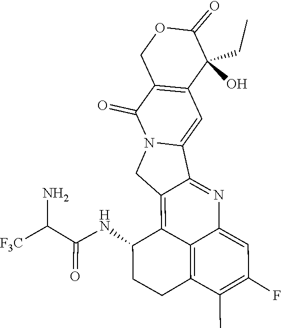 [SMILES: CC[C@@]1(O)C(=O)OCc2c1cc1n(c2=O)Cc2c-1nc1cc(F)c(C)c3c1c2[C@@H](NC(=O)C(N)C(F)(F)F)CC3]

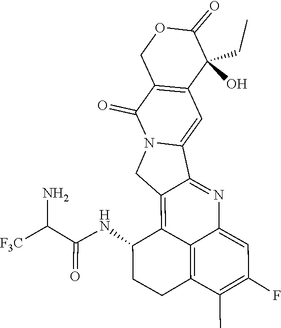 [SMILES: CC[C@@]1(O)C(=O)OCc2c1cc1n(c2=O)Cc2c-1nc1cc(F)c(C)c3c1c2[C@@H](NC(=O)C(N)C(F)(F)F)CC3]

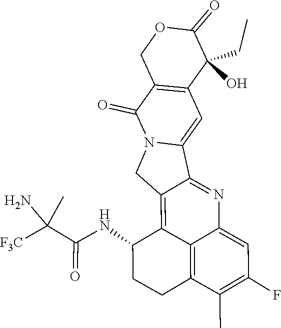 [SMILES: CC[C@@]1(O)C(=O)OCc2c1cc1n(c2=O)Cc2c-1nc1cc(F)c(C)c3c1c2[C@@H](NC(=O)C(C)(N)C(F)(F)F)CC3]

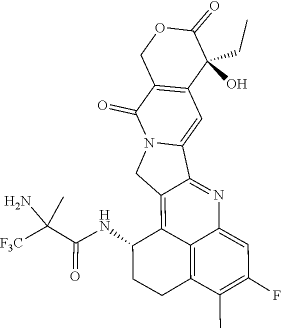 [SMILES: CC[C@@]1(O)C(=O)OCc2c1cc1n(c2=O)Cc2c-1nc1cc(F)c(C)c3c1c2[C@@H](NC(=O)C(C)(N)C(F)(F)F)CC3]

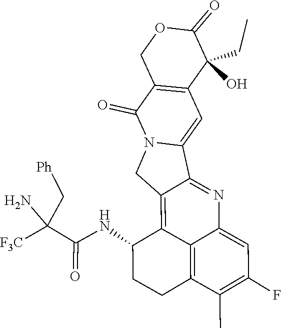 [SMILES: CC[C@@]1(O)C(=O)OCc2c1cc1n(c2=O)Cc2c-1nc1cc(F)c(C)c3c1c2[C@@H](NC(=O)C(N)(Cc1ccccc1)C(F)(F)F)CC3]

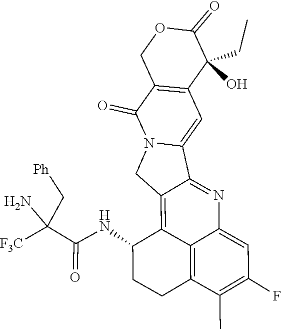 [SMILES: CC[C@@]1(O)C(=O)OCc2c1cc1n(c2=O)Cc2c-1nc1cc(F)c(C)c3c1c2[C@@H](NC(=O)C(N)(Cc1ccccc1)C(F)(F)F)CC3]

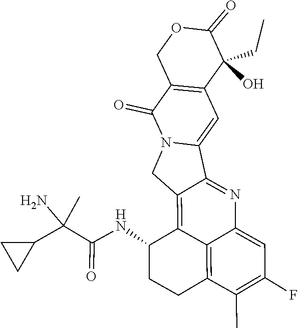 [SMILES: CC[C@@]1(O)C(=O)OCc2c1cc1n(c2=O)Cc2c-1nc1cc(F)c(C)c3c1c2[C@@H](NC(=O)C(C)(N)C1CC1)CC3]

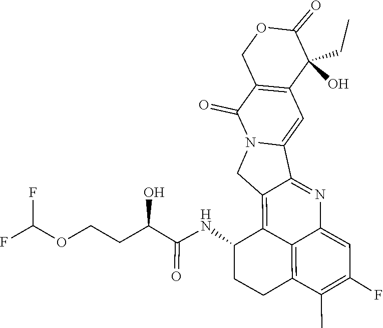 [SMILES: CC[C@@]1(O)C(=O)OCc2c1cc1n(c2=O)Cc2c-1nc1cc(F)c(C)c3c1c2[C@@H](NC(=O)[C@H](O)CCOC(F)F)CC3]

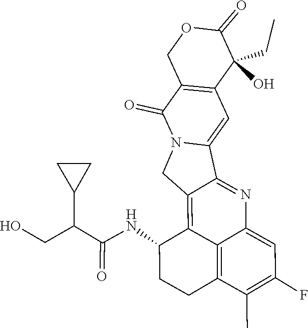 [SMILES: CC[C@@]1(O)C(=O)OCc2c1cc1n(c2=O)Cc2c-1nc1cc(F)c(C)c3c1c2[C@@H](NC(=O)C(CO)C1CC1)CC3]

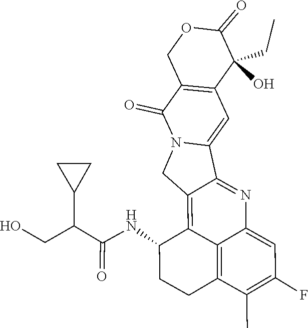 [SMILES: CC[C@@]1(O)C(=O)OCc2c1cc1n(c2=O)Cc2c-1nc1cc(F)c(C)c3c1c2[C@@H](NC(=O)C(CO)C1CC1)CC3]

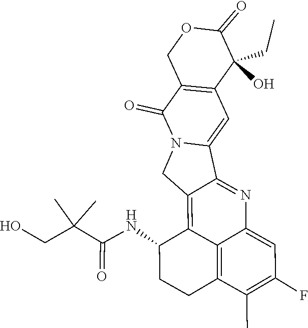 [SMILES: CC[C@@]1(O)C(=O)OCc2c1cc1n(c2=O)Cc2c-1nc1cc(F)c(C)c3c1c2[C@@H](NC(=O)C(C)(C)CO)CC3]

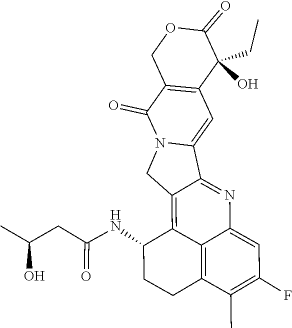 [SMILES: CC[C@@]1(O)C(=O)OCc2c1cc1n(c2=O)Cc2c-1nc1cc(F)c(C)c3c1c2[C@@H](NC(=O)C[C@H](C)O)CC3]

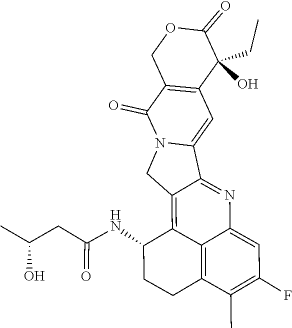 [SMILES: CC[C@@]1(O)C(=O)OCc2c1cc1n(c2=O)Cc2c-1nc1cc(F)c(C)c3c1c2[C@@H](NC(=O)C[C@@H](C)O)CC3]

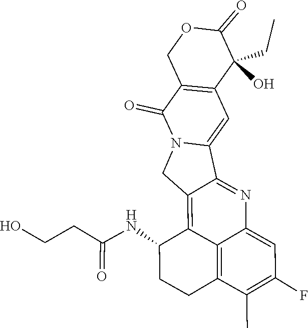 [SMILES: CC[C@@]1(O)C(=O)OCc2c1cc1n(c2=O)Cc2c-1nc1cc(F)c(C)c3c1c2[C@@H](NC(=O)CCO)CC3]

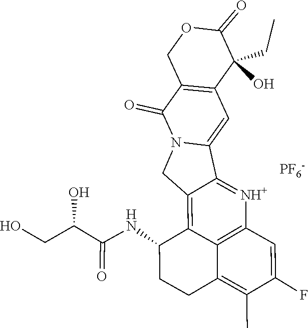

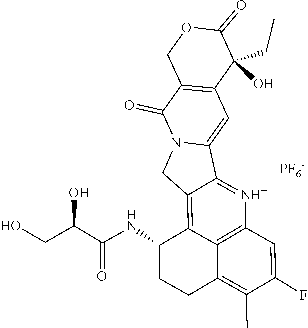

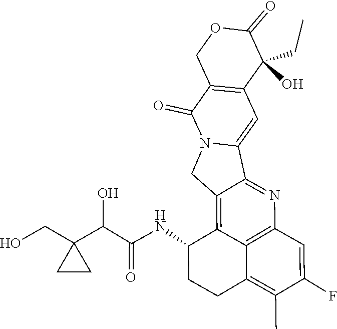 [SMILES: CC[C@@]1(O)C(=O)OCc2c1cc1n(c2=O)Cc2c-1nc1cc(F)c(C)c3c1c2[C@@H](NC(=O)C(O)C1(CO)CC1)CC3]

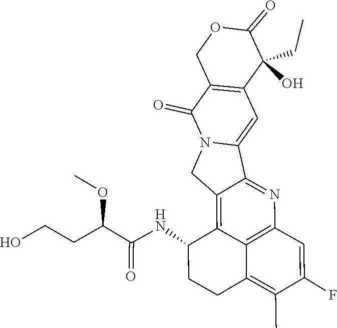 [SMILES: CC[C@@]1(O)C(=O)OCc2c1cc1n(c2=O)Cc2c-1nc1cc(F)c(C)c3c1c2[C@@H](NC(=O)[C@@H](CCO)OC)CC3]

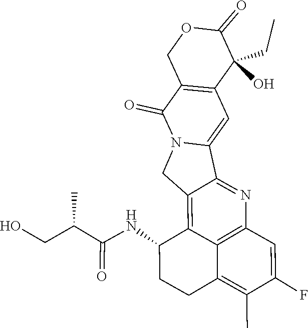 [SMILES: CC[C@@]1(O)C(=O)OCc2c1cc1n(c2=O)Cc2c-1nc1cc(F)c(C)c3c1c2[C@@H](NC(=O)[C@@H](C)CO)CC3]

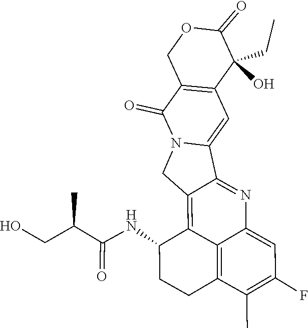 [SMILES: CC[C@@]1(O)C(=O)OCc2c1cc1n(c2=O)Cc2c-1nc1cc(F)c(C)c3c1c2[C@@H](NC(=O)[C@H](C)CO)CC3]

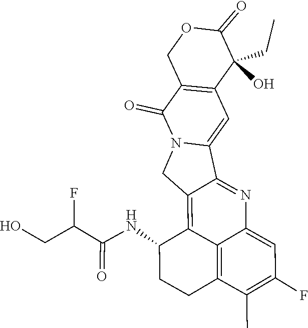 [SMILES: CC[C@@]1(O)C(=O)OCc2c1cc1n(c2=O)Cc2c-1nc1cc(F)c(C)c3c1c2[C@@H](NC(=O)C(F)CO)CC3]

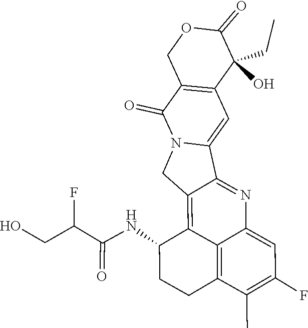 [SMILES: CC[C@@]1(O)C(=O)OCc2c1cc1n(c2=O)Cc2c-1nc1cc(F)c(C)c3c1c2[C@@H](NC(=O)C(F)CO)CC3]

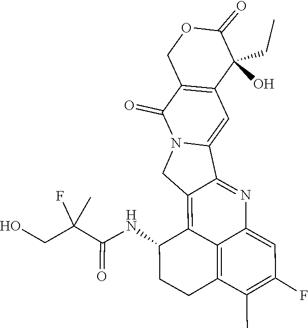 [SMILES: CC[C@@]1(O)C(=O)OCc2c1cc1n(c2=O)Cc2c-1nc1cc(F)c(C)c3c1c2[C@@H](NC(=O)C(C)(F)CO)CC3]

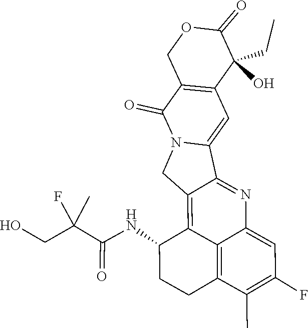 [SMILES: CC[C@@]1(O)C(=O)OCc2c1cc1n(c2=O)Cc2c-1nc1cc(F)c(C)c3c1c2[C@@H](NC(=O)C(C)(F)CO)CC3]

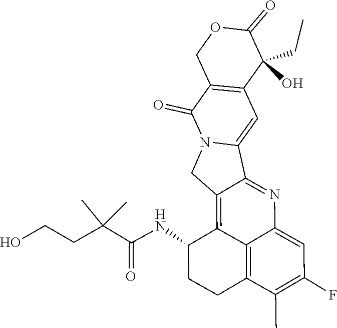 [SMILES: CC[C@@]1(O)C(=O)OCc2c1cc1n(c2=O)Cc2c-1nc1cc(F)c(C)c3c1c2[C@@H](NC(=O)C(C)(C)CCO)CC3]

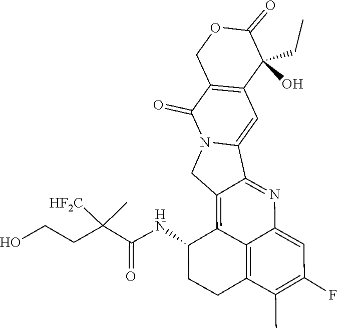

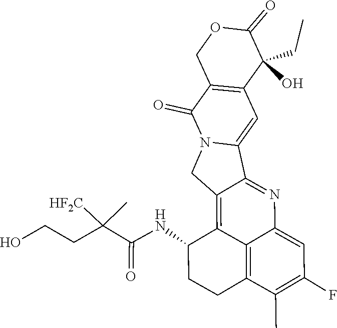

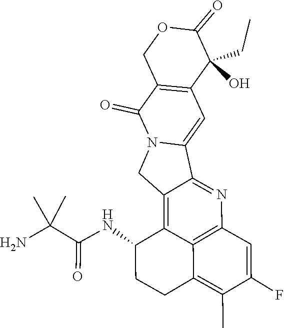 [SMILES: CC[C@@]1(O)C(=O)OCc2c1cc1n(c2=O)Cc2c-1nc1cc(F)c(C)c3c1c2[C@@H](NC(=O)C(C)(C)N)CC3]

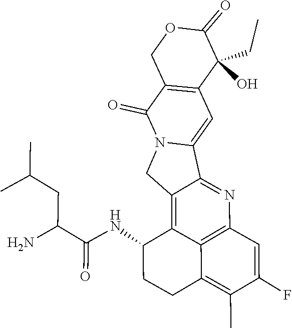 [SMILES: CC[C@@]1(O)C(=O)OCc2c1cc1n(c2=O)Cc2c-1nc1cc(F)c(C)c3c1c2[C@@H](NC(=O)C(N)CC(C)C)CC3]

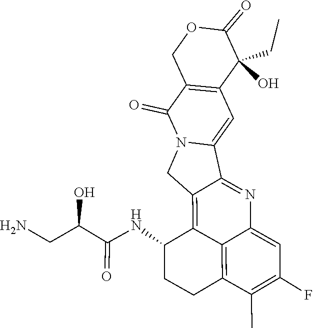 [SMILES: CC[C@@]1(O)C(=O)OCc2c1cc1n(c2=O)Cc2c-1nc1cc(F)c(C)c3c1c2[C@@H](NC(=O)[C@H](O)CN)CC3]

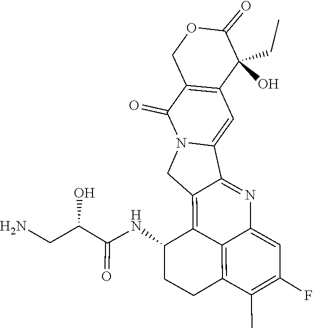 [SMILES: CC[C@@]1(O)C(=O)OCc2c1cc1n(c2=O)Cc2c-1nc1cc(F)c(C)c3c1c2[C@@H](NC(=O)[C@@H](O)CN)CC3]

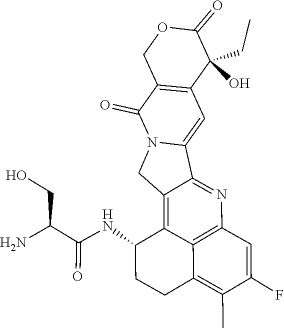 [SMILES: CC[C@@]1(O)C(=O)OCc2c1cc1n(c2=O)Cc2c-1nc1cc(F)c(C)c3c1c2[C@@H](NC(=O)[C@@H](N)CO)CC3]

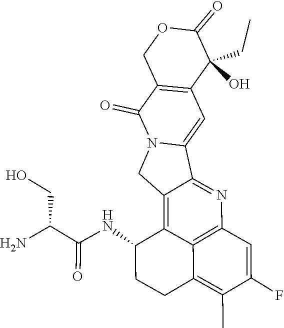 [SMILES: CC[C@@]1(O)C(=O)OCc2c1cc1n(c2=O)Cc2c-1nc1cc(F)c(C)c3c1c2[C@@H](NC(=O)[C@H](N)CO)CC3]

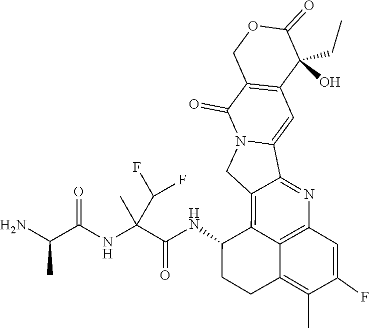 [SMILES: CC[C@@]1(O)C(=O)OCc2c1cc1n(c2=O)Cc2c-1nc1cc(F)c(C)c3c1c2[C@@H](NC(=O)C(C)(NC(=O)[C@@H](C)N)C(F)F)CC3]

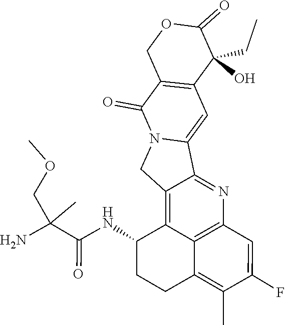 [SMILES: CC[C@@]1(O)C(=O)OCc2c1cc1n(c2=O)Cc2c-1nc1cc(F)c(C)c3c1c2[C@@H](NC(=O)C(C)(N)COC)CC3]

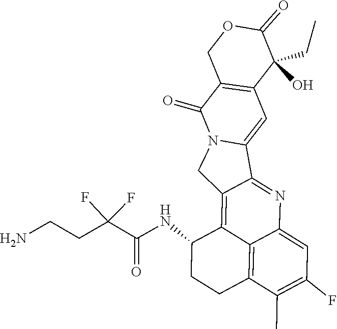 [SMILES: CC[C@@]1(O)C(=O)OCc2c1cc1n(c2=O)Cc2c-1nc1cc(F)c(C)c3c1c2[C@@H](NC(=O)C(F)(F)CCN)CC3]

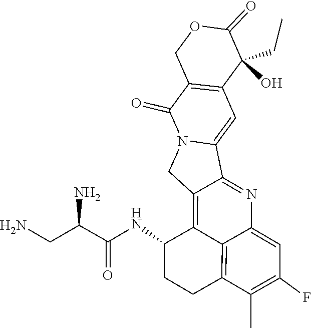 [SMILES: CC[C@@]1(O)C(=O)OCc2c1cc1n(c2=O)Cc2c-1nc1cc(F)c(C)c3c1c2[C@@H](NC(=O)[C@H](N)CN)CC3]

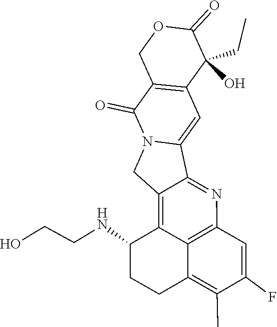 [SMILES: CC[C@@]1(O)C(=O)OCc2c1cc1n(c2=O)Cc2c-1nc1cc(F)c(C)c3c1c2[C@@H](NCCO)CC3]

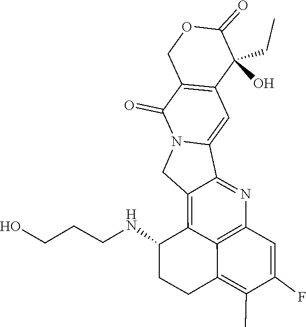 [SMILES: CC[C@@]1(O)C(=O)OCc2c1cc1n(c2=O)Cc2c-1nc1cc(F)c(C)c3c1c2[C@@H](NCCCO)CC3]

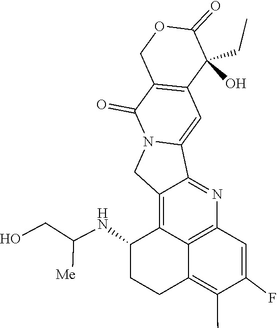 [SMILES: CC[C@@]1(O)C(=O)OCc2c1cc1n(c2=O)Cc2c-1nc1cc(F)c(C)c3c1c2[C@@H](NC(C)CO)CC3]

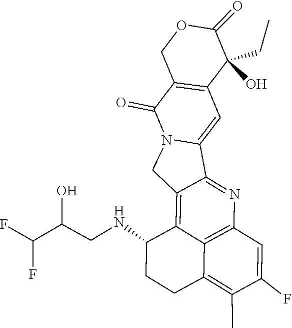 [SMILES: CC[C@@]1(O)C(=O)OCc2c1cc1n(c2=O)Cc2c-1nc1cc(F)c(C)c3c1c2[C@@H](NCC(O)C(F)F)CC3]

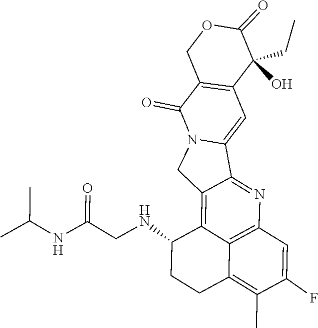 [SMILES: CC[C@@]1(O)C(=O)OCc2c1cc1n(c2=O)Cc2c-1nc1cc(F)c(C)c3c1c2[C@@H](NCC(=O)NC(C)C)CC3]

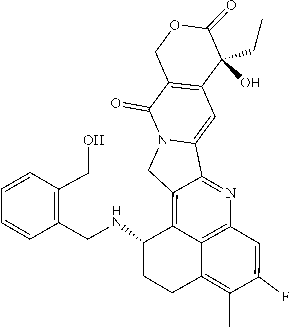 [SMILES: CC[C@@]1(O)C(=O)OCc2c1cc1n(c2=O)Cc2c-1nc1cc(F)c(C)c3c1c2[C@@H](NCc1ccccc1CO)CC3]

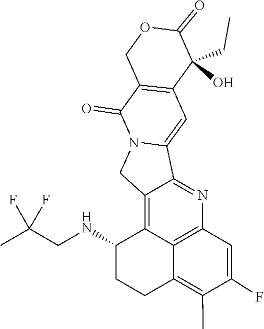 [SMILES: CC[C@@]1(O)C(=O)OCc2c1cc1n(c2=O)Cc2c-1nc1cc(F)c(C)c3c1c2[C@@H](NCC(C)(F)F)CC3]

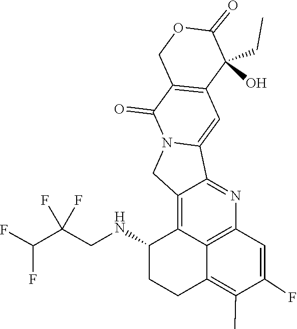 [SMILES: CC[C@@]1(O)C(=O)OCc2c1cc1n(c2=O)Cc2c-1nc1cc(F)c(C)c3c1c2[C@@H](NCC(F)(F)C(F)F)CC3]

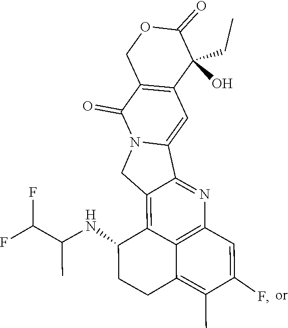 [SMILES: CC[C@@]1(O)C(=O)OCc2c1cc1n(c2=O)Cc2c-1nc1cc(F)c(C)c3c1c2[C@@H](NC(C)C(F)F)CC3 ; "or"]

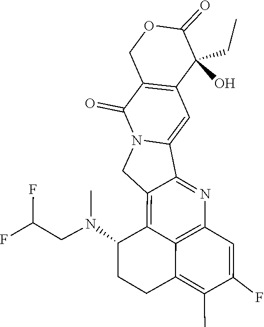 [SMILES: CC[C@@]1(O)C(=O)OCc2c1cc1n(c2=O)Cc2c-1nc1cc(F)c(C)c3c1c2[C@@H](N(C)CC(F)F)CC3]

19. A pharmaceutical composition comprising a compound of claim 1, or a pharmaceutically acceptable salt or solvate thereof, and a pharmaceutically acceptable carrier.

20. Use of a compound or pharmaceutical composition of claim 1, or a pharmaceutically acceptable salt or solvate thereof, for the manufacture of a medicament for the treatment or prevention of cancers or tumors.

21. A method of treating or preventing a cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer) colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma) in a subject in need thereof, said method comprising administering to a subject in need of such treatment a therapeutically effective amount of a compound of claim 1, or a pharmaceutically acceptable salt or solvate thereof, or a pharmaceutical composition comprising said compound, salt or solvate thereof.

22. A method for treating and/or preventing a tumor, comprising administering to a patient in need thereof a therapeutically effective amount of the compound or pharmaceutical composition comprising the compound of claim 1, or a pharmaceutically acceptable salt or solvate thereof.
